(19) United States
(12) Patent Application Publication
(10) Pub. No.: US 2026/0263079 A1
(43) Pub. Date: Sep. 10, 2026

(54) SURGICAL CLIP APPLIER INSTRUMENTS WITH ARTICULATING JAWS

(71) Applicant: INTUITIVE SURGICAL OPERATIONS, INC., Sunnyvale, CA (US)

(72) Inventors: Jake A. LUCKMAN, Milford, CT (US); Ronald G. LITKE, Sandy Hook, CT (US); Michael MORROW, Shelton, CT (US); Justin KROM, Southington, CT (US)

(73) Assignee: INTUITIVE SURGICAL OPERATIONS, INC., Sunnyvale, CA (US)

(21) Appl. No.: 19/489,258

(22) PCT Filed: Jun. 3, 2024
(86) PCT No.: PCT/US2024/032221

§ 371 Date: Dec. 1, 2025

Related U.S. Application Data
(60) Provisional application No. 63/505,735, filed on Jun. 2, 2023.
(60) Provisional application No. 63/505,738, filed on Jun. 2, 2023.
(60) Provisional application No. 63/505,740, filed on Jun. 2, 2023.
(60) Provisional application No. 63/505,742, filed on Jun. 2, 2023.
(60) Provisional application No. 63/505,870, filed on Jun. 2, 2023.
(60) Provisional application No. 63/505,875, filed on Jun. 2, 2023.

(51) Int. Cl. A61B 17/122; A61B 17/00

(52) U.S. Cl. CPC A61B 17/122; A61B 17/00234; A61B 2017/00296; A61B 2017/00477

(57) ABSTRACT

A surgical instrument for applying surgical clips to tissue comprises an end effector having first and second jaws that are movable between open and closed positions. The instrument comprises a drive member configured for distal translation from the shaft into the end effector to deliver one or more clips to the first and second jaws. The instrument further comprises at least one actuator rod extending through the shaft lateral of the drive member relative to the longitudinal axis and configured to move the jaws between the open and closed positions. Positioning the jaw actuator lateral of the drive member provides space for the drive member to deliver multiple clips to the jaws without interfering with the opening and closing of the jaws.

### CROSS-REFERENCE TO RELATED APPLICATIONS

This application claims the benefit of the following U.S. Provisional Applications: (1) Ser. No. 63/505,738, filed Jun. 2, 2023; (2) Ser. No. 63/505,740, filed Jun. 2, 2023, (3) Ser. No. 63/505,742, filed Jun. 2, 2023; (4) Ser. No. 63/505,870, filed Jun. 2, 2023; (5) Ser. No. 63/505,875, filed Jun. 2, 2023; and (6) Ser. No. 63/505,735, filed Jun. 2, 2023, the complete disclosures of which are incorporated herein by reference for all purposes.

### BACKGROUND

This description generally relates to endoscopic surgical instruments for dissecting, occluding and/or sealing tissue, and more particularly to endoscopic surgical instruments capable of applying multiple clips to vessels and/or tissue.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools are typically similar to those used in conventional (open) surgery, except that the working end or end effector of each tool is separated from its handle by an extension tube (also known as, e.g., an instrument shaft or a main shaft). The end effector can include, for example, a clamp, grasper, scissor, stapler, cautery tool, linear cutter, or needle holder.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, retroperitoneoscopy, pelviscopy, nephroscopy, cystoscopy, cisternoscopy, sinoscopy, hysteroscopy, urethroscopy, and the like.

Minimally invasive telesurgical robotic systems are being developed to increase a surgeon's dexterity when working on an internal surgical site, as well as to allow a surgeon to operate on a patient from a remote location (outside the sterile field). In a telesurgery system, the surgeon is often provided with an image of the surgical site at a control console. While viewing a three dimensional image of the surgical site on a suitable viewer or display, the surgeon performs the surgical procedures on the patient by manipulating master input or control devices of the control console, which in turn control motion of the servo-mechanically operated slave instruments.

The servomechanism used for telesurgery will often accept input from two master controllers (one for each of the surgeon's hands) and may include two or more robotic arms on each of which a surgical instrument is mounted. Operative communication between master controllers and associated robotic arm and instrument assemblies is typically achieved through a control system. The control system typically includes at least one processor that relays input commands from the master controllers to the associated robotic arm and instrument assemblies and back from the instrument and arm assemblies to the associated master controllers in the case of, for example, force feedback or the like. One example of a robotic surgical system is the DA VINCI™ system commercialized by Intuitive Surgical, Inc. of Sunnyvale, California.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. No. 7,594,912 (filed Sep. 30, 2004), U.S. Pat. No. 6,758,843 (filed Apr. 26, 2002), U.S. Pat. No. 6,246,200 (filed Aug. 3, 1999), and U.S. Pat. No. 5,800,423 (filed Jul. 20, 1995), the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. No. 6,702,805 (filed Nov. 9, 2000), U.S. Pat. No. 6,676,669 (filed Jan. 16, 2002), U.S. Pat. No. 5,855,583 (filed Nov. 22, 1996), U.S. Pat. No. 5,808,665 (filed Sep. 9, 1996), U.S. Pat. No. 5,445,166 (filed Apr. 6, 1994), and U.S. Pat. No. 5,184,601 (filed Aug. 5, 1991), the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

Endoscopic surgical clip appliers are used for a number of minimally invasive or endoscopic surgical procedures to occlude, ligate and/or seal vessels and tissue. Applying surgical clips usually involves compressing the clip over the surgical site, such as a blood vessel. Once applied to the vessel, the compressed surgical clip terminates the flow of fluid therethrough.

Conventional surgical clips are designed to be compressed into a latched or locked position around a grasped vessel or other grasped tissue. Typically, the surgical instrument includes jaws that can be closed to engage bosses formed on the clips. These bosses are forced inwardly about a hinge section causing the first and second legs of the clip being applied to close around the grasped vessel. The tip section of the second leg then begins to contact a hook section. Upon opening of the jaws, the tip section snaps into and is conformably seated in the latching recess, at which point the clip is secured into a latched condition.

Certain endoscopic surgical clip appliers include a surgical instrument having an end effector with movable jaws and a single clip that is installed within the end effector. These instruments are limited to a single discharge per instrument. In other words, once a clip has been discharged and applied to tissue, the surgeon must remove the surgical instrument from the cannula and manually reload a new clip into the instrument, or use a completely different surgical clip applier (i.e., a new instrument).

Other laparoscopic clip appliers have been developed with a cartridge that may be preloaded with about 2-10 clips. These clip appliers, however, are typically disposable and designed to be discarded after a procedure. Some existing endoscopic surgical clip appliers are “straight” or “non-wristed” instruments that do not allow the user to change the orientation of the jaws relative to the shaft of the instrument during, or after, clip advancement.

In addition, the clips in the clip cartridge typically take a “set” in the closed positioned over time (i.e., the legs of the clip tend to move closer towards each other into a closed or semi-closed position while they are stored in the clip cartridge). Unfortunately, these “multi-fire” clip appliers do not have the ability to securely hold the bosses of the clips within the jaws once they are advanced into the jaws. In such event, the clip applier may misfire and drop a clip into the surgical field.

Accordingly, while the new telesurgical systems and devices have proven highly effective and advantageous, still further improvements would be desirable. In general, it would be desirable to provide improved endoscopic clip appliers that are capable of discharging multiple clips without requiring either an instrument exchange or repositioning of the jaws. Additionally, it would be advantageous to provide such improved endoscopic clip appliers without sacrificing the overall instrument size, thereby allowing for the design of compact and maneuverable instruments.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

In one aspect, a surgical instrument for applying surgical clips to tissue comprises an elongate shaft having a longitudinal axis and an end effector coupled to the shaft and including first and second jaws movable between open and closed positions. The instrument comprises a drive member configured for distal translation from the shaft into the end effector to deliver one or more clips to the first and second jaws and an actuator extending through the shaft to move the jaws between the open and closed positions. The actuator is positioned laterally of the drive member relative to the longitudinal axis to provide space for the passage of the drive member and the clips from the shaft to the end effector.

The actuator may comprise a cable drive or similar actuation mechanism that opens and closes the jaws with longitudinal translation of the cable drive (i.e., a push/pull mechanism). In one embodiment, the cable drive comprises an elongate rod having a distal end portion. A slot pin is coupled to the distal end portion. The end effector and/or one of the jaws comprises a slot and the cable drive translates the slot through the slot to move the jaws between the open and closed positions. In certain embodiments, the cable drive may be coupled to first and second slot pins configured to translate through first and second slots in the jaws. Translation of the slot pins through the slots opens and closes the jaws.

In embodiments, the instrument further comprises a wrist assembly rotatably coupling the end effector with the shaft and an internal tube extending through the wrist assembly. At least a portion of the drive member is configured to translate through the internal tube and the cable drive is positioned laterally of the internal tube.

In embodiments, the instrument comprises first and second cable drives extending through the shaft laterally of the drive member. The first and second cable drives may, for example, each be positioned on one side of shaft to accommodate passage of the drive member therethrough. In these embodiments, each of the cable drives are coupled to one or more slot pins in the end effector to open and close the jaws through compressions or tension of the cable drives.

In embodiments, the cable drive(s) comprise a grip cable having a flexible portion that extends through the internal tube in the wrist assembly and first and second substantially rigid portions on either side of the flexible portion. The rigid portions allow for tension and compression of the grip cable to open and close the jaws. The flexible portion is configured to accommodate changes in length and/or angle of the wrist assembly. This allows a surgeon, for example, to deliver one or more surgical clips onto tissue or vessels without having to change the orientation of the jaws during clip advancement, which reduces disruption to the surgeon's workflow. Providing at least one degree of rotational movement relative to the shaft enables the end effector to correspond with at least a portion of the natural action of a surgeon's wrist, thereby facilitating placement of the jaws in the optimal location for performing the sealing/occluding function, particularly in a laparoscopic procedure wherein the instrument has been inserted through a small entry point into the abdominal cavity.

In one such embodiment, the grip cable(s) comprises an outer sheath that is fixed to a first portion of the wrist assembly and is slidingly coupled to a second portion of the wrist assembly. The outer sheath contains the flexible portion to inhibit buckling of the grip cable when the grip cable is under compression (i.e., pushed distally to open or close the jaws).

In certain embodiments, the grip cable(s) may comprise a first layer of flexible tubing between the outer sheath and the grip cable configured to contain the cable strands when under compression. The grip cable(s) may comprise a second layer of flexible tubing overlying the first layer. The second layer of tubing overlaps with the rigid and flexible portions of the grip cable to provide a continuous grip cable outer diameter.

The wrist assembly may, for example, comprise first, second and/or third linkages or discs for rotatably coupling the end effector to the shaft. The outer sheath of the grip cable is preferably secured to the distal linkage or disc and slidingly coupled to the proximal linkage(s) or disc(s).

In one such embodiment, the wrist assembly comprises first and second linkages or discs for articulating the end effector around first and second axes, respectively. The first and second axes may be, for example, yaw and pitch axes. The internal tube extends through the entire wrist assembly, allowing a user to deliver surgical clips onto tissue or vessels through the wrist member so that the end effector can be rotated in at least two axes relative to the shaft, further increasing the ability of the surgeon to reposition the jaws relative to the target vessel or tissue.

In embodiments, the drive member comprises a distal portion configured for removably engaging a clip and a flexible portion that extends through the wrist member when the distal portion is within the end effector. The drive member further comprises a proximal portion extending through the shaft and configured for coupling to an actuator, such as instrument handle or an external control system. The flexible portion allows the distal portion to articulate relative to the proximal portion when the end effector articulates about the wrist member.

In embodiments, the first and second jaws each comprise a guide track extending from the wrist member to a distal end of the jaws. The guide tracks facilitate the advancement of the clip and the engagement elements of the drive member through the jaws and into position such that the jaws can open and/or close the clip.

In another aspect, a surgical instrument for applying surgical clips to tissue comprises an elongate shaft, a wrist assembly and an end effector rotatably coupled to the shaft about the wrist assembly. The end effector includes first and second jaws movable between open and closed positions. The instrument further includes a flexible tube having an internal channel extending through the wrist assembly and a drive member configured to translate longitudinally through the internal channel of the flexible tube to deliver one or more surgical clips to the first and second jaws.

In embodiments, the flexible tube is configured to bend as the wrist assembly articulates relative to the end effector. This provides a flexible, smooth channel for passage of the drive member and the clips therethrough even when the wrist assembly is articulating such that the end effector and the shaft are not oriented in a parallel direction.

In certain embodiments, the wrist assembly is pivotally coupled the end effector about first and second axes, the first and second axes perpendicular to the shaft axis. The flexible tube is configured to bend in at least first and second locations on the flexible tube as the wrist assembly pivots about the first and second axes. In a preferred embodiment, the flexible tube comprises a coil having an elastic polymer sheath or jacket surrounding the coil. The coil provides a structure that inhibits buckling when the wrist assembly pivots and creates tight bends in the flexible tube. The polymer jacket surrounds and bonds to the coil, providing a flexible structure for the coils.

In one embodiment, the flexible tube comprise includes a cross-section with a semi-circular portion and a substantially linear portion that provides a substantially D-shaped cross-section. This cross-section allows the cable drive to extend alongside the linear portion laterally outward from the flexible tube, thereby providing space within the wrist assembly to allow for passage of the drive member and the clips therethrough.

In another embodiment, the flexible tube includes a cross-section with first and second substantially linear portions and first and second semi-circular portions extending between the linear portions. This cross-section allows for first and second cable drives to extend alongside the linear portions of the tube.

In another aspect, a surgical instrument for applying surgical clips to tissue comprises an elongate shaft and an end effector coupled to the shaft and including first and second jaws movable between open and closed positions. The instrument includes a wrist assembly and a drive member configured to translate longitudinal through the wrist assembly to deliver one or more surgical clips to the first and second jaws. The wrist assembly comprises one or more links rotatably coupling the end effector with the shaft about an axis perpendicular to the shaft.

In embodiments, the wrist member comprises a proximal link and a distal link. The distal link may be fixed to the end effector such that the rotation or articulation only occurs between the proximal and distal links of the wrist assembly. This configuration “decouples” the jaws from the wrist assembly, which provides more control and precision for the surgeon in positioning the jaws in a proper orientation for applying a clip to tissue or a vessel.

In embodiments, the wrist assembly further comprises a middle link between the proximal and distal links. The middle link is rotatably coupled to the proximal link about a first axis and rotatably coupled to the distal links about a second axis. This configuration provides both yaw and pitch articulation of the end effector relative to the shaft.

In embodiments, the instrument includes a clip cartridge within the shaft and comprises at least first and second clips spaced from each other generally along the longitudinal axis of the shaft. A drive member is configured to advance the first clip into the end effector, withdraw from the end effector and advance the second clip into the end effector. This allows for multiple clips to be applied to target sites within a patient without exchanging instruments or the clip cartridge.

In embodiments, the drive member comprises at least one engagement element for removably coupling to the first and second clips. In one embodiment, the drive member is disposed within the clip cartridge, which, for example, may be disposable. In another embodiment, the drive member is movably coupled to the shaft, which, for example, may be reusable.

In embodiments, the jaws each include an engagement feature at their distal ends to secure the clips therein after they have been delivered by the drive member. The engagement features allow the drive member to be released from the clip after the clip has been secured to jaws. Thus, the force required to disengage the drive member from the clip is less than the force required to disengage the clip from the engagement features. In addition, these engagement features ensure that the clip does not fall out of the jaws before they have been closed and latched onto tissue or a vessel at the target site. In a preferred embodiment, these engagement features comprise ramped leaf springs configured to secure each of the two arms of the clip to the first and second jaws, respectively.

In embodiments, the drive member is configured to remain coupled to the surgical clip when the first and second jaws move between the open and closed positions. The drive member retains and controls the clip, which allows the drive member to position the clip arms within the jaws of the end effector and to retain the clip while the end effector is opened and closed and/or articulated relative to the shaft of the instrument. This allows the surgeon to fully open the clips after they have been advanced into the jaws so that they can be effectively positioned around a target vessel or tissue. In addition, this allows the surgeon to reposition the jaws relative to the shaft after the clip has been advanced into the jaws.

In one embodiment, the engagement elements of the drive member are configured to removably couple to each of the two arms of the clip to provide additional control of the clip within the jaws of the instrument. The clip comprises first and second arms pivotally coupled to each other about a hinge and movable between open and closed positions relative to each other. The clip further comprises first and second engagement members on the first and second arms, respectively. These engagement members are configured for removable coupling to the first and second engagement elements of the drive member. The engagement elements are preferably located near, or at, the distal end portions of each of the arms (or the ends opposite the hinge).

In another embodiment, the engagement elements of the drive member are configured to removably couple to a proximal portion of the clip. The engagement elements may, for example, comprise first and second tabs that are biased inwardly to secure to the hinge portion of the clip.

In embodiments, the first and second jaws each comprise a guide track. The first and second engagement elements are configured to advance along the guide tracks as the drive member is advanced distally into the jaws. This ensures that the first and second arms of the clip are optimally positioned on either jaw even if the jaws are open, and/or if the jaws are articulated relative to the shaft as the clip is advanced thereto.

In certain embodiments, the drive member is coupled to an actuator configured to translate the drive member in the proximal and distal directions. The actuator may, for example, include a handle of the surgical instrument that allows the surgeon to manually advance and withdraw the drive member and/or open and close the jaws of the instrument.

In embodiments, the actuator is configured for coupling to a robotic teleoperated control system. The robotic teleoperated control system may comprise a control system coupled to the actuator and configured to translate the drive member proximally and distally relative to the end effector. In addition, the control system may include one or more actuators for opening and closing the jaws. For example, in one configuration, the actuator will be manipulated by the robotic manipulator assembly to move the jaws of the end effector between an open position and a closed position. In the closed position, the jaws are actuated into compressing contact with the legs of a clip, thereby compressing the clip into a latched or locked position around a vessel or other tissue.

In embodiments, the control system may monitor and control the longitudinal location of the drive member relative to each of the clips within the cartridge. In particular, the control system may monitor the location of engagement elements along cartridge to determine when the drive member should be translated distally or proximally.

It is to be understood that both the foregoing general description and the following detailed description are exemplary and explanatory only and are not restrictive of the description. Additional features will be set forth in part in the description which follows or may be learned by practice of the description.

### BRIEF DESCRIPTION OF THE DRAWINGS

The above and other aspects, features, and advantages of the present surgical instruments will become more apparent in light of the following detailed description when taken in conjunction with the accompanying drawings in which:

FIG. 1 is a perspective view of a distal portion of a surgical instrument;

FIG. 2 is an exploded view of the surgical instrument of FIG. 1 with a clip cartridge configured for insertion through a longitudinal slot in the side of instrument shaft;

FIGS. 3A and 3B illustrates a surgical instrument with a clip cartridge configured for insertion through a proximal end of the instrument;

FIG. 4 is a partial cross-sectional view of the surgical instrument of FIG. 1;

FIG. 5 illustrates an enlarged view of a portion of a clip cartridge and a drive member of the surgical instrument;

FIG. 6A illustrates the drive member of the surgical instrument;

FIG. 6B illustrates distal engagement elements of the drive member;

FIGS. 7A-7E illustrate alternative embodiments of drive members for the surgical instrument;

FIG. 8A illustrates a clip in the open position;

FIG. 8B illustrates a clip in the closed position;

FIG. 8C is a top view of one arm of a clip;

FIG. 8D is a side view of the clip in a partially closed position;

FIG. 9A is a side view of a distal hook on one arm of the clip;

FIG. 9B is a front view of a distal latch on another arm of the clip;

FIG. 9C is a front view of the distal hook;

FIG. 9D is a back view of the distal latch;

FIG. 9E is a close-up view of the hook and the latch in a closed position;

FIG. 10A illustrates first and second jaws of the instrument of FIG. 1, with a clip attached to the drive member within the jaws;

FIG. 10B illustrates the jaws in an open position;

FIG. 10C illustrates the jaws in a closed position;

FIG. 10D is a top view of one of the jaws illustrating the four-bar linkage of the jaws;

FIG. 10E is a cross-sectional view of one of the jaws illustrating ramped leaf springs at the distal ends of the jaw;

FIG. 11A is a cross-sectional view of the first and second jaws articulated in a yaw direction relative to the shaft;

FIG. 11B is a cross-sectional view of the end effector of the instrument, illustrating the drive member and the clip being advanced through a wrist assembly and into the jaws of the instrument;

FIG. 12A is a cross-sectional view of a portion of the surgical instrument, illustrating the clip cartridge and the drive member;

FIG. 12B is a close-up view of clip cartridge and the drive member, illustrating the distal engagement elements of the drive member in a distal position relative to upper and lower ramps on the clip cartridge;

FIGS. 13A and 13B illustrate the distal engagement elements of the drive member after proximal retraction to a proximal position relative to a distal set of upper and lower ramps on the clip cartridge;

FIGS. 14A and 14B illustrate the distal engagement elements of the drive member passing through openings in the outer surface of the clip cartridge;

FIGS. 15A-15C illustrate the distal engagement elements of the drive member engaging with engagement features on the first or distal-most clip in the clip cartridge;

FIGS. 16A and 16B illustrate the drive member advancing the first clip through a wrist of the surgical instrument and into the jaws;

FIG. 17 illustrates the clip and the drive member engaged to the first and second jaws, with the jaws in the open position;

FIG. 18 illustrates the first and second jaws in an articulated position about the wrist relative to the shaft;

FIG. 19A illustrates the clip and the drive member engaged to the first and second jaws, with the jaws in the closed position;

FIG. 19B is a close-up view of the latch of the clip and the drive member engaged to one of the jaws;

FIG. 20A illustrates the drive member being retracted from the end effector of the instrument after the clip has been latched;

FIG. 20B is a close-up view of the clips and the jaws in the closed position after the clips has been latched;

FIG. 21 illustrates the drive member positioned for proximal retraction to engage a second clip in the clip cartridge;

FIG. 22 is a perspective view of a distal portion of an alternative embodiment of a surgical clip applier instrument;

FIG. 23 illustrates the surgical instrument of FIG. 22 with a clip cartridge configured for insertion through a longitudinal slot in the side of instrument shaft;

FIG. 24 illustrates the surgical instrument of FIG. 22 with a clip cartridge configured for insertion through a proximal end of the instrument;

FIG. 25 is an exploded view of the clip cartridge and the surgical instrument of FIG. 22;

FIG. 26A is a cross-sectional view of the surgical instrument of FIG. 22 with the clip cartridge installed therein;

FIG. 26B is an enlarged view of a portion of the surgical instrument, illustrating the clip cartridge and a drive member, as well as the distal engagement elements of the drive member engaging with engagement features on the first or distal-most clip in the clip cartridge;

FIG. 26C is an enlarged view of the proximal engagement elements of the drive member and an individual clip within a cartridge;

FIG. 27 illustrates a clip cartridge configured for loading into a longitudinal slot or a proximal opening of the surgical instrument of FIG. 22;

FIG. 28 illustrates a clip cartridge configured for loading into a proximal opening of the shaft of the surgical instrument of FIG. 22;

FIGS. 29A-29C are enlarged views of the distal portion of the clip cartridge and an individual clip;

FIG. 30 illustrates the drive member of the surgical instrument;

FIG. 31 illustrates a distal portion of the drive member of FIG. 30;

FIG. 32 illustrates a proximal portion of the drive member of FIG. 30;

FIGS. 33A-33C illustrate alternative embodiments of drive members for the surgical instrument;

FIG. 34 illustrates a clip in a partially open position;

FIG. 35A illustrates the clip in a fully open position;

FIG. 35B illustrates a clip in the closed position;

FIGS. 36A and 36B illustrate the drive member coupled to a proximal portion of the clip;

FIG. 37A illustrates another embodiment of a clip;

FIG. 37B illustrates another embodiment of a drive member for use with the clip of FIG. 37A;

FIGS. 38A-38C illustrate another embodiment of a clip and a drive member;

FIGS. 39A and 39B illustrate yet another embodiment of a clip and a drive member;

FIG. 40A illustrates first and second jaws of a surgical clip applier instrument in an open position;

FIG; 40B illustrates the first and second jaws in a closed position;

FIG. 40C is a partial cross-sectional view of the jaws in the open position;

FIG. 40D is a partial cross-sectional view of the jaws in the closed position;

FIG. 41A illustrates another embodiment of the first and second jaws in an open position;

FIG. 41B illustrates the jaws of FIG. 41A in the closed position;

FIG. 42 illustrates a wrist assembly and a drive cable for a surgical clip applier;

FIG. 43A is a partial cross-sectional view of the wrist assembly and drive cable of FIG. 42;

FIG. 43B is a partial cross-sectional view of the wrist assembly and drive cable with the wrist assembly articulated relative to the shaft of the instrument;

FIG. 44A illustrates an internal tube within the wrist assembly;

FIG. 44B illustrates an alternative embodiment of an internal tube for the wrist assembly;

FIG. 45A is a perspective view of the internal tube of FIG. 44A;

FIG. 45B illustrates the internal tube within the wrist assembly during articulation of the wrist assembly;

FIG. 46 is a perspective view of a drive cable for opening and closing the jaws;

FIG. 47A illustrates the drive cable with parts removed;

FIG. 47B illustrates the drive cable with a heat shrink tubing over a flexible portion of the drive cable;

FIG. 47C illustrates the drive cable with another layer of heat shrink tubing;

FIG. 48A is a cross-sectional view of the instrument, illustrating the drive member advancing a clip through the shaft of the instrument;

FIG. 48B is an enlarged view of the drive member coupled to the clip;

FIG. 49A is a cross-sectional view of the instrument; illustrating the drive member advancing the clip through the wrist assembly;

FIG. 49B is an enlarged view of FIG. 49A;

FIG. 49C illustrates the drive member advancing the clip through the wrist assembly as the wrist is articulated relative to the shaft;

FIG. 49D is a partial cross-sectional view of FIG. 49C;

FIGS. 50A and 50B illustrate the clip advancing through guide tracks in the jaws;

FIG. 51 illustrates the clip secured to the distal ends of the jaws as the drive member is withdrawn proximally from the jaws;

FIGS. 52A-52C illustrate the jaws in the closed position, latching the clip;

FIG. 53 is a perspective view of the internal tube in an articulated orientation;

FIGS. 54A-54C illustrate a clip advancing through the wrist assembly in a substantially orthogonal orientation relative to the wrist axis;

FIGS. 55A-55C illustrate a clip advancing through the wrist assembly in a substantially 45 degree orientation relative to the wrist axis;

FIG. 56 is a perspective view of a representative teleoperated surgical instrument usable with an exemplary embodiment of the present teachings;

FIG. 57 illustrates a top view of an operating room employing a robotic surgical system;

FIG. 58 illustrates a simplified side view of a robotic arm assembly; and

FIG. 59 is a flow chart of a process for operating a surgical clip applier instrument with a control system.

### DETAILED DESCRIPTION

Particular embodiments of the present surgical instruments are described hereinbelow with reference to the accompanying drawings; however, it is to be understood that the disclosed embodiments are merely exemplary and may be embodied in various forms. Therefore, specific structural and functional details disclosed herein are not to be interpreted as limiting, but merely as a basis for the claims and as a representative basis for teaching one skilled in the art to variously employ the devices herein in virtually any appropriately detailed structure. Well-known functions or constructions are not described in detail to avoid obscuring the present description in any unnecessary detail. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment Moreover, the depictions herein are for illustrative purposes only and do not necessarily reflect the actual shape, size, or dimensions of the system or illustrated components.

While the following is presented with respect to surgical instruments that are compatible with surgical clip cartridges, it should be understood that certain features of the presently described surgical instruments may be readily adapted for use in any type of surgical clamping, cutting, ligating, dissecting, clipping, cauterizing, suturing and/or sealing instrument, whether or not the surgical instrument applies a clip or other type of fastener. Additionally, the features of the presently described surgical ligating instruments may be readily adapted for use in surgical instruments that are actuated using any technique within the purview of those skilled in the art, such as, for example, manually activated surgical instruments, powered surgical instruments (e.g., electro-mechanically powered instruments), robotic surgical instruments, and the like.

The devices described herein, or certain components of the devices, may also be incorporated into a variety of different surgical instruments, such as those described in commonly assigned, co-pending US. Patent Application Nos. Ser. No. 16/205,128, Ser. No. 16/427,427, Ser. No. 16/678,405, Ser. No. 16/904,482, Ser. No. 17/081,088 and Ser. No. 17/084,981 and International Patent Nos. PCT/US2019/107646, PCT/US2019/019501, PCT/US2019/062344, PCT/US2020/54568, PCT/US2019/064861, PCT/US2019/062768, PCT/2020/025655, PCT/US2020/056979, PCT/2019/066513, PCT/US2020/020672, PCT/US2019/066530 and PCT/US2020/033481, the complete disclosures of which are incorporated by reference herein in their entirety for all purposes as if copied and pasted herein.

FIG. 1 illustrates the distal end portion of a surgical instrument 100 in accordance with an illustrative embodiment. Surgical instrument 100 includes an end effector 110, an elongated shaft 105 and a wrist assembly 140 coupling end effector 110 to shaft 105. The proximal end portion of elongate shaft 105 is operatively connected to an actuation mechanism (not shown), although as those skilled in the art reading this description will appreciate, components of the actuation mechanism may extend into, and/or pass through elongated shaft 105 and/or wrist assembly 140.

In certain embodiments, the surgical instruments described herein are adapted to be used with a robotic system for applying ligating clips. The surgical instruments will generally include an actuation mechanism that controls the orientation and movement of the end effector. The actuation mechanism will typically be controlled by a robotic manipulator assembly that is controlled remotely by a user. For example, in one configuration, the actuation mechanism will be manipulated by the robotic manipulator assembly to move the jaws of the end effector between an open position and a closed position. In the closed position, the jaws are actuated into compressing contact with the legs of a clip, thereby compressing the clip into a latched or locked position around a vessel or other tissue

End effector 110 includes a first jaw 111 and a second jaw 112 configured to move between an open position (as shown in FIG. 1) where the jaws are spaced apart from one another and a closed position to force the jaws into compressing contact with the legs of a clip to close and seal the clip around vessels or tissue. In certain embodiments, second jaw 112 is a movable jaw configured to move from an open position to a closed position relative to first jaw 111. In other embodiments, first jaw 111 is a movable jaw configured to move between open and closed positions relative to second jaw 112. In still other embodiments, both jaws 111, 112 are movable relative to each other.

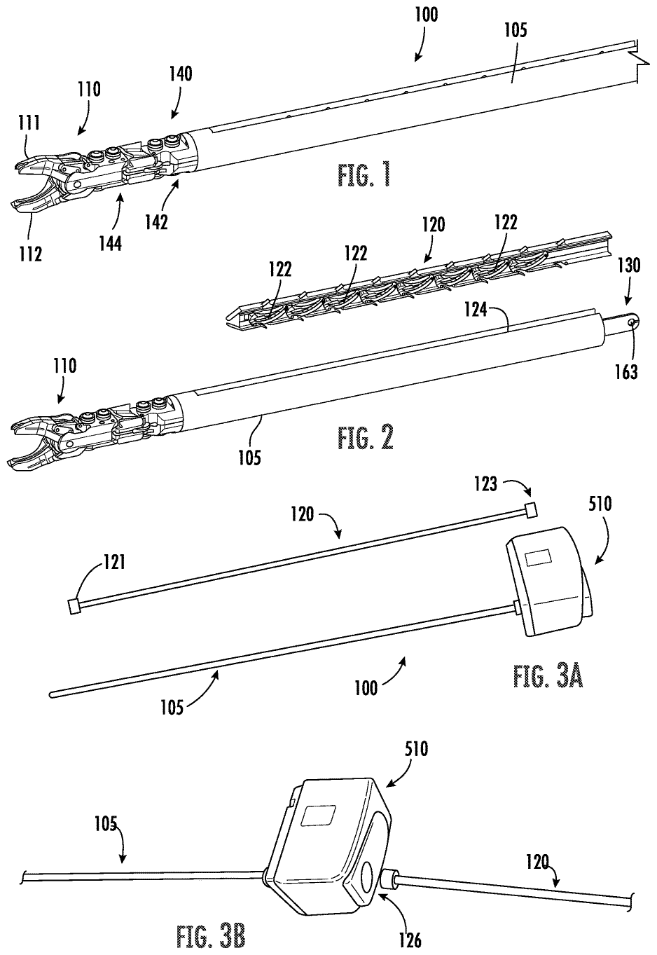

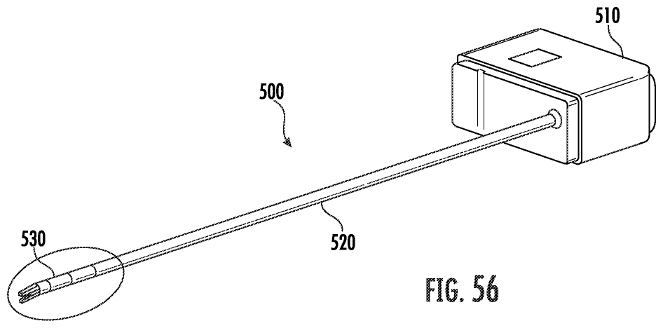

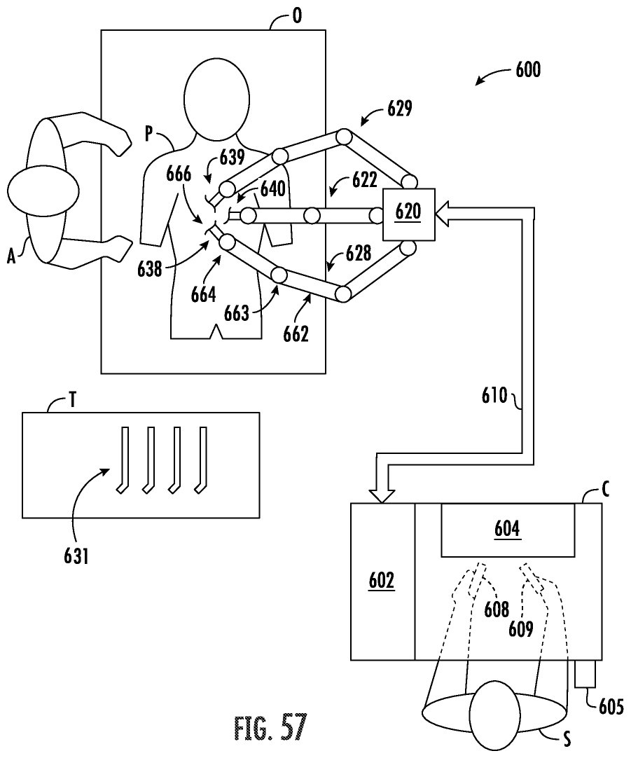

The actuation mechanism may include input couplers (not shown) instead of, or in addition to, the stationary and movable handles. In certain embodiments, surgical instrument 100 will further include a backend mechanism 510 (see FIGS. 3A and 56) coupled to the proximal end portion of elongate shaft 105. The backend mechanism typically provides a mechanical coupling between the drive tendons, rods or cables of the instrument and motorized axes of the mechanical interface of a drive system. Further details of known backend mechanisms and surgical systems are described, for example, in U.S. Pat. Nos. 8,597,280, 7,048,745, and 10,016,244. Each of these patents is hereby incorporated by reference in its entirety.

The input couplers may interface with, and be driven by, corresponding output couplers (not shown) of a telesurgical surgery system, such as the system disclosed in U.S Pub. No. 2014/0183244A1, the entire disclosure of which is incorporated by reference herein. The input couplers are drivingly coupled with one or more input members (not shown) that are disposed within the instrument shaft 105. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. Nos. 8,597,280, 7,048,745, and 10,016,244. Each of these patents is hereby incorporated by reference in its entirety for all purposes.

While described herein with respect to an instrument configured for use with a robotic surgical system, it should be understood that the actuation and drive assemblies described herein may be incorporated into manually actuated instruments, electro-mechanical powered instruments, or instruments actuated in any other way. For example, the actuation mechanism may comprise a handle assembly for gripping by the user that includes a stationary handle and a moveable handle, which serves as an actuator for surgical instrument 100.

Referring now to FIGS. 2, 3A and 3B, instrument 100 may be provided with a clip cartridge 120 that comprises a plurality of surgical clips 122 and is installed into surgical instrument 100. In certain embodiments, cartridge 120 may be installed through a longitudinal slot 124 in the side of shaft 105 (FIG. 2). In other embodiments, cartridge 120 may be installed through a proximal opening 126 of a manual handle or a backend mechanism 510 of the instrument (see FIGS. 3A and 3B). In this latter embodiment, the staple cartridge 120 includes an enlarged distal end 121 that facilitates advancement of cartridge 120 through an internal channel (not shown) in backend mechanism 510 and an internal lumen (not shown) of instrument shaft 105. Staple cartridge 120 may also include a proximal handle 123 that facilitate the user's grip on cartridge 120, allowing the user to advance distal end 121 through the shaft 105 to a suitable position just proximal of end effector 110.

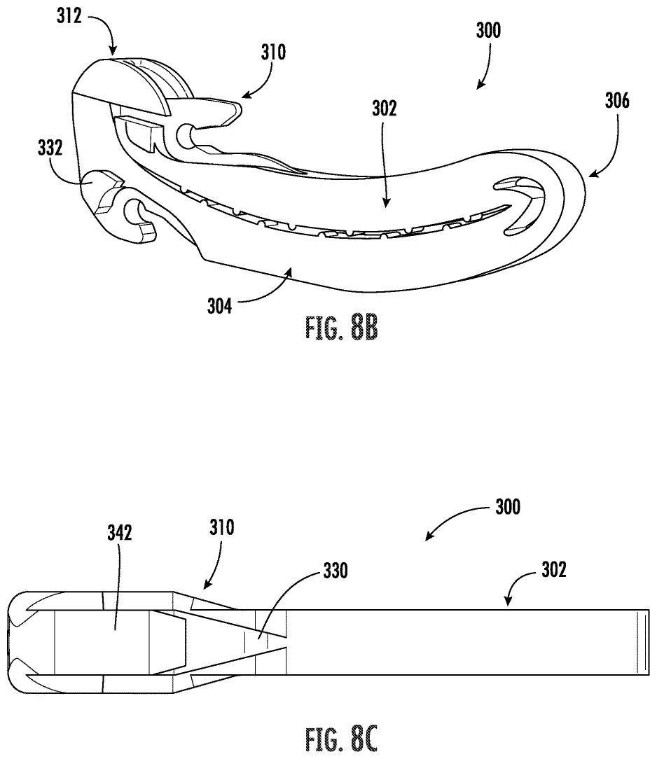

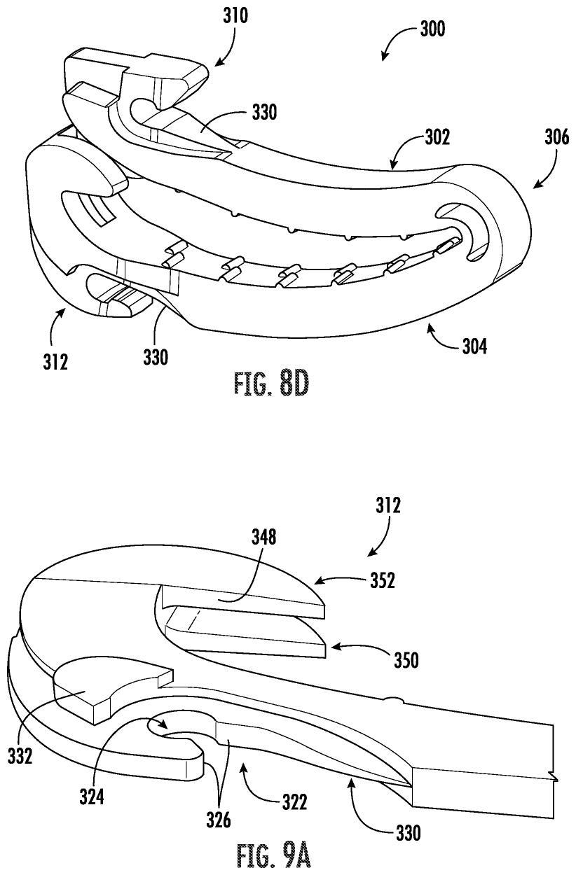

Cartridge 120 may contain between about 1 to 20 clips, preferably between about 2 to 12 clips. Clip 122 preferably extend in a substantially parallel direction relative to the longitudinal axis of shaft 105. Cartridge 120 may be constructed from any suitable materials known in the art, such as a single-molded plastic body or a sheet metal construction. Cartridge 120 may be adapted to accommodate any suitable desired sizes and configurations of clips 122, including conventional clips (e.g., titanium, tantalum, or stainless steel ligation clips, such as the Horizon™, Hemoclip® or the like and/or polymer clips, such as the Vas-Q-Clip®, the Weck@ Hem-o-lok® or the like). Alternatively, cartridge 120 may be adapted to accommodate the novel clips 300 described below and shown in FIGS. 8A-9D and FIGS. 34-39B.

Wrist assembly 140 is positioned between end effector 110 and elongated shaft 105. Wrist assembly 140 may provide a desired amount of motion, such as +/−90 degrees in a pitch, yaw and/roll direction, preferably +/−about 60 to about 65 degrees in the pitch and yaw directions. Cables or other actuators (not shown) are drivingly coupled with the wrist assembly 140 and actuated to impart motion to wrist assembly 140. Differential movement of the cables can be used to actuate wrist assembly 140 to pitch and yaw at various angles. Additional details of articulation mechanisms usable with the embodiments disclosed herein are disclosed in Int'l. Pub. No. WO 2015/127250A1 and U.S. Publication No. 2017/0215977 A1, the complete disclosure of which is incorporated herein by reference for all purposes.

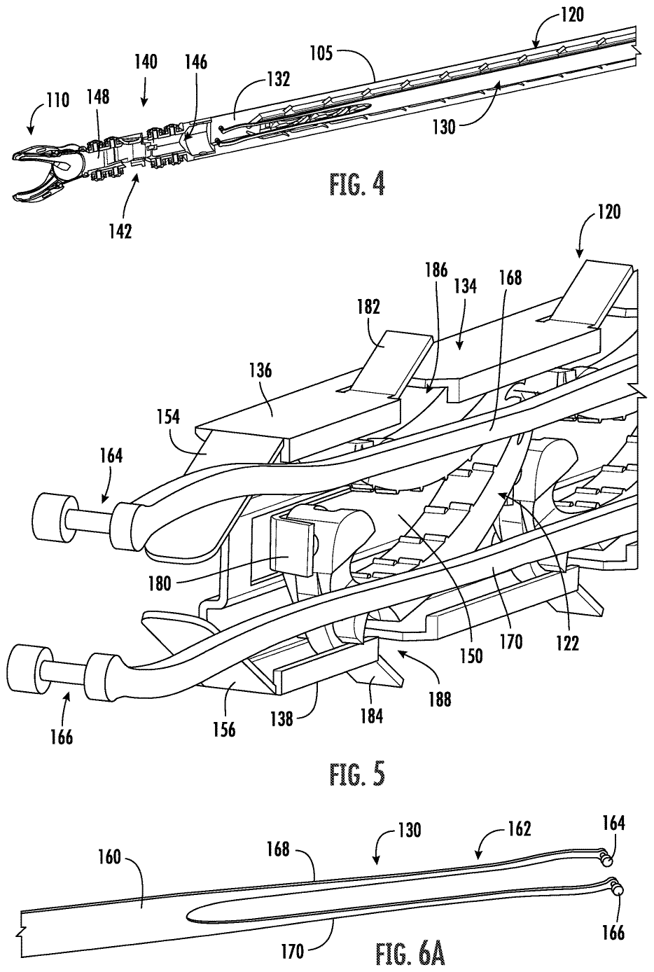

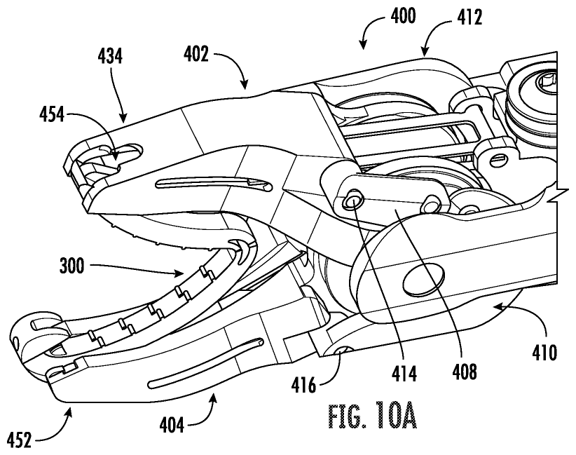

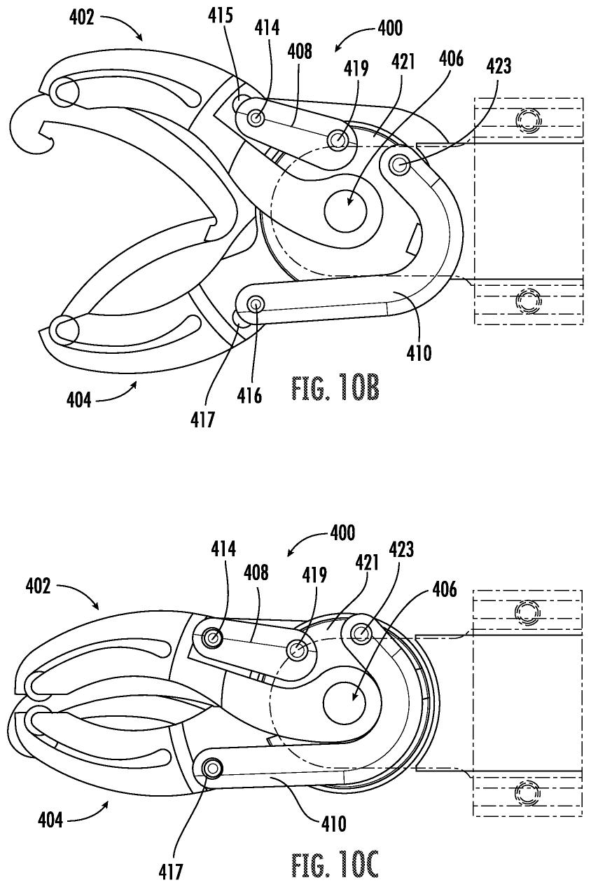

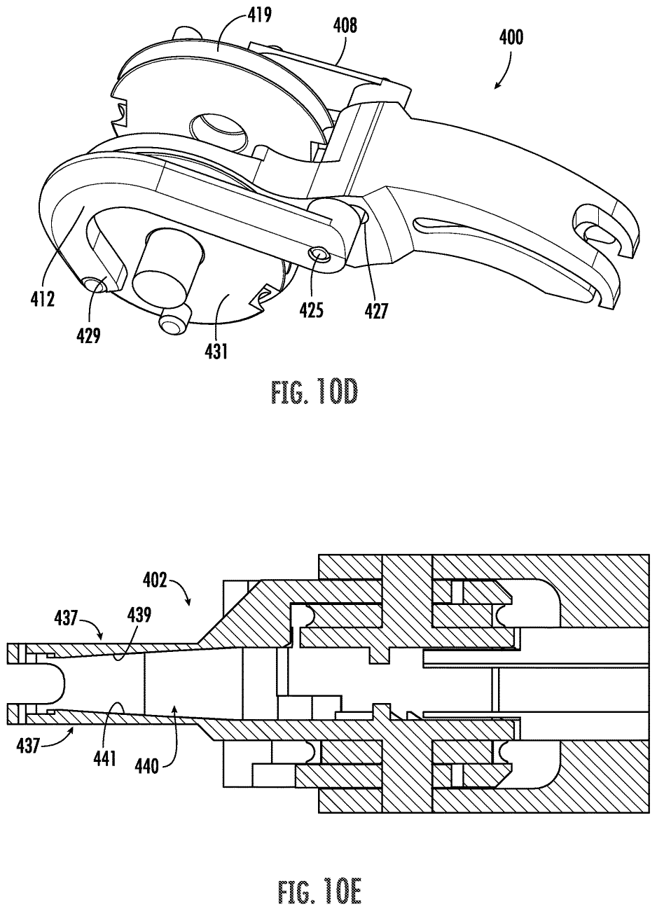

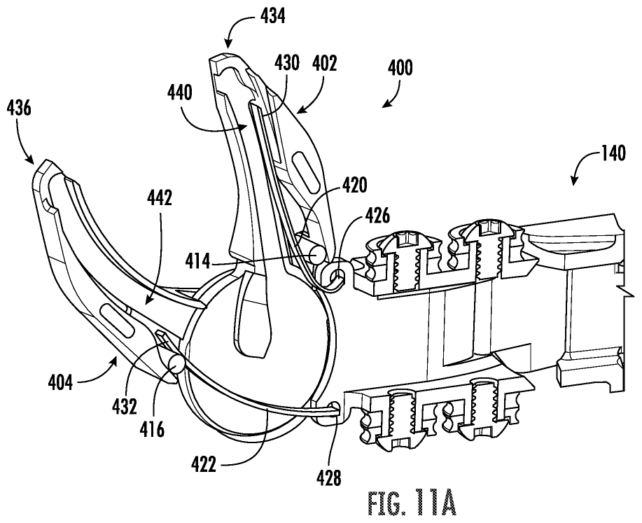

In one embodiment, wrist assembly 140 may include a linkage 142 that provides the pitch motions of the wrist assembly 140. For yaw motions of wrist assembly 140, the pulleys 419, 431 and linkages 408, 412 (discussed below in reference to FIG. 10D) are rotated together about a single axis. As shown in FIGS. 4 and 16A, wrist assembly 140 has first and second internal lumens 146, 148 to allow for movement of a drive member 130 and clips 122 therethrough.

Referring now to FIG. 4, cartridge 120 is configured to extend through an internal lumen 132 in shaft 105, preferably along one side of shaft 105 (i.e., substantially on one side of the longitudinal axis of shaft 105). Instrument 100 further comprises a drive member (or clip advancer) 130 that preferably extends through lumen 132 adjacent to cartridge 120. In certain embodiments, drive member 130 is disposed on the opposite side of the longitudinal axis from cartridge 120, which allows drive member 130 to translate proximally and distally within shaft 105 relative to cartridge 120, as discussed below. In other embodiments, drive member 130 and cartridge 120 may be disposed on the same side of the longitudinal axis, or cartridge 120 may be centered within lumen 132 along the longitudinal axis and drive member 130 disposed adjacent to cartridge 120 on either side.

In certain embodiments, drive member 130 is coupled to instrument shaft 105 such that drive member 130 is included as part of the overall instrument 100, which may be constructed of materials designed for re-use of the instrument in multiple surgical procedures. In other embodiments, drive member 130 is coupled to cartridge 120 such that drive member 130 is included as part of the clip cartridge 120, which may be constructed of materials designed for disposable or single-use applications. In either embodiment, drive member 130 is configured for longitudinal displacement relative to shaft 105 to advance clips 122 from cartridge 120 to jaws 111, 112 of end effector 110, as discussed in more detail below.

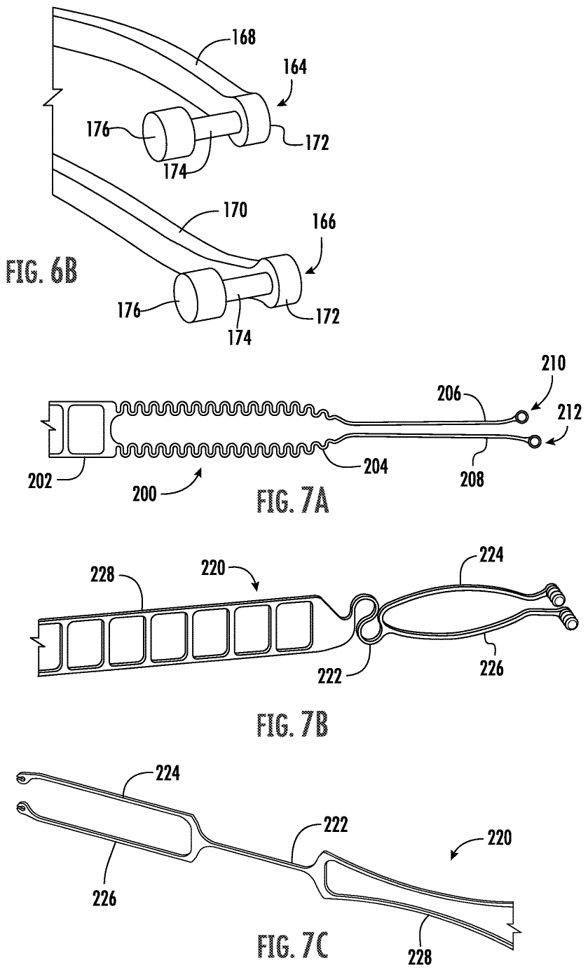

As shown in FIGS. 6A and 6B, drive member 130 comprises a proximal component 160 and a flexible component 162 coupling proximal component 160 with first and second clip engagement elements 164, 166. Proximal component 160 is configured to extend through shaft 105, and may have one or more proximal interfaces 163 (see FIG. 2) for cooperating with an actuation mechanism (not shown) to advance drive member 130 distally and proximally relative to shaft 105. In certain embodiments, a distal portion of proximal component 160 may extend through a portion of wrist assembly 140. For example, in one such embodiment, this distal portion of proximal component 160 extends far enough through wrist assembly 140 to flex in at least the pitch direction, but typically not in the yaw direction. Flexible component 162 will typically flex in both the pitch and yaw directions.

Flexible component 162 preferably comprises a material that is stiff enough to push through wrist assembly 140 into jaws 111, 112. At the same time, these components comprise a material that is flexible and resilient enough to bend as end effector 110 is articulated relative to shaft 105 at wrist assembly 140. In a preferred embodiment, these components comprise nitinol, polymers, such as PEEK, spring steel or similar materials.

In one embodiment, flexible portion 160 comprises first and second arms 168, 170 that each include a clip engagement element 164, 166 at the distal ends thereof. Engagement elements 164, 166 are configured to extend laterally away from arms 168, 170 such that they are positioned substantially parallel with clip cartridge 120 within shaft 105 (see FIG. 5) for engaging with clips 122 (discussed below). Arms 168, 170 are preferably designed to move relative to each other between a first position, wherein the arms are closer to each other (see FIG. 6A), and a second position, wherein the arms are further apart from each other relative to the longitudinal axis of shaft 105 (see, for example, FIG. 12B). In certain embodiments, the arms 168, 170 are substantially parallel to each other in the first position. This allows arms 168, 170 to move into various positions relative to clip cartridge 122 that allow engagement elements 164, 166 to engage one or more clips 122 housed within cartridge 120.

In one embodiment, engagement elements 164, 166 each comprise a first disc portion 172 that is coupled to, or integral with, arms 168, 170. Elements 164, 166 further comprise a central shaft 174 extending laterally away from disc portion 172 and coupled to a second disc portion 176 (thereby forming a shape substantially resembling a “dumbbell”). The first and second disc portions 172, 176 of engagement elements 164, 166 preferably have a larger diameter than central shaft 174, which enables shaft 174 to removably couple to clips 122, as discussing in more detail below.

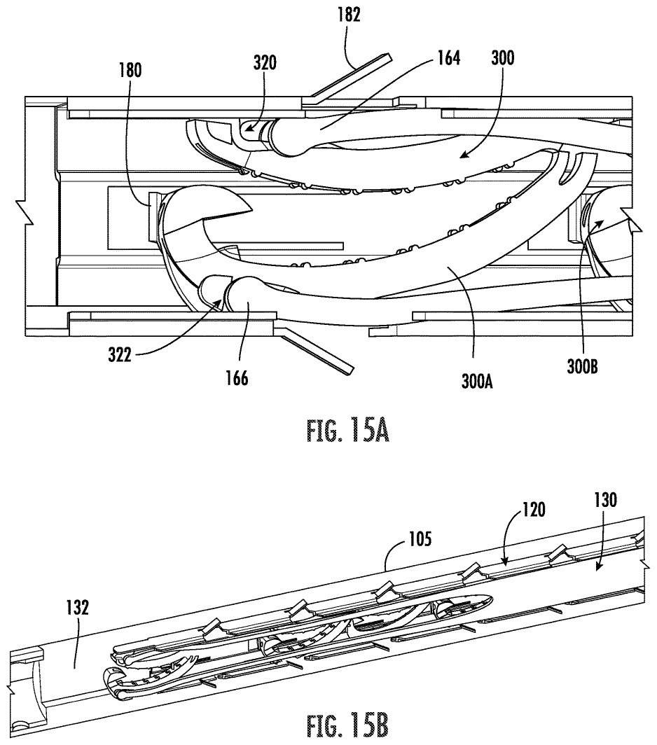

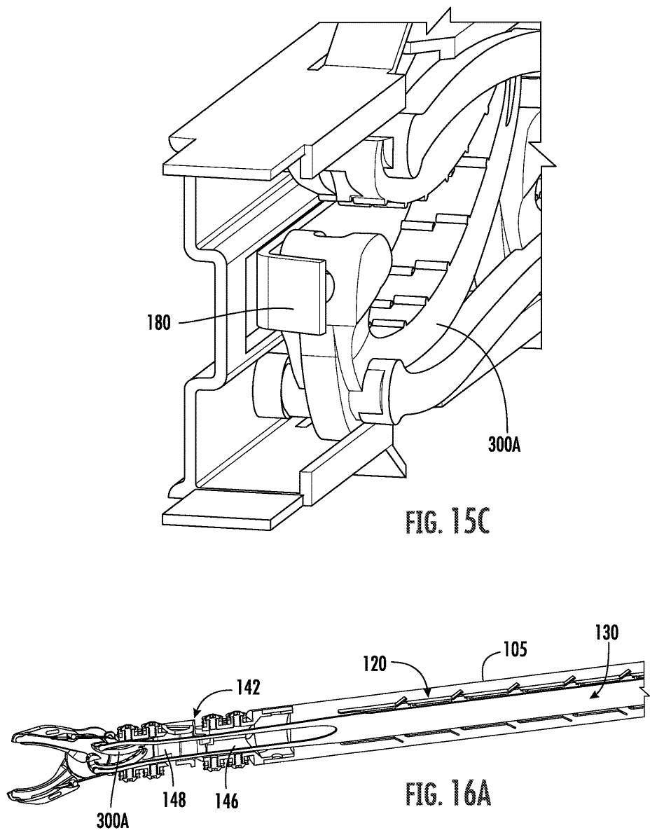

As shown in FIG. 5, clip cartridge 120 comprises a housing 134 having upper and lower walls 136, 138 and a longitudinal wall 150 on one side of housing 134 opposite drive member 130. The side of housing 134 opposite wall 150 is preferably open such that clips 122 can be viewed from this side of housing 134 and to allow drive member 130 to interact with the clips within cartridge 130. Housing 134 further includes first and second distal ramps or tabs 154, 156 extending from the distal end of housing 134. Tabs 154, 156 have sufficient rigidity to force engagement elements 164, 166 of drive member 130 to spread out and ride upwards along tabs 154, 156 when drive member 130 is pulled in the proximal direction (discussed below). At the same time, tabs 154, 156 have sufficiently flexibility to allow engagement elements 164, 166 to push through tabs 154, 156 and advance clips 122 through the distal end of cartridge 120 when drive member 130 advances in the distal direction and after drive member 130 is located within the interior of housing 134 (see FIG. 15B).

Housing 134 further includes retainer tabs 180 extending from longitudinal wall 150 into the interior of cartridge 120. Retainer tabs 180 are spaced from each other longitudinally along housing 120 so as to define discrete areas for retaining each clip 122 within housing 134 (see FIG. 12A). Retainer tabs 180 preferably have sufficient rigidity to hold clips 122 in place within cartridge 120, while having sufficient flexibility such that distal translation of drive member 130 causes clips 122 to bend tabs 180 and allow each clip 122 to separately advance with drive member 130 in the distal direction.

Housing 134 includes a series of upper and lower ramps or tabs 182, 184 that extend away from upper and lower walls 136, 138, respectively, in the proximal direction. Similar to internal tabs 180, upper and lower tabs 182, 184 are spaced from each other longitudinally along housing 120 such that they are disposed above and below each clip 122 within housing 134. In certain embodiments, tabs 182, 184 are pivotally coupled to upper and lower walls 136, 138 to allow for proximal movement of engagement elements 164, 166 over tabs 182, 184. In other embodiments, tabs 182, 184 are substantially stationary ramps. In these embodiments, arms 168, 170 of drive member 130 are configured to separate further away from each other such that elements 164, 166 ride along ramps 182, 184 as drive member 130 is translated in the proximal direction.

Housing 134 further includes upper and lower openings 186, 188 in upper and lower walls 136, 138 located proximally of each upper and lower tab 182, 184. These tabs 182, 184 and openings 186, 188 allow engagement elements 164, 166 to withdraw proximally over tabs 182, 184 and move into the interior of cartridge housing 134 through openings 186, 188, as discussed in more detail below. In addition, each set of tabs and openings provides a discrete location on the cartridge housing associated with one of the clips. In certain embodiments, the instrument or system may include a control system that detects when the engagement elements 164, 166 of drive member 130 are located adjacent to each of the clips within cartridge. This ensures that the user engages the distal-most clip within cartridge.

In an alternative embodiment, each of the clips within the cartridge may be advanced distally simultaneously with each other. For example, the clips may be spaced substantially equally from each other and the distal-most clip may be spaced from the jaws a distance substantially equal to the spacing between the clips. This allows the drive member 130 (or another drive member, such as a shuttle component (e.g., a ratchet and a pawl) or a spring (such as a magazine spring) to move all of the clips forward distally the same distance, thereby allowing, for example, the drive member to advance the distal-most clip to jaws 111, 112 while the next clip is moved to the location previously possessed by the distal-most clip, etc. Thus, the drive member can be withdrawn proximally to the same longitudinal position within the instrument in order to couple with each clip within the cartridge, thereby increasing the speed and efficiency of delivering multiple clips to a target site.

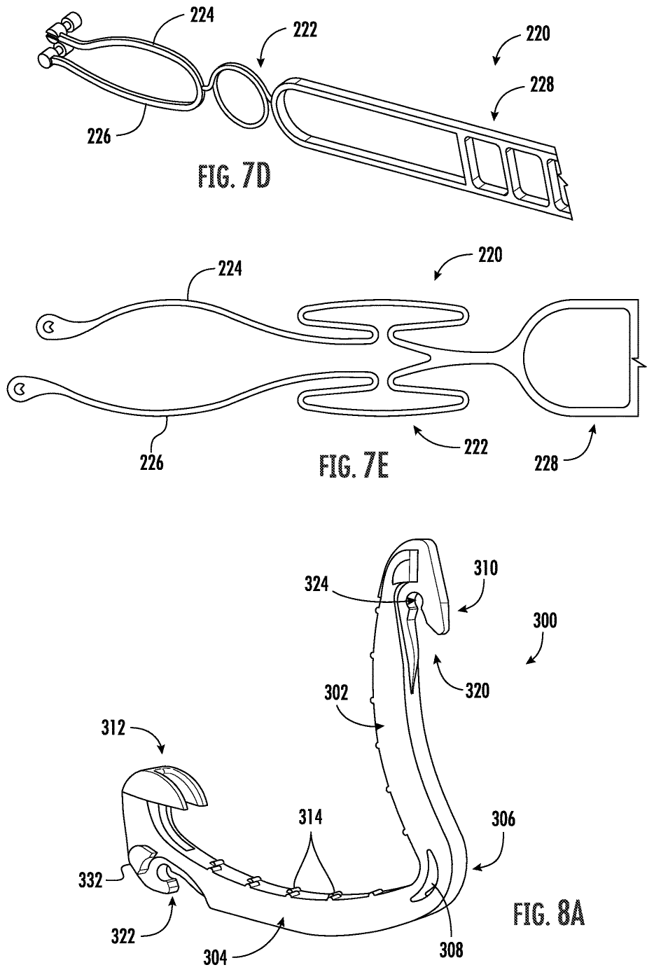

FIGS. 7A-7E illustrate alternative embodiments of drive member 130. As shown in FIG. 7A, a drive member 200 comprises a proximal component 202, a flexible component 204 and first and second arms 206, 208 having distal engagement elements 210, 212. In this embodiment, flexible component 204 comprises wave or coiled features that allow arms 206, 208 to deflect towards and away from the longitudinal axis without yielding or permanent deformation of the material.

FIGS. 7B-7E illustrate alternative embodiments of drive members 220 that includes flexible portions 222 designed to provide a more defined pivot point for each drive member 210. The pivot point of flexible portions 222 is configured to be located within wrist assembly 140 of instrument 100 such that the distal arms 224, 226 are capable of pivoting relative to the proximal component 228 of each drive member 210 when end effector 110 is articulated relative to shaft 105. In some embodiments, arms 224, 226 may be configured to naturally extend substantially parallel to each other (FIG. 7C). In other embodiments, arms 224, 226 may be configured to naturally have a bowed configuration that facilitates the opening and closing of the arms 224, 226 (FIGS. 7B, 7D and 7E).

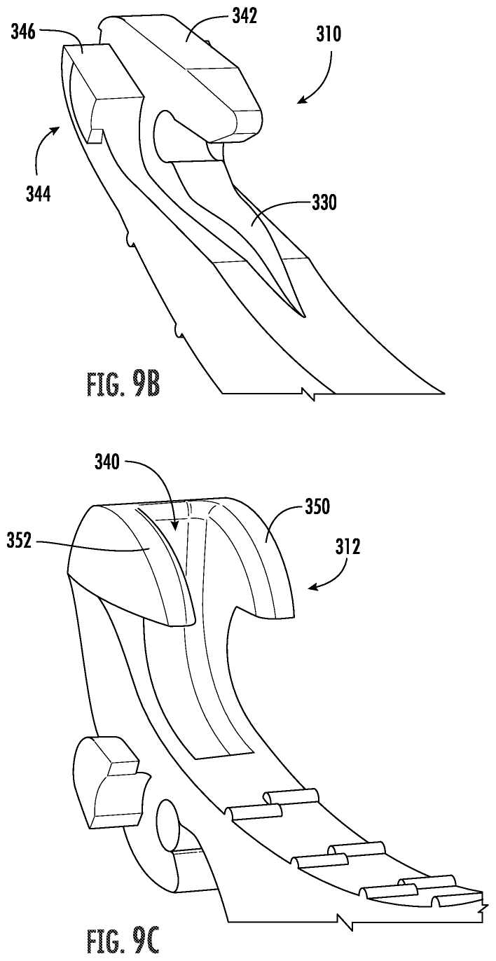

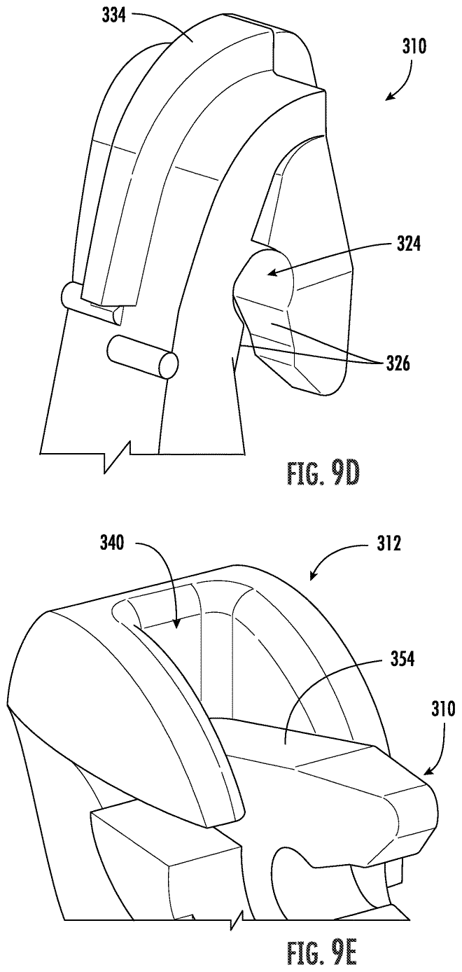

Referring now to FIGS. 8A-9E, a surgical clip 300 will now be described. Clip 300 includes first and second arms 302, 304 pivotally coupled to each other about a pivot point or hinge 306 for movement between an open position (FIG. SA) and a closed position (FIG. 8B). Hinge 306 is preferably a living or integral hinge that comprises an opening 308 that creates two thinned pieces connected to arms 302, 304 to create a flexure bearing that allows arms 302, 304 to open and close. In certain embodiments, clip 300 is naturally biased towards the open position and configured to be closed by the force of jaws 111, 112, as discussed below. In other embodiments, clip 300 may be naturally biased towards the closed (but not latched) position and configured to be opened and then closed and latched by jaws 111, 112.

In certain embodiments, surgical clip 300 comprises a polymer material, such as a non-absorbable polymer or a resorbable or biodegradable polymer. Suitable materials for clip 300 include polyoxymethylene (POM), polyester, nylon, polyetheretherketone (PEEK), polyglycolic acid (PGA or PLGA), poly-L-lactic acid (PLLA), polyethylene (PE) or copolymers thereof. In a preferred embodiment, clip 300 comprises POM.

Surgical clip 300 may be designed, for example, to ligate vessels in a patient. In certain embodiments, clip 300 is sized to ligate vessels having a diameter of about 1 mm to about 10 mm. In certain embodiments, the clip 300 is designed with a sufficient length, strength, and rigidity to ligate medium to large sized vessels, or vessels of up to 10 mm in diameter.

Clip 300 has been designed to eliminate the need for laterally protruding bosses and, therefore, has a thinner profile than conventional polymer clips. The maximum lateral width of clip 300 is less than about 2.0 mm, or about 0.6 mm to about 1.5 mm, or preferably about 0.8 mm to about 1.1 mm (conventional polymer clips designed to ligate vessels up to 10 mm in diameter typically have a maximum lateral width of 2.0 mm or greater). This may allow the user to place the clips in closer proximity to each other and/or place more clips within a target location on the patient, e.g., to provide improved access to the target site.

Of course, it will be recognized that the specific dimensions for the maximum lateral width of clip 300 will vary based on the function of clip. If clip 300 is, for example, designed to ligate smaller vessels (i.e., vessels having diameters of less than 3 mm), than the width of clip will be less than the dimensions described above. However, the overall length/width ratio of clip 300 will remain higher than conventional polymer clips.

First arm 302 includes a latch 310 and second arm 304 includes a hook 312 such that clip 300 can be compressed into a latched or locked position around a grasped vessel or other grasped tissue. In some embodiments, first and second arms 302, 304 include grip features or protrusions 314 extending on the vessel side of each arm. Protrusions 314 are preferably spaced from each other along each arm and provide gripping surfaces to secure clip 300 to the vessel once it is locked in the closed position. These gripping surfaces may also resist axial displacement of the clip.

Referring now to FIGS. 9B and 9C, latch 310 includes a main body 342 sized to slide within a slot 340 in hook 312 that is defined by first and second arms 350, 352. Latch 310 further includes a locking protrusion 344 extending laterally outward from main body 342 that includes a shelf 346. As latch 310 is compressed against hook 310 by jaws 111, 112, the force applied is sufficient to temporarily deform hook 310 backward away from latch 310. This allows locking protrusion 344 to pass below slot 340 in hook 310. Once that has occurred, hook 310 will return to its original location such that protrusion 344 is below slot 340 and shelf 346 engages a lower surface 348 (see FIG. 9A) of one of arms 350, 352. This secures latch 310 to hook 312 and provides both visual and audible confirmation to the user that the latch 310 is now secured to the hook 312.

Clip 300 also includes one or more centering features for aligning latch 310 with hook 312 when the clip is closed 300 by jaws 111, 112. As shown in FIG. 9D, latch 310 includes a rib 334 extending from an internal or vessel-side surface of main body 342 of latch 310. Rib 334 extends downward along the vessel-side surface of latch 310 and is configured to engage the surfaces partially surrounding slot 340 in hook 312 (see FIG. 9C). Rib 334 aligns latch 310 with hook 312 during the process of latching with the instrument to ensure that clip 300 is in the correct position to lock. This configuration allows for a thinner profile clip as it eliminates the need for protruding bosses, as typically found in conventional polymer clips.

Referring to FIGS. 8A and 9A, latch 310 on first arm 302 includes an engagement member 320 for removably coupling to engagement element 164 of drive member 130 and hook 312 on second arm 304 includes an engagement member 322 for removably coupling to engagement element 166 of drive member 130 (see FIG. 15A). Engagement members 320, 322 preferably allow engagement elements 164, 166 to secure drive member 130 to clip 300 during advancement of the clip 300 through wrist 140 into jaws 111, 112 and to control and retain clip 300 throughout the opening and closing of jaws 111, 112 (and consequently the opening and closing of clip 300), as well as any other articulation of end effector 110 relative to shaft 105 (i.e., roll, yaw or pitch movements of the end effector). At the same time, engagement features 320, 322 are designed to release engagement elements 164, 166 upon sufficient application of force to the drive member 130. As discussed below, this allows the user to remove drive member 130 from clip 300 after the clip 300 has been closed onto a vessel.

One particular advantage of this feature is that the captured drive member 130 within jaws 111, 112 allows the drive member 130 and/or jaws 111, 112 to pull clip 300 open while it is disposed within the jaws. Conventional polymer clips tend to creep over time when stored in the clip cartridge (i.e., move into a more closed position). This prevents the clips from springing themselves open after they have been advanced into the jaws (as typically occurs with conventional polymer clips). This feature also facilitates relocation of the main locating “boss” features from the clip to the drive member, which allows for the design of a clip having a thinner profile than conventional clips (discussed in more detail below). In addition, this feature allows the joint between the engagement elements 164, 166 of drive member 130 and engagement features 320, 322 of clip 300 to rotate while jaws 111, 112 open and close.

In one embodiment, engagement features 320, 322 each comprise a snap fit feature that comprises a cutout or opening 324 sized to accommodate shaft 174 of engagement elements 164, 166 and protrusions 326 on either side of openings 324 that create a reduced-diameter entry to the openings 324 (see FIGS. 9A and 9D). This allows shafts 174 of engagement elements 164, 166 of drive member 130 to be advanced into openings 324 with a sufficient application of force (discussed below). At the same time, shafts 174 will remain secured within openings 324 until a sufficient withdrawal force is applied to drive member 130.

In certain embodiments, first and second arms 302, 204 include tapered ribs 330 extending towards the non-vessel side of the arms (see FIGS. 8C, 8D, 9A and 9B). These ribs 330 taper in two directions (i.e., laterally and vertically) to provide lateral and vertical guide features for drive member 130 to align engagement elements 164, 166 of drive member 130 with each clip 300. Specifically, ribs 330 taper inwardly from each lateral side of ribs 330 in the proximal direction to provide lateral alignment. In addition, ribs 330 taper towards arms 302, 304 in the proximal direction to provide vertical alignment. This allows the drive member 130 to center and/or align itself on clip 300 during engagement within cartridge 120.

Cip 300 has been designed such that the force required to remove latch 310 from hook 312 after it has been latched thereto is greater than the force required to remove drive member 130 from clip 300 (i.e., the latch mechanism is stronger than the engagement mechanism). Thus, locking protrusion 344 of latch 310 secures the latch 310 to hook 312 as drive member 130 is withdrawn proximally and engagement elements 164, 166 are withdrawn from engagement members 322, 320 of clip 300.

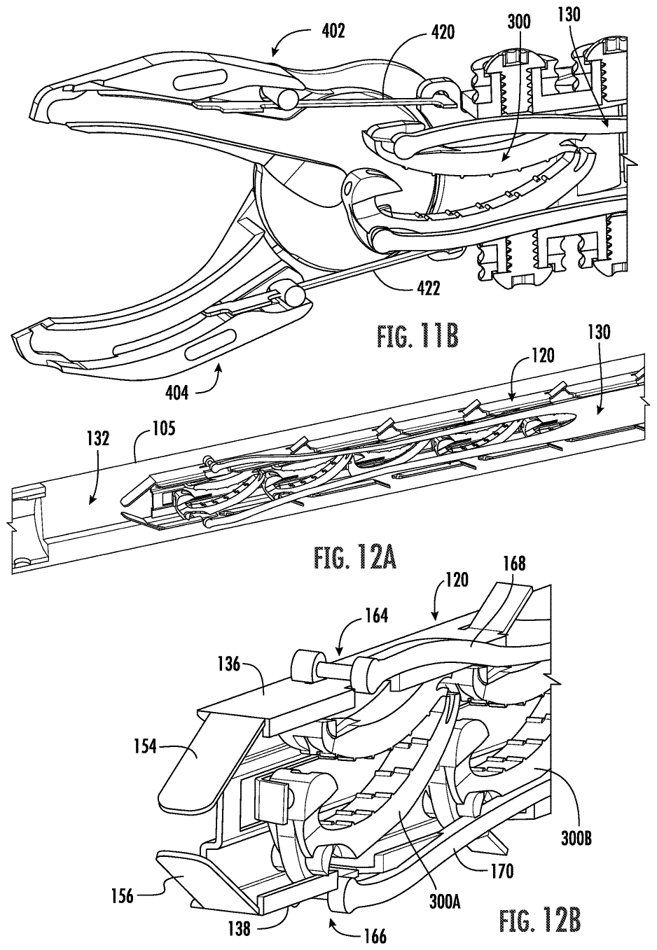

Clip 300 further includes a protrusion 332 extending from the side of hook 312 that facilitates guidance of clip 300 through a guide track 442 of jaw 404 as clip 300 is advanced into the jaws (see FIG. 11A). In one embodiment, locking protrusion 344 on latch 310 is configured to also function as a guide protrusion that advances through guide track 440 of jaw 402. Protrusions 332, 344 may also function to engage tracks 440, 442 of jaws 402, 404 if drive member 130 becomes disengaged with clip 130 during advancement, thereby preventing premature disengagement of the clip 300 from jaws 111, 112. These protrusions 332 are preferably sized to be thinner than the boss protrusions on conventional clips.

Clip 300 also includes an anti-scissoring feature that ensures that latch 310 remains aligned with hook 312 after they are locked together. This feature includes a fin 354 extending on the upper surface of main body 342 of latch 312. When latch 310 is locked to hook 312, fin 354 is trapped within slot 340 of hook 312, which prevents any scissoring motion that could cause disengagement of the latch from the hook (see FIG. 9E). Providing a fin 354 that fits within a slot 340 allows for the design of a thinner profile clip than conventional clips that typically use boss-like projections around the hook to mitigate scissoring.

Referring now to FIGS. 10A-10E, one embodiment of a jaw assembly 400 for instrument 100 will now be described. As shown, jaw assembly 400 comprises first and second jaws 402, 404 that are pivotally coupled to each other at a hinge joint 406. First and second jaws 402, 404 are also capable of articulating together about an axis substantially perpendicular to the longitudinal axis (e.g., the pitch axis), as shown in FIG. 11A. In addition, first and second jaws 402, 404 are designed to move relative to each other between an open position (as shown in FIG. 10A and FIG. 10B) and a closed position, wherein the distal ends of the jaws are near, or in contact, with each other (see FIG. 10C). In the preferred embodiment, both jaws 402, 404 are movable jaws, although it will be recognized that one of the jaws may be a movable jaw configured to move between open and closed positions relative to the other jaw.

In one preferred embodiment, hinge 406 comprises a first link 408 and a second link 410 on one side of jaw assembly 400 and a third link 412 on the other side of jaw assembly 400 (see FIG. 10D). First link 408 comprises a slot pin 414 configured to slide through a slot 415 of first jaw 402 and a pin or screw 419 coupled to a first pulley 421. Similarly, second link 410 comprises a slot pin 416 configured to slide through a slot 417 of second jaw 404 (see FIG. 10B) and a pin or screw 423 that is coupled to first pulley 421.

As shown in FIG. 10D, third link 412 is positioned on the other side of jaws 402, 404 and includes a slot pin 425 configured to slide through a slot 427 of first jaw 402 on the other side of slot 415. In the preferred embodiment, slot pin 425 is the same slot pin as slot pin 414 and extends completely through jaw 402 from first link 408 to third link 412. Third link 411 has another pin or screw 429 coupled to a second pulley 431 opposite first pulley 419.

In one embodiment, jaw assembly 400 includes a pulley and linkage system that is based on a single axle, in which both jaw articulation and wrist yaw are rotated. The single pivot helps to minimize gaps that may form between sections of the linkage that can make it more difficult to advance clips into jaws 402, 404. The slots in the jaws are pushed on by an axle in the corner of a four bar linkage. Each jaw has its own four bar linkage that is substantially the same (but reversed) that spans between the two pulleys and acts as a differential. As shown in FIG. 10D, pulley 419 acts as the first “link” in the four bar linkage and, first link 408 acts as the second “link”, third link 412 acts as the “third link” and second pulley 431 acts as the fourth link. The slot pin 414, 425 is the “pivot” between the second and third links of the four-bar linkage.

When the two pulleys are driven together in the same direction, jaws 402, 404 will rotate in the yaw direction relative to shaft 105 together. Any differential motion between the pulleys, however, will drive the linkages to move the jaws relative to each other (i.e., open and close). The linkages may also be disposed close to the point of the links scissoring so that they amplify the force as the clip is closing (similar to a vise grip). A more complete description of this feature can be found in commonly assigned, co-pending US Provisional Application, filed concurrently with this application (Attorney Docket No. P06660-US-PRV).

Referring again to FIG. 11A, jaw assembly 400 comprises first and second ribbons, bands, wires, or cables 420, 422 extending from wrist assembly 140 to first and second jaws 402, 404, respectively. Ribbons 420, 422 preferably comprise a flexible material, such as nitinol, spring steel or the like, such that ribbons 420, 422 bend or flex when jaws 402, 404 are articulated about the jaw axis. Ribbons 420, 422 each have a proximal end 426, 428 coupled to wrist assembly 140 and a distal end 430, 432 extending into each of first and second jaws 402, 404. In certain embodiments, the distal ends 430, 432 may be secured to, or otherwise coupled to jaws 402, 404. In other embodiments, the ribbons 420, 422 extend on the inside of slot pins 416, 414 and have sufficient rigidity to remain in place within jaws 402, 404.

As shown in FIGS. 11A and 11B, ribbons 420, 422 function to contain drive member 130 and clip 300 when these components have been driven into jaws 402, 404 and the jaws are articulated about the yaw axis of the instrument. More specifically, distal advancement of drive member 130 (and clip 300 therewith) passes between ribbons 420, 422 even when jaws 402, 404 are articulated relative to the longitudinal axis of the instrument 100 (see FIG. 11A).

As show in FIGS. 11A and 18, first and second jaws 402, 404 each include guide tracks 440, 442 that generally extend from a proximal portion of the jaws to the distal end 434, 436 of each jaw. Guide tracks 430, 432 generally extend inside of ribbons 420, 422. Guide tracks 430, 432 and ribbons 420, 422 ensure that arms 168, 170 of drive member 130 pass along guide tracks 430, 432 to distal ends 434, 436 as drive member 130 is advanced distally into jaws 402, 404.

Referring now to FIG. 10E, jaws 402, 404 may each include an engagement feature at their distal ends to secure the clips therein after they have been delivered by drive member 130. In one embodiment, the engagement features comprise ramped leaf springs 437 positioned on either side of guide tracks 440, 442. As shown, guide tracks 440, 442 taper inwardly in the distal direction such that the lateral width across guide tracks 440, 442 decreases distally. As engagement elements 164, 166 of drive member 130 and the clip advance distally through guide tracks 440, 442, they contact the outer surfaces 439, 441 of guide tracks 440, 442, which become narrower as the clip is advanced distally. The clip and engagement elements 164, 166 press against leaf springs 437 so that they are biased outwardly to allow the clip and engagement elements 164, 166 to move to the distal ends of the jaws. This spring pressure applied inwardly by leaf springs 437 retains the clip and engagement elements 164, 166 within the distal ends of the jaws and inhibits them from withdrawing proximally and/or falling out of the jaws.

As shown in FIGS. 10, 19B and 20B, jaws 402, 404 each have distal end portions 434, 436 that include a cutout 454. Cutouts 454 are disposed at the distal end of guide tracks 430, 432. Cutouts 454 preferably have a larger cross-sectional area than track guide tracks 430, 432. This ensures that the distal ends of latch 310 and hook 312 of clip 300 have sufficient clearance as they are coupled to jaws 402, 404 (as these elements are generally distal of engagement elements 164, 166 of drive member 130 as clip 300 is advanced distally into the jaws 402, 404. In addition, cutouts 454 facilitate removal of the clip 300 from jaws 402, 404 when clip 300 is closed and latched and drive member 130 has been decoupled from clip 300.

In one embodiment shown in FIG. 19B, cutouts 454 each include a longitudinal component 456 for receiving the hook 312 and latch 310 of clip 300 and a horizontal component 448 for receiving engagement elements 164, 166 of drive member 130. Horizontal components 448 are sized and configured to contain engagement elements 164, 166 within jaws 402, 404 (i.e., they prevent the engagement elements 164, 166 from passing distally of jaws 402, 404). In certain embodiments, horizontal components 448 have a lateral span that is less than the overall lateral span of engagement elements 164, 166 (i.e., from one end of outer shaft 172 to the other end of outer shaft 176). In other embodiments, horizontal components 448 have a longitudinal span that is smaller than the diameter of outer shafts 172, 176 of engagement elements 164, 166. In certain embodiments, both the longitudinal and lateral span of horizontal components 448 are small enough to contain engagement elements therein.

Referring now to FIGS. 4, 5 and 12A-21, a method for applying multiple clips to tissue or vessels in a patient will now be described. As shown in FIGS. 4 and 5, drive member 130 is generally positioned along the side of cartridge 120 such that engagement elements 164, 166 are located distal of tabs 154, 156 on the distal end of cartridge 120. To engage a clip 300 with the drive member 130, drive member 130 is withdrawn proximally such that engagement elements 164, 166 slide along tabs 154, 156 and spread out arms 168, 170 of drive member 130 such that engagement elements 164, 166 move along upper and lower surfaces 136, 138 of cartridge 120 (see FIGS. 12A and 12B).

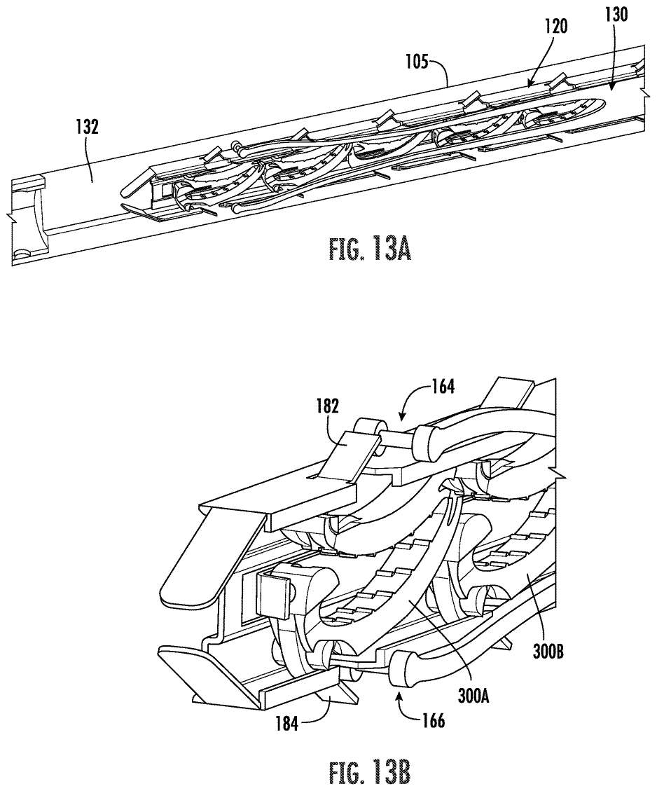

Referring now to FIGS. 13A and 13B, as drive member 130 is withdrawn proximally, engagement elements 164, 166 slide over tabs 182, 184 of the first clip 300 within cartridge 130. In some embodiments, tabs 182, 184 are configured to spring inwardly to facilitate the movement of engagement elements 164, 166 over the tabs 182, 184. In other embodiments, arms 168, 170 are stretched further outward to allow this movement.

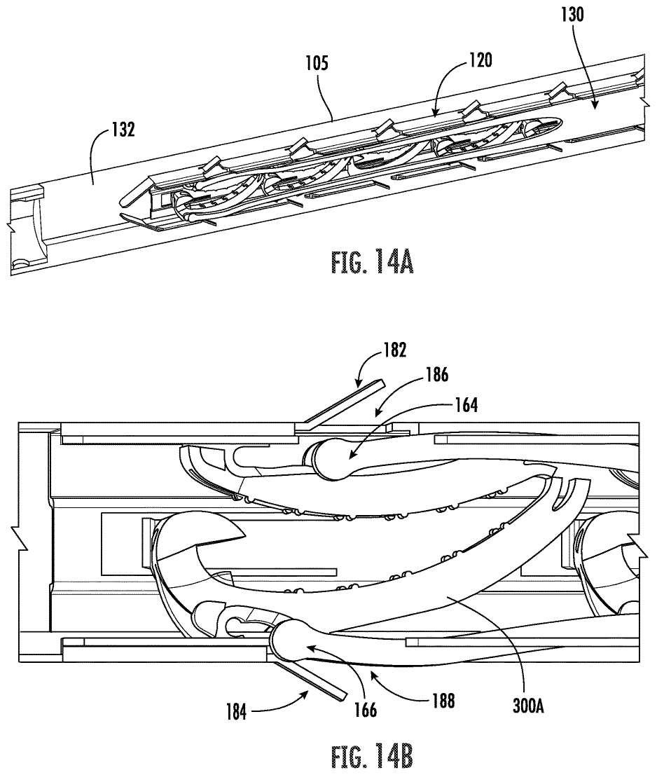

Referring now to FIGS. 14A and 14B, once engagement elements 164, 166 are proximal of tabs 182, 184, they will enter openings 186, 188 into the interior of cartridge 130. In some embodiments, this movement will occur automatically as engagement elements 164, 166 pass proximally of tabs 182, 184. In other embodiments, drive member 130 may be advanced distally to move engagement elements 164, 166 into openings 186, 188. Upper and lower tabs 182, 184 will generally direct engagement elements 164, 166 downward into cartridge 120.

As shown in FIGS. 15A-15C, drive member 130 is then moved further distally until engagement elements 164, 166 engage with engagement features 320, 322 of the first clip 300A (see also FIG. 9). More specifically, the inner shaft 174 of each element 162, 164 passes through the snap-fit design of features 320, 322 by passing through protrusions 326 and into openings 324 of the latch 310 and hook 312 of clip 300. As drive member 130 is moved further distally, retaining tab 180 is flexed away such that clip 300A is released from cartridge 120. At this point, the first clip 300A is coupled to drive member 130 and no longer secured within cartridge 120 such that drive member 130 can move clip 300 into end effector 110. The proximal clip 300B remain secured within cartridge 120.

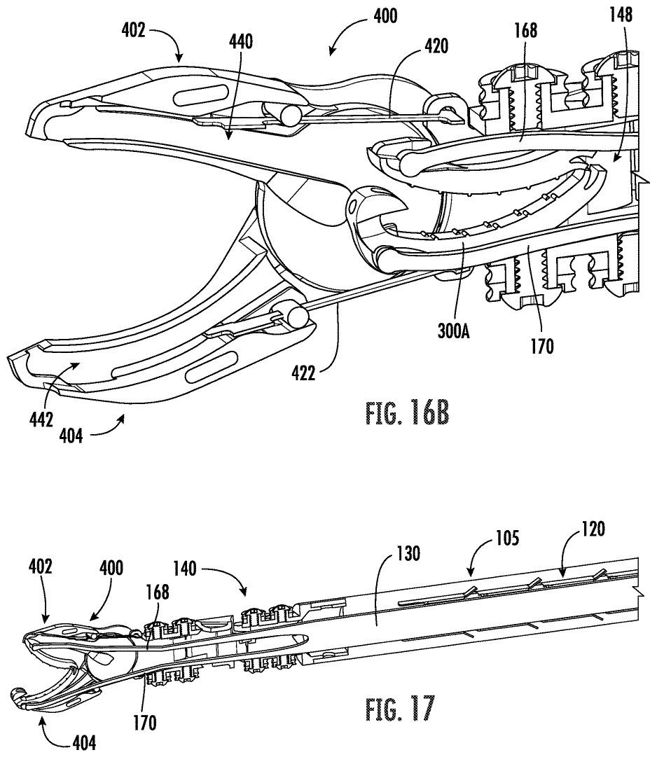

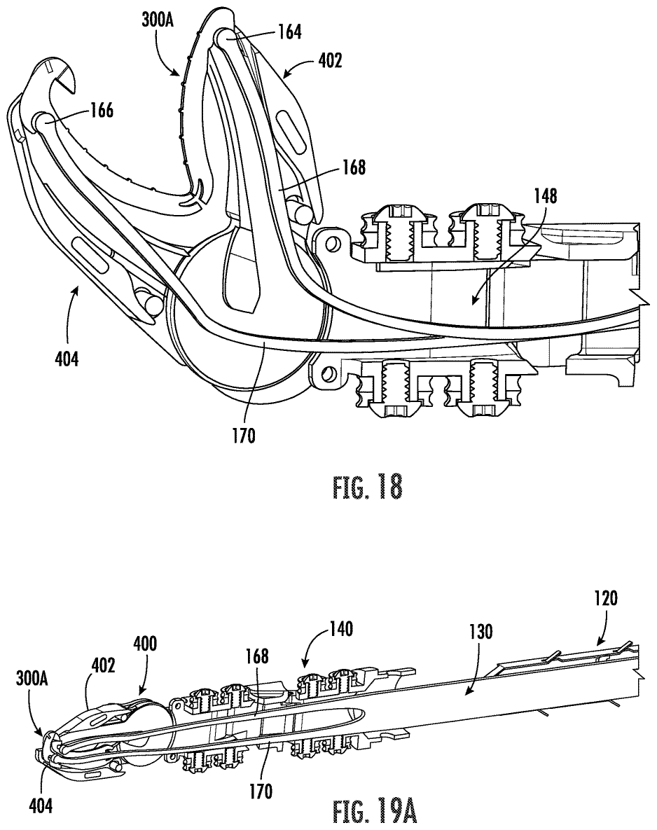

Referring now to FIGS. 16A and 16B, drive member 130 advances clip 300 through central lumens 146, 148 of within wrist assembly 140 and into end effector 110. As arms 168, 170 of drive member 130 enter the jaw assembly 400, ribbons 420, 422 constrain the movement of arms 168, 170 such that the arms 168, 170 enter guide tracks 440, 442 of first and second jaws 402, 404 (see FIGS. 17 and 18). Drive member 130 is advanced distally until engagement elements 164, 166 (and hook 312 and latch 310 of clip 310A) engage cutouts 454 in distal end portions 434, 436 of jaws 402, 404 (see FIGS. 19A and 19B).

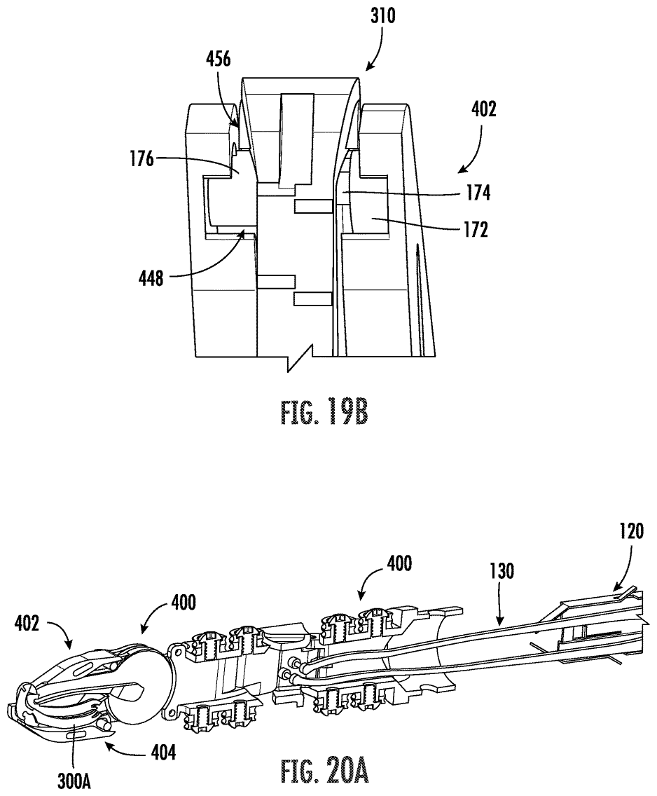

Referring now to FIGS. 20A and 20B, when the surgeon has positioned the first clip 300A in the desired location to clamp onto tissue or a vessel, jaws 402, 404 are closed. As noted above, jaws 402, 404 provide sufficient force to close clip 300A and to secure latch 310 into hook 312. Once that has occurred, drive member 130 may be withdrawn proximally by exerting sufficient force onto element 130 to withdraw engagement elements 162, 164 of drive member 130 from engagement elements 320, 322 of latch 310 and hook 312, respectively. Once disengagement of drive member 130 from clip 300A, drive member 130 may be withdrawn proximally back through wrist assembly 400 and into shaft 105 of instrument (see FIG. 20A).

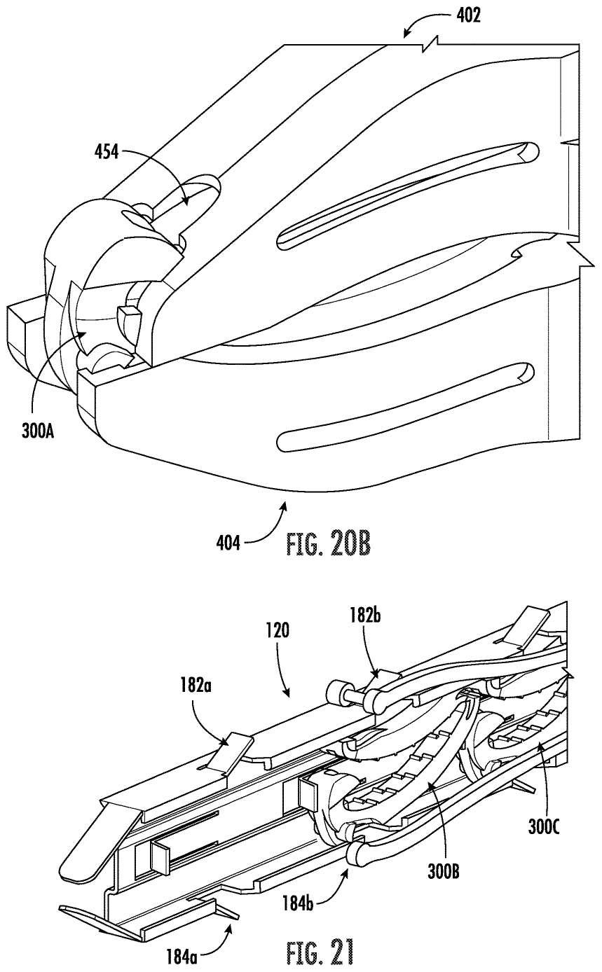

Referring now to FIG. 21, to engage a second clip 300B from cartridge 120, engagement elements 164, 166 of drive member 130 are withdrawn over distal tabs 154, 156 and past first upper and lower tabs 182a, 184a and second upper and lower tabs 182b, 184b to the second clip 300B. The process may be then repeated to advance second clip 300B to the jaws of the end effector, then a third clip 300C, etc.

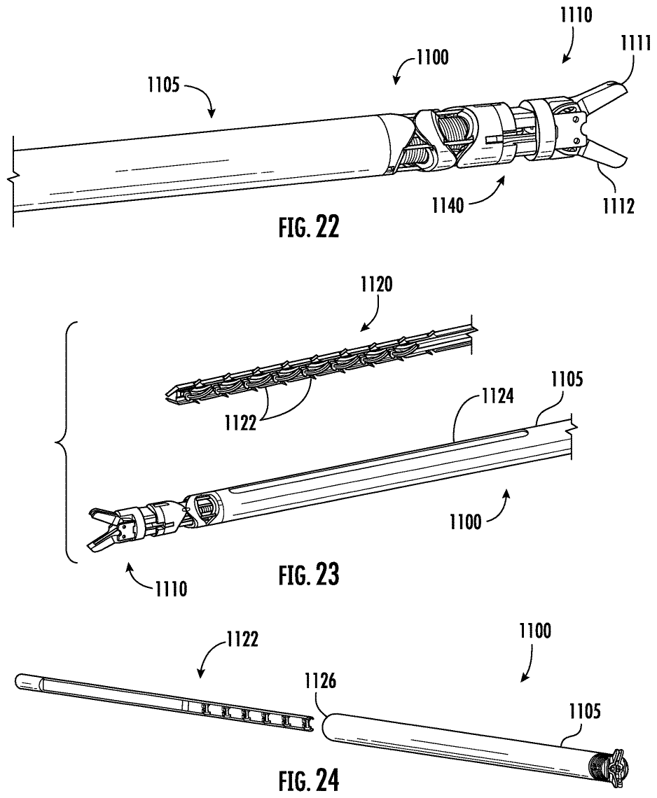

FIG. 22 illustrates the distal end portion of an alternative embodiment of a surgical instrument 1100 in accordance with an illustrative embodiment. Surgical instrument 1100 includes an end effector 1110, an elongated shaft 1105 and a wrist assembly 1140 coupling end effector 1110 to shaft 1105. The proximal end portion of elongate shaft 1105 is operatively connected to an actuation mechanism (not shown), although as those skilled in the art reading this description will appreciate, components of the actuation mechanism may extend into, and/or pass through elongated shaft 1105 and/or wrist assembly 1140.

End effector 1110 includes a first jaw 1111 and a second jaw 1112 c configured to move between an open position (as shown in FIG. 22) where the jaws are spaced apart from one another and a closed position to force the jaws into compressing contact with the legs of a clip to close and seal the clip around vessels or tissue. In certain embodiments, second jaw 1112 is a movable jaw configured to move from an open position to a closed position relative to first jaw 1111. In other embodiments, first jaw 1111 is a movable jaw configured to move between open and closed positions relative to second jaw 1112. In still other embodiments, both jaws 1111, 1112 are movable relative to each other.

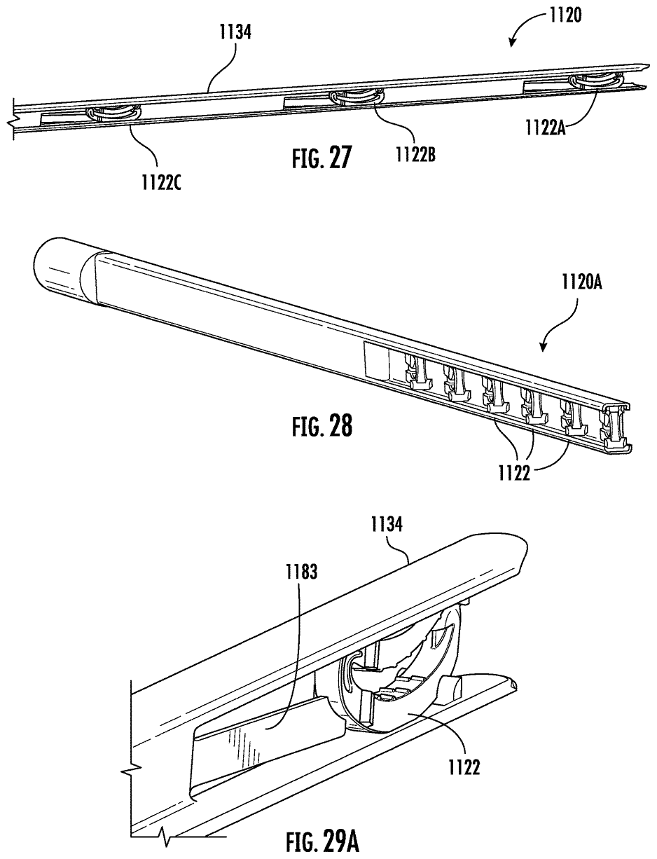

Referring now to FIGS. 23 and 24, instrument 1100 may be provided with a clip cartridge 1120 that comprises a plurality of surgical clips 1122 and is installed into surgical instrument 1100. In certain embodiments, cartridge 1120 may be installed through a longitudinal slot 1124 in the side of shaft 1105 (FIGS. 23 and 27). In other embodiments, a cartridge 1120A may be installed through an opening in a proximal end 1126 of shaft (FIGS. 24 and 28). Cartridge 1120 may contain between about 1 to 20 clips, preferably between about 2 to 12 clips. Clip 1122 preferably extend in a substantially parallel direction relative to the longitudinal axis of shaft 1105.

Cartridge 1120 may be constructed from any suitable materials known in the art, such as a single-molded plastic body or sheet metal. Cartridge 1120 may be adapted to accommodate any suitable desired sizes and configurations of clips 1122, including conventional clips (e.g., titanium, tantalum, or stainless steel ligation clips, such as the Horizon™, Hemoclip® or the like and/or polymer clips, such as the Vas-Q-Clip®, the Weck® Hem-o-lok® or the like). Alternatively, cartridge 1120 may be adapted to accommodate the novel clips 1300 described herein and shown in FIGS. 8A-9D and FIGS. 34-39B.

Wrist assembly 1140 is positioned between end effector 1110 and elongated shaft 1105. Wrist assembly 1140 may provide a desired amount of motion, such as +/−90 degrees in a pitch, yaw and/roll direction (discussed in further detail below). Cables or other actuators (not shown) are drivingly coupled with the wrist assembly 1140 and actuated to impart motion to wrist assembly 1140.

In certain embodiments, drive member 1130 is coupled to instrument shaft 1105 such that drive member 1130 is included as part of the overall instrument 1100, which may be constructed of materials designed for re-use of the instrument in multiple surgical procedures. In other embodiments, drive member 1130 is coupled to cartridge 1120 such that drive member 1130 is included as part of the clip cartridge 1120, which may be constructed of materials designed for disposable or single-use applications. In either embodiment, drive member 1130 is configured for longitudinal displacement relative to shaft 1105 to advance clips 1122 from cartridge 1120 to jaws 1111, 1112 of end effector 1110, as discussed in more detail below.

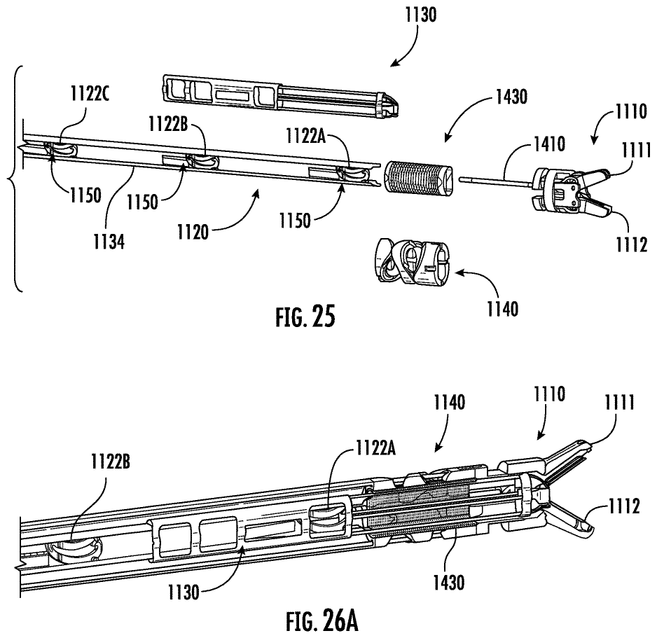

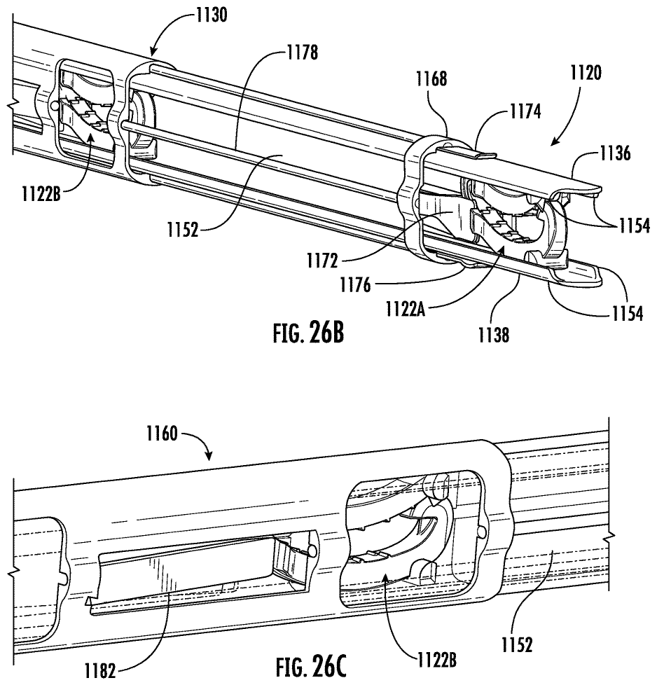

As shown in FIGS. 25, 26A and 26B, clip cartridge 1220 comprises a housing 1134 having upper and lower walls 1136, 1138 for retaining a plurality of clips 1122 within housing 1134. Housing 1134 further comprises a series of internal chambers 1150 for housing each clip (labeled 1122A, 1122B and 1122C in FIG. 26A) within housing 1134. Internal chambers 1150 are preferably spaced substantially equally from each other and the distal-most chamber 1150 that houses the distal-most clip 1122A is preferably spaced from the jaws 1111, 1112 a distance substantially equal to the spacing between the clips. This allows the drive member 1130 to move all of the clips forward distally the same distance, thereby allowing, for example, drive member 1130 to advance clip 1122A to jaws 1111, 1112 while moving clip 1122B to the location previously possessed by clip 1122A, etc. This design increases the speed and efficiency of delivering multiple clips to a target site. Further details of this operation will be discussed below.

Housing 1134 may include one or more longitudinal walls extending between upper and lower walls 1136, 1138. In one embodiment, housing 1134 includes a longitudinal wall 1152 on the opposite side of advancer tabs 1182 of drive member 1130 (see FIG. 26C; discussed in more detail below). Housing 1134 may also have a second longitudinal wall (not shown) on the side adjacent to advanced tabs 1182, or this side may be substantially open (or include windows or openings within the second longitudinal wall) such that clips 1122 can be accessed by advancer tabs 1182 of drive member 1130 from this side of housing 1134, as shown in FIGS. 26B and 26C.

In one embodiment, housing 1134 may further comprise overhang features 1154 extending towards the longitudinal axis from each side of upper and lower walls 1136, 1138 (see FIG. 26B). These overhang features ensure that the clips remain contained within cartridge housing 1134, but still allow distal movement of the clips through housing 1134. The upper and lower overhang features 1154 are preferably spaced from each other a sufficient distance to retain clips 1122 within housing 1134, while allowing advancer tabs 1182 and retainer tabs 1170, 1172 of drive member 1130 to access clips (discussed below).

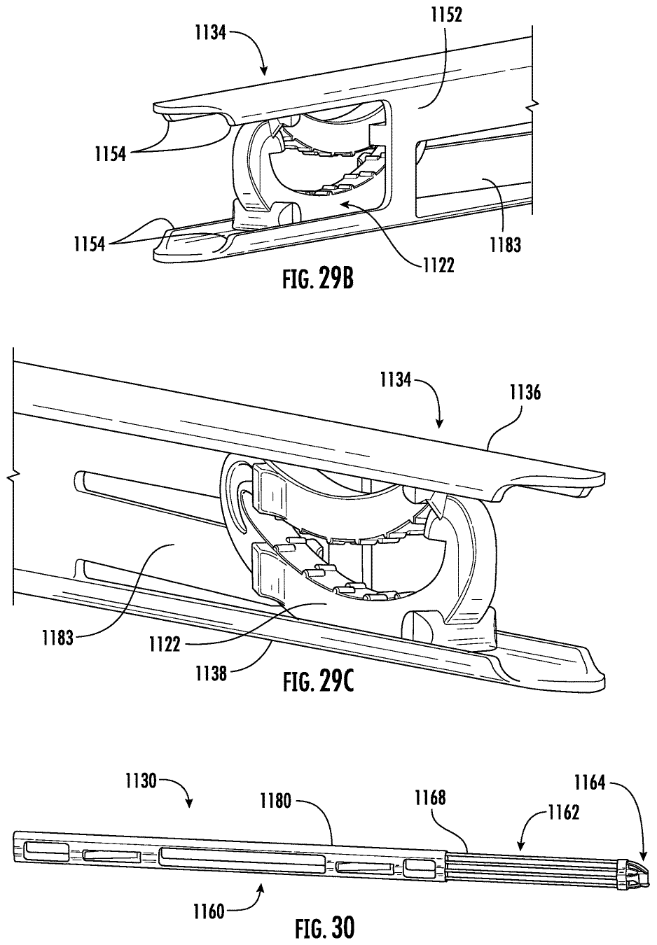

As shown in FIG. 29A, cartridge 1120 includes a series of ratcheting tabs 1183 positioned proximally of each clip 1300. Ratcheting tabs 1183 are biased inwardly and ensure that the clips are not dragged backwards or proximally as the drive member 1130 withdraws proximally to engage another clip. At the same time, ratcheting tabs 1183 define a ramped surface that allows the clips positioned proximal of each ratcheting tab to move distally along the ramp into the next distal-most position to engage with drive member 1130.

As shown in FIGS. 30-32, drive member 1130 comprises a proximal component 1160, a distal component 1164 and a flexible component 1162 coupling proximal component 1160 with distal component 1164. Distal component 1164 is generally configured to removably couple to one or more of the surgical clips 1122 in cartridge 1120 (discussed below). Proximal component 1160 is configured to extend through shaft 105, and may have one or more proximal interfaces (not shown) for cooperating with an actuation mechanism (not shown) to advance drive member 1130 distally and proximally relative to shaft 1105.

Flexible component 1162 preferably comprises a material that is sufficiently rigid to have enough compressive strength to push through wrist assembly 1140 into jaws 1111, 1112. At the same time, flexible component 1162 comprises a material that is flexible and resilient enough to bend as end effector 1110 is articulated relative to shaft 1105 at wrist assembly 1140. In a preferred embodiment, flexible component 1162 comprises nitinol, polymers, such as PEEK, spring steel or similar materials.

In one embodiment, flexible component 1162 comprises a plurality of rods 1168 extending between proximal and distal components 1160, 1164 and having a length at least as long as the wrist assembly 1140. Rods 1168 are configured to bend as wrist assembly 1140 articulates end effector 1110 relative to shaft 1105 such that distal component 1164 of drive member 1130 may be positioned within end effector 1110 as the wrist assembly articulates 1140. This allows the drive member to position clips 1122 within the jaws 1111, 1112 of the end effector 1110 and to retain the clip 1122 while jaws 1111, 1112 are opened and closed and/or articulated relative to the shaft of the instrument. Thus, the surgeon may fully open the clips after they have been advanced into the jaws so that they can be effectively positioned around a target vessel or tissue. In addition, this allows the surgeon to reposition the jaws relative to the shaft after the clip has been advanced into the jaws.

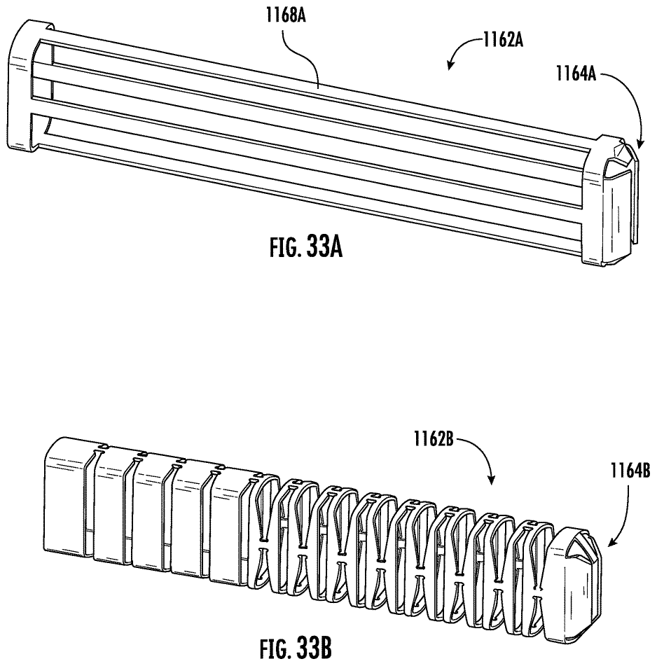

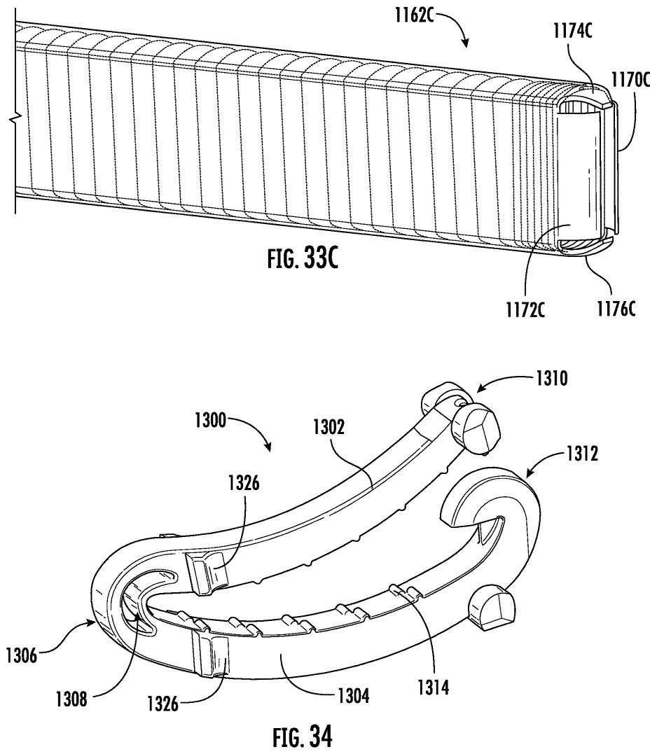

FIGS. 33A-33C illustrates alternative embodiments of a flexible component of drive member 1130. As shown in FIG. 33A, a flexible component 1162A comprises of a plurality of straps 1168A extending between the distal and proximal components of drive member 1130. Straps 1168 may be any suitable shape, such as circular, rectangular, square or the like. In one embodiment, straps 1168 are substantially rectangular and comprise nitinol, a stainless steel spring or similar material.

FIG. 33B illustrates another embodiment of flexible component 1162B that comprises laser cut tubing to form an accordion-type shape that allows for bending of flexible component 1162B relative to distal and proximal components of drive member 1130. FIG. 33C illustrates another embodiment of flexible component 1162C that comprises a flexible catheter-like construction formed of a reinforced polymer jacket material. The retainer tabs 1170C, 1172C and the flexible tabs 1174C, 1176C may be, for example, formed by cutting out the jacket material.

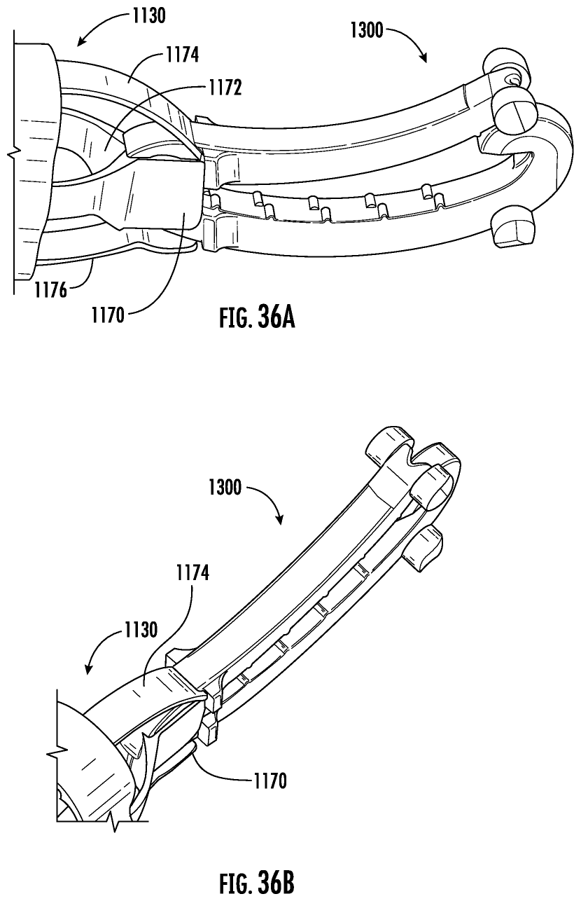

Referring now to FIG. 31, distal component 1164 of drive member 1130 comprises an engagement element for removably coupling drive member 1130 to the surgical clip within cartridge 1120. In one embodiment, the engagement element comprises first and second retainer tabs 1170, 1172 extending distally from drive member 1130. Retainer tabs 1170, 1172 are located on the lateral sides of drive member 1130 and are preferably biased inwardly towards the longitudinal axis of the shaft 1105 with sufficient force to retain and control a clip 1122 that is held within tabs 1170, 1172 (see, for example, FIGS. 36A and 36B).

In certain embodiments, distal component 1164 further includes upper and lower retainer tabs 1174, 1176 extending distally from drive member 1130 and spaced from each other above and below retainer tabs 1170, 1172. Retainer tabs 1174, 1176 are located on the upper and lower portions of drive member 1130 and are biased inwardly to provide additional security to the coupling of drive member 1130 to the clip 1122 (in addition to tabs 1170, 1172). Tabs 1174, 1176 may also function to guide drive member 1130 within clip cartridge 1120 by flexing upwards and downwards as the drive member 1130 is withdrawn proximally into housing 1134 of cartridge 1120 (see FIG. 26B).

Distal component 1164 also includes an annular collar 1178 that provides structure for retainer tabs 1170, 1172, 1174 and 1176. Collar 1178 is sized to slide around cartridge housing 1120. In addition, collar 1178 is sized to fit through internal tube 1430 of wrist assembly 1140 (discussed in more detail below).

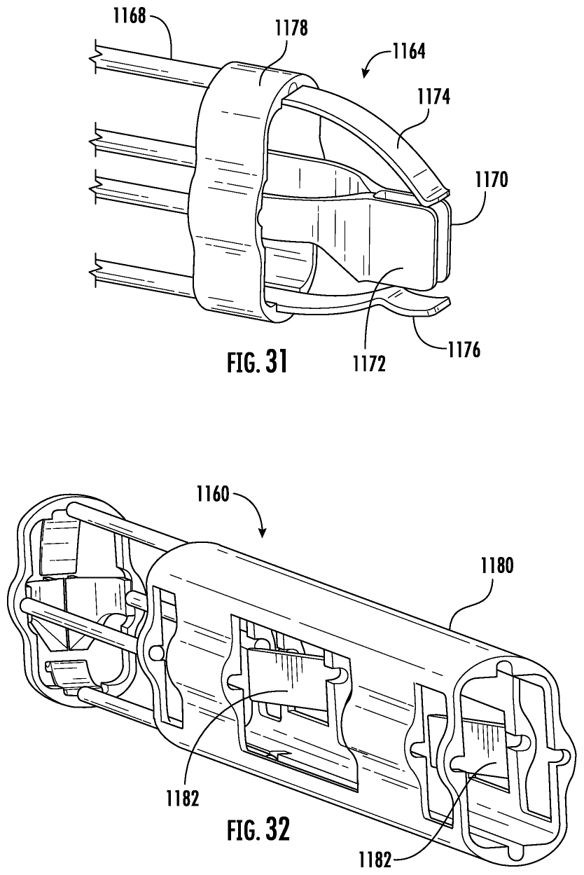

Referring now to FIGS. 26C and 32, proximal component 1160 of drive member 1130 comprises a structural frame 1180 sized to slide around the housing 1134 of clip cartridge 1120. Proximal component 1160 further includes a series of advancer tabs 1182 on both sides of cartridge 1120 (see also FIG. 32) that are biased inwardly towards the longitudinal axis of the shaft. Advancer tabs 1182 are configured to snap inwardly behind the proximal clips 1122B, 1122C, etc. within cartridge 1120 such that distal advancement of drive member 1130 also advances the proximal clips at the same time as the distalmost clip 1122A is advanced into the jaws 1111, 1112. As discussed above, the clips are equally spaced from each other within cartridge 1120 such that advancement of distal clip 1122A into the jaws 1111, 1112 also causes advancement of the next proximal clip 1122B to the previous location of distal clip 1122A. This places clip 1122B in position to be engaged by retainer tabs 1170, 1172 when drive member 1130 is withdrawn proximally after releasing from the distal clip 1122A (discussed below).

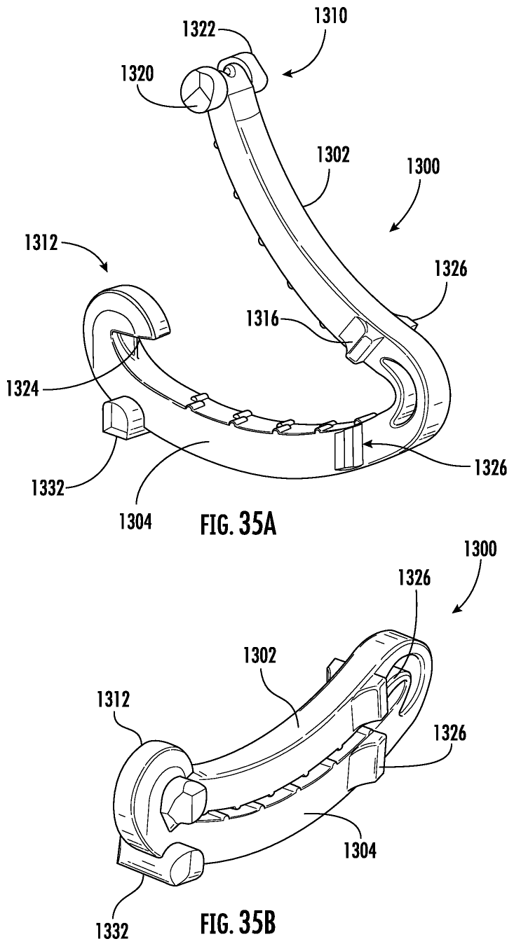

Referring now to FIGS. 34-39C, various embodiments of a surgical clip 1300 will now be described. As shown in FIGS. 34-36B, on embodiment of a clip 1300 includes first and second arms 1302, 1304 pivotally coupled to each other about a pivot point or hinge 1306 for movement between an open position (FIG. 35A) and a closed position (FIG. 35B). Hinge 1306 is preferably a living or integral hinge that comprises an opening 1308 that creates two thinned pieces connected to arms 1302, 1304 to create a flexure bearing that allows arms 1302, 1304 to open and close. In certain embodiments, clip 1300 is naturally biased towards the open position and configured to be closed by the force of jaws 1111, 1112, as discussed below. In other embodiments, clip 1300 may be naturally biased towards the closed (but not latched) position and configured to be opened and then closed and latched by jaws 1111, 1112.

In certain embodiments, surgical clip 1300 comprises a polymer material, such as a non-absorbable polymer or a resorbable or biodegradable polymer. Suitable materials for clip 300 include polyoxymethylene (POM), polyester, nylon, polyetheretherketone (PEEK), polyglycolic acid (PGA or PLGA), poly-L-lactic acid (PLLA), polyehtylene (PE) or copolymers thereof. In a preferred embodiment, clip 300 comprises polyoxymethylene (POM). Surgical clip 1300 may be designed, for example, to ligate vessels in a patient. In certain embodiments, clip 1300 is sized to ligate vessels having a diameter of about 3 mm to about 10 mm.

First arm 1302 includes a latch 1310 and second arm 1304 includes a hook 1312 such that clip 3100 can be compressed into a latched or locked position around a grasped vessel or other grasped tissue. In some embodiments, first and second arms 1302, 1304 include grip features or protrusions 1314 extending on the vessel side of each arm. Protrusions 1314 are preferably spaced from each other along each arm and provide gripping surfaces to secure clip 1300 to the vessel once it is locked in the closed position. These gripping surfaces may also resist axial displacement of the clip.

Referring now to FIGS. 35A and 35B, latch 1310 includes first and second protrusions or bosses 1320, 1322 extend laterally outward from latch 1310. As latch 1310 is compressed against hook 1310 by jaws 1111, 1112, the force applied is sufficient to temporarily deform hook 1310 backward away from latch 1310. This allows locking bosses 1320, 1322 to pass below hook 1312. Once that has occurred, hook 1310 will return to its original location such that bosses 1320, 1322 are below hook 1310 and a lower surface 1324 of hook 1310 engages with bosses to secure latch 1310 to hook 1312 and provides both visual and audible confirmation to the user that the latch 1310 is now secured to the hook 1312.

Clip 1300 may be designed such that the force required to remove latch 1310 from hook 1312 after it has been latched thereto is greater than the force required to remove drive member 1130 from clip 1300 (i.e., the latch mechanism is stronger than the engagement mechanism). Thus, bosses 1320, 1322 of latch 1310 secures the latch 1310 to hook 1312 as drive member 1130 is withdrawn proximally and engagement tabs 1170, 1172 are withdrawn from clip 1300. In an alternative embodiment, the jaws of the instrument include an engagement feature that secures the clip 1300 to the jaws as the drive member 1130 is withdrawn proximally and releases from the clip (discussed in more detail below).

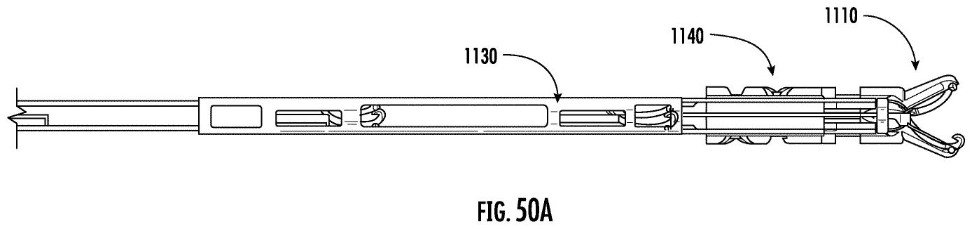

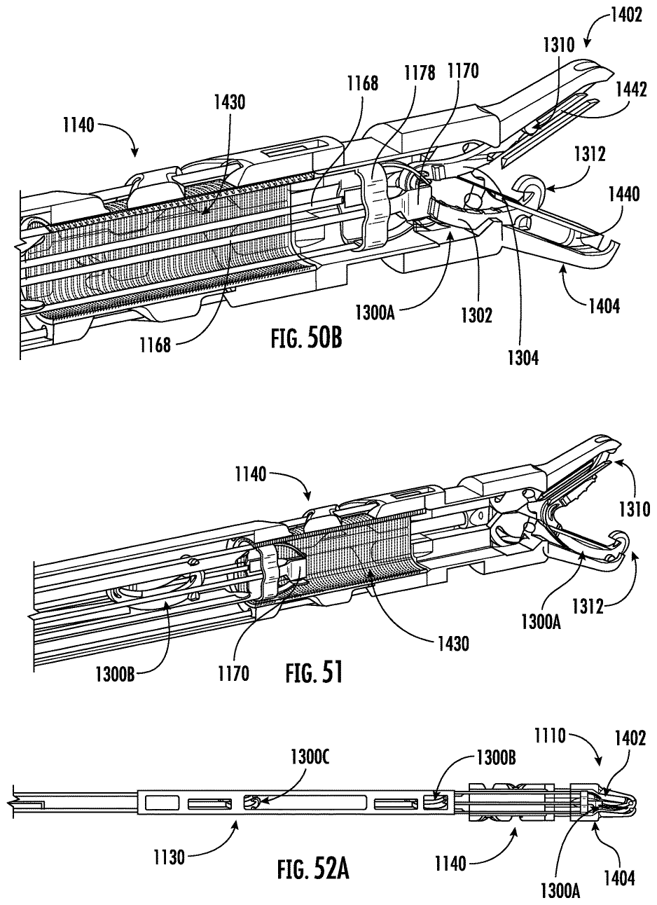

As shown in FIG. 35A, clip 1300 further includes a protrusion 1332 extending from either side of hook 1312 that facilitates guidance of clip 1300 through a guide track 1442 of jaw 1404 as clip 1300 is advanced into the jaws (see FIG. 50B). In one embodiment, bosses 1320, 1322 on latch 1310 are configured to also function as a guide protrusion that advances through guide track 1440 of jaw 1402. The boss's function to engage tracks 1440, 1442 of jaws 1402, 1404 if drive member 1130 becomes disengaged with clip 1130 during advancement, thereby preventing premature disengagement of the clip 1300 from jaws 1111, 1112.

In certain embodiments, arms 1302, 1304 of clip 1300 each include one or more protrusions 1326 extending from a proximal portion of the arms (distal of hinge 1306). In one embodiment, a protrusion 1326 extends on both sides of each of the arms 1302, 1304. Protrusions 1326 are designed to engage advancer tabs 1182 of drive member 1130. In particular, advancer tabs 1182 are designed to snap inwardly against clip 1300 just proximal of protrusions 1326. Since tabs 1182 are biased inwardly, distal movement of drive member 1130 will cause tabs to contact and engage a proximal side of protrusions 1326, thereby allowing the drive member 1130 to advance the clips within cartridge 1120 (see FIG. 26C).

FIGS. 36A and 36B illustrate drive member 1130 coupling with a clip 1300. As shown, retainer tabs 1170, 1172, 1174, 1176 are biased inwardly such that they clamp onto a proximal end portion of clip 1300 (around hinge 1306). This allows drive member 1130 to retain and control clip 1300 as it advances distally through wrist assembly 1140 into end effector 1110. In addition, it allows drive member 1130 to retain control of clip 1300 as arms 1302, 1304 of clip 1300 are opened within jaws, 1111, 1112.

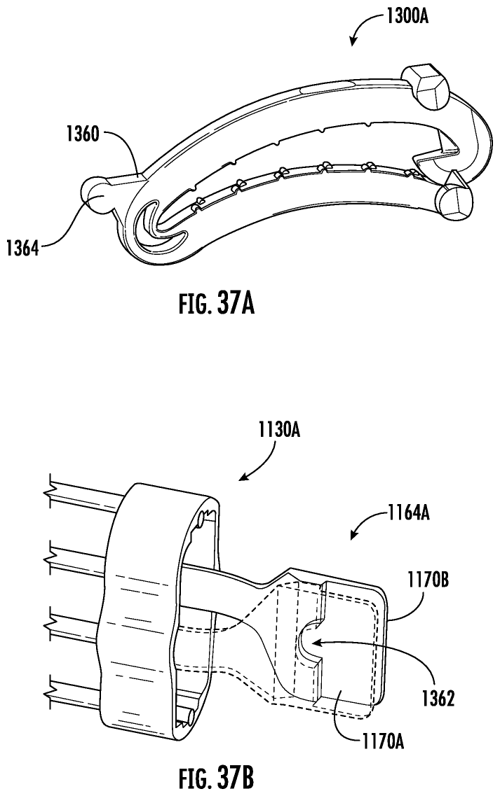

FIGS. 37A and 37B illustrate an alternative embodiment of clip 1300A and distal component 1164A of drive member 1130A. As shown, clip 1300A includes a proximal handle 1360 to facilitate engagement with retainer tabs 1170A, 1712A of drive member 1130A. Drive member 1130A may further include an internal recess 1362 disposed between tabs 1170A, 1170B that is designed to engage with, and removably couple to, a distal protrusion 1364 on handle 1360. This design provides a secure coupling between distal component 1164A of drive member 1130A and clip 1300A.

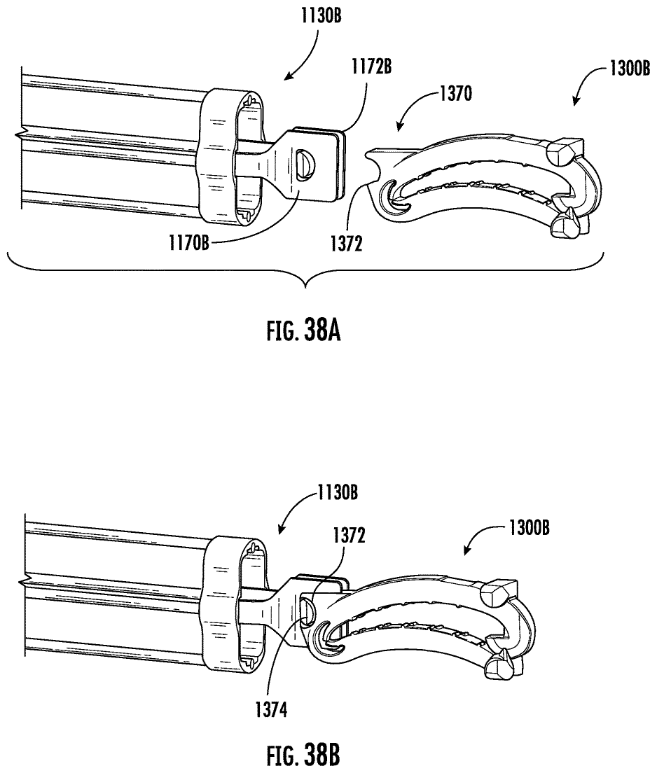

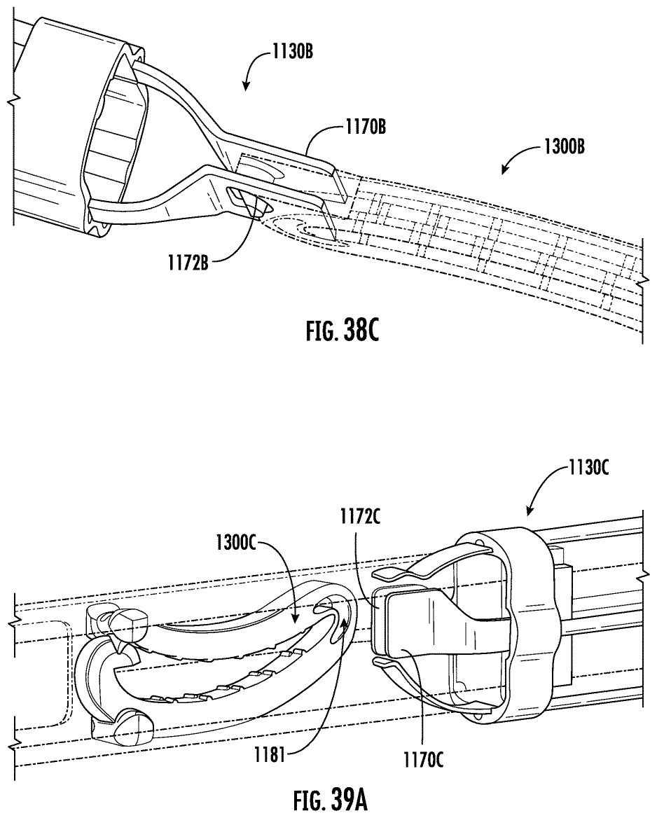

FIGS. 38A-38C illustrate yet another embodiment of a drive member 1130B and a clip 1300B. As shown, clip 1300B includes a proximal handle 1370 with an engagement feature 1372 that is designed to removably couple to an internal recess or engagement feature 1374 disposed within retainer tabs 1170B, 1172B of drive member 1130B.

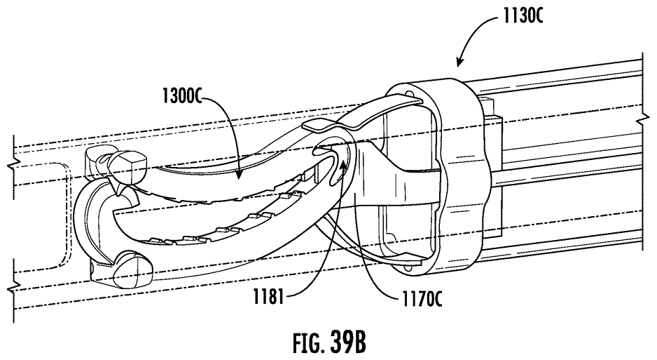

FIGS. 39A and 39B illustrate yet another embodiment of a drive member 1130C and a clip 1300C. In this embodiment, clip 1300C includes a proximal opening 1181 sized to receive retainer tabs 1170C, 1172C of drive member 1130C. Retainer tabs 1170C, 1172C may be biased outwardly from the longitudinal axis in this embodiment. Thus, retainer tabs 1170C, 1172C are moved distally into proximal opening 1181 and then biased outward to secure tabs 1170C, 1172C within opening 1181.

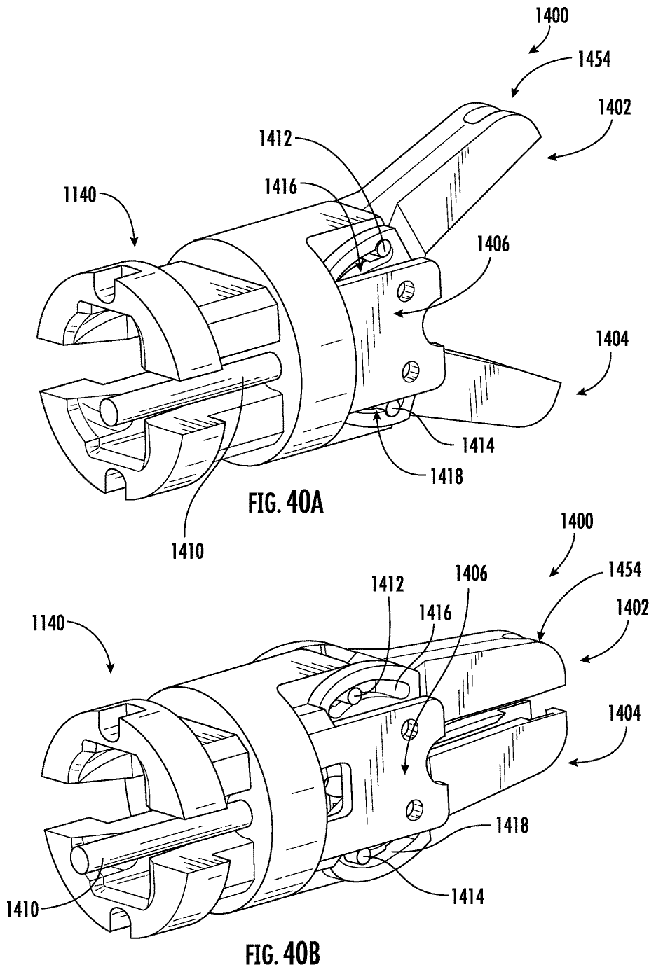

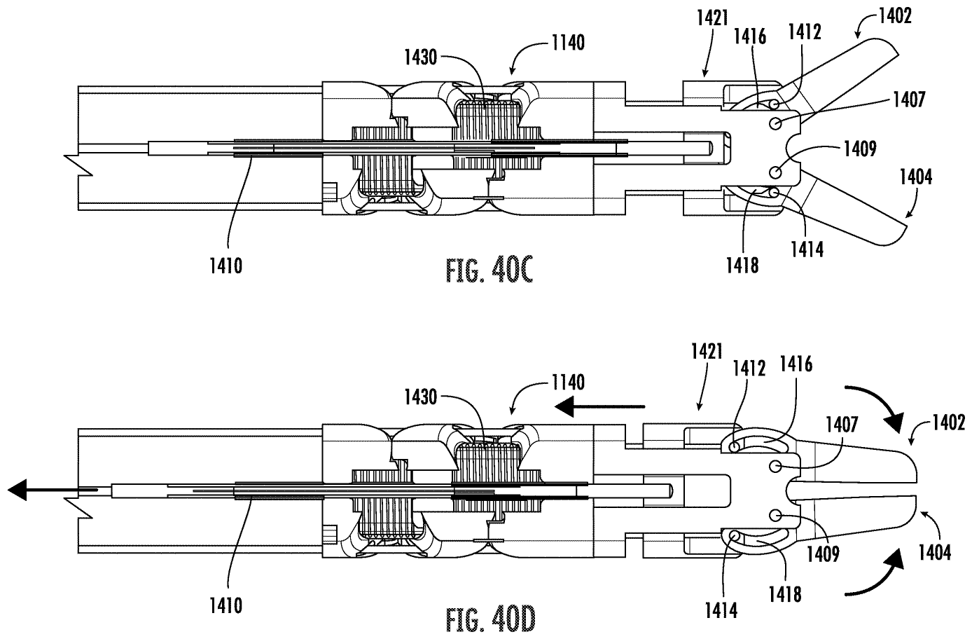

Referring now to FIGS. 40A-40D, one embodiment of a jaw assembly 1400 will now be described. Jaw assembly 1400 may be used with instrument 100, instrument 1100 or any other suitable clip applier instrument. As shown, jaw assembly 1400 comprises first and second jaws 1402, 1404 that are pivotally coupled to each other at first and second pivot pins 1407, 1409. First and second jaws 1402, 1404 are preferably designed to move relative to each other between an open position (as shown in FIG. 40A) and a closed position, wherein the distal ends of the jaws are substantially parallel with each other (see FIG. 40B). In the preferred embodiment, both jaws 1402, 1404 are movable jaws, although it will be recognized that one of the jaws may be a movable jaw configured to move between open and closed positions relative to the other jaw.

Instrument 1100 comprises an actuator rod or cable drive 1410 extending through shaft 1105 and wrist assembly 1140 into jaws 1402, 1404 for opening and closing the jaws. Cable drive 1410 preferably extends laterally outside of cartridge 1120, drive member 1130 and an internal tube 1430 passing through wrist assembly 1140 (see FIG. 42A discussed further below). Longitudinal translation of cable drive 1410 (i.e., pushing/pulling) causes the jaws 1402, 1404 to open and close. In certain embodiments, jaws 1402, 1404 may be opened by distal movement of cable drive 1410 (and closed by proximal movement of cable drive 1410). In other embodiments, jaws 1402, 1404 may be closed by distal movement of cable drive 1410 (and opened by proximal movement of cable drive 1410).

Referring now to FIGS. 40C and 40D, cable drive 1410 is coupled to a support member 1421 that includes first and second slot pins 1412, 1414 extending laterally outward from support member 1421. Slots pins 1412, 1414 are configured to slide within first and second curved slots 1416, 1418 in first and second jaws 1402, 1404, respectively. Slot pins 1412, 1414 are configured to slide distally and proximally with the similar movements of cable drive 1410 such that, for example, distal advancement of cable drive 1410 causes slot pins 1412, 1414 to slide to the distal end of slots 1416, 1418, thereby causing the jaws to pivot around about pivot pins 1407, 1409 into the open position (see FIG. 40C). Likewise, proximal withdrawal of cable drive 1410 causes slot pins 1412, 1414 to slide to the proximal ends of slots 1416, 1418, thereby pivoting jaws around pivot pins 1407, 1409 the closed position (see FIG. 40D).

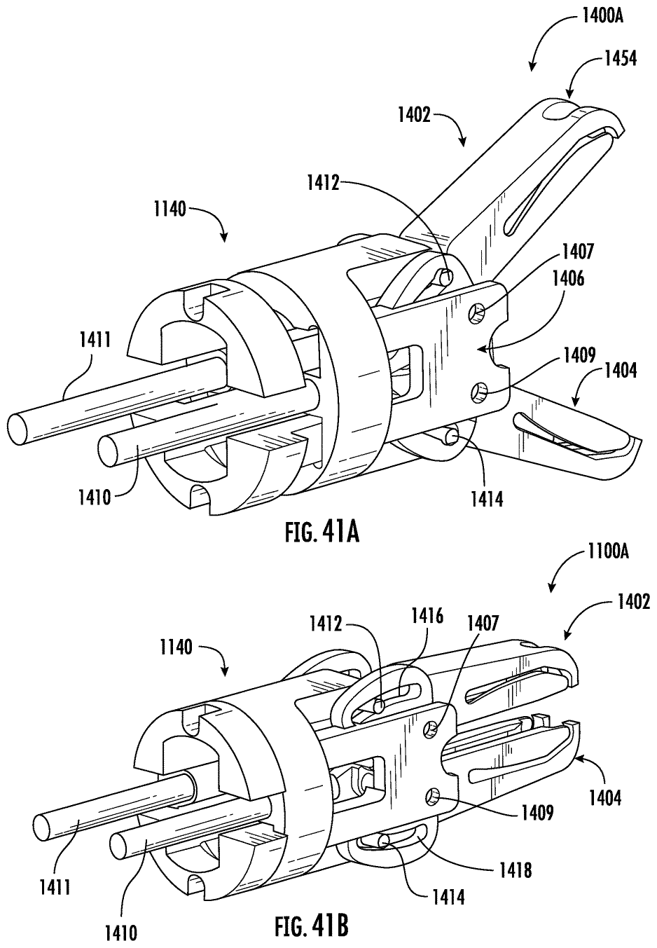

FIGS. 41A and 41B illustrate an alternative embodiment of jaw assembly 1400A. Jaw assembly 1400A is similar in most respects to assembly 1400 except that it includes first and second actuator rods or cable drives 1410, 1411 extending laterally outside of cartridge 1120, drive member 1130 and an internal tube 1430 passing through wrist assembly 1140 (see FIG. 42B discussed further below). Cable drive 1411 operates in the same manner as drive 1410. Longitudinal translation of cable drive 1411 together with drive 1410 causes slot pins to slide through slots in upper and lower jaws 1402, 1404, causing the jaws to pivot between the open and closed positions.

In certain embodiments, the slots are substantially linear. In other embodiments, the slots may be non-linear and/or curved. For example, a non-linear slot may have a curvature from the proximal end to the distal end. The non-linear slot may be shaped such that a grip force applied by at least one of the first and second jaws is substantially proportional to a force applied to the pin as the pin is translated from the proximal end to the distal end of the non-linear slot. In certain embodiments, the non-linear slot is shaped such that the first and second jaws apply a substantially constant grip force therebetween as the pin is translated from the proximal end to the distal end of the slot. This provides a constant mechanical advantage between the force applied to the pin and the force applied by the jaws to tissue held therebetween, thereby allowing a user (or a robotic system) to more easily regulate the forces applied to tissue by the jaws. In addition, this design allows for a substantially constant grip force to be applied by the jaws regardless of the angle between the jaws. A more completed description of a non-linear slot can be found in commonly assigned, U.S. patent application Ser. No. 17/081,088, the complete disclosure of which is incorporated herein by reference.

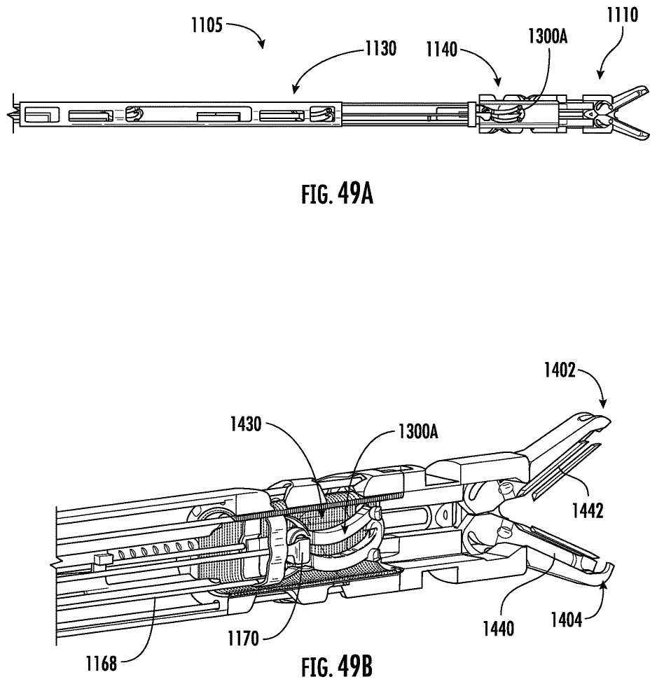

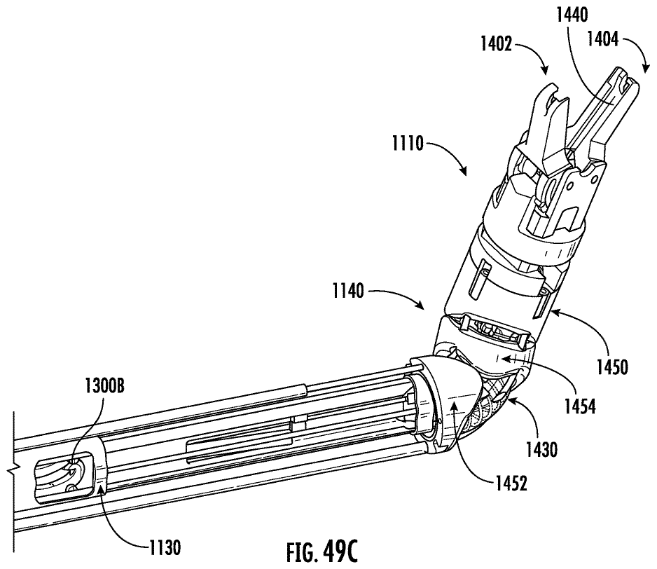

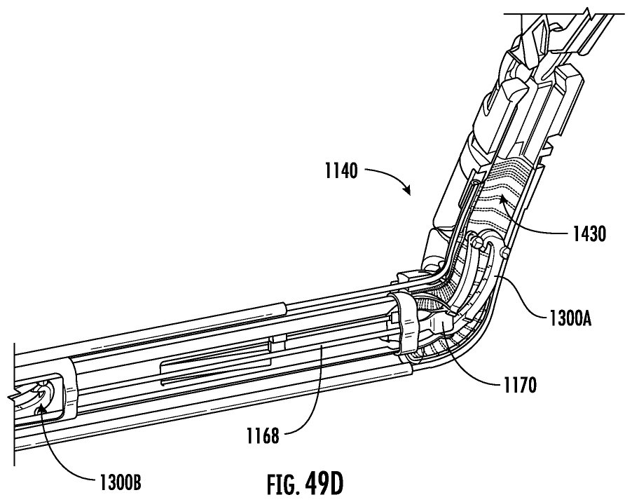

As shown in FIGS. 49B and 50B, first and second jaws 1402, 1404 each include guide tracks 1440, 1442 that generally extend from a proximal portion of the jaws to the distal end 1434, 1436 of each jaw. Guide tracks 1440, 1442 are configured to receive bosses 1320, 1322 and 1324 of clip 1300, which slide along guide tracks 1440, 1442 as the clip 1300 is delivered distally by drive member 1130. This ensures that each arm 1302, 1304 of clip 1300 is appropriately delivered to each jaw 1402, 1404 (discussed in more detail below).

Jaws 1402, 1404 may each include an engagement feature at their distal ends to secure the clips therein after they have been delivered by drive member 1130. The engagement features allow drive member 1130 to be released from the clip after the clip has been secured to jaws 1402, 1404. Thus, the force required to disengage retainer tabs 1170, 1172, 1174, 1716 from the clip is less than the force required to disengage the clip from the engagement features. In addition, these engagement features ensure that the clip does not fall out of the jaws 1402, 1404 before they have been closed and latched onto tissue or a vessel at the target site.

In one embodiment, these engagement features comprise ramped leaf springs (not shown) located in, or near, guide tracks 1440, 1442. These leaf springs are similar in design to leaf springs 439 discussed in reference to jaw assembly 400 and shown in FIG. 10E above. Guide tracks 1440, 1442 taper inwardly in the distal direction such that the lateral width across guide tracks 1440, 1442 decreases distally. As the clip is advanced distally through guide tracks 1440, 1442, they contact the outer surfaces of guide tracks 1440, 1442, which become narrower as the clip is advanced distally. The clip presses against the leaf springs so that they are biased outwardly to allow the clip to move to the distal ends of the jaws. This spring pressure applied inwardly by the leaf springs retains the clip within the distal ends of the jaws and inhibits them from withdrawing proximally and/or falling out of the jaws.

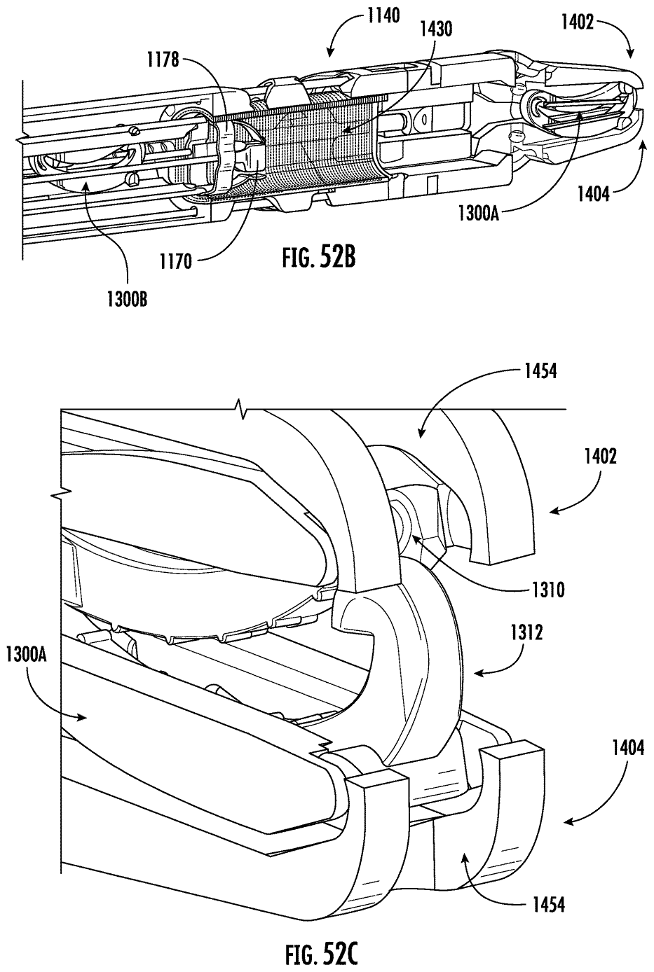

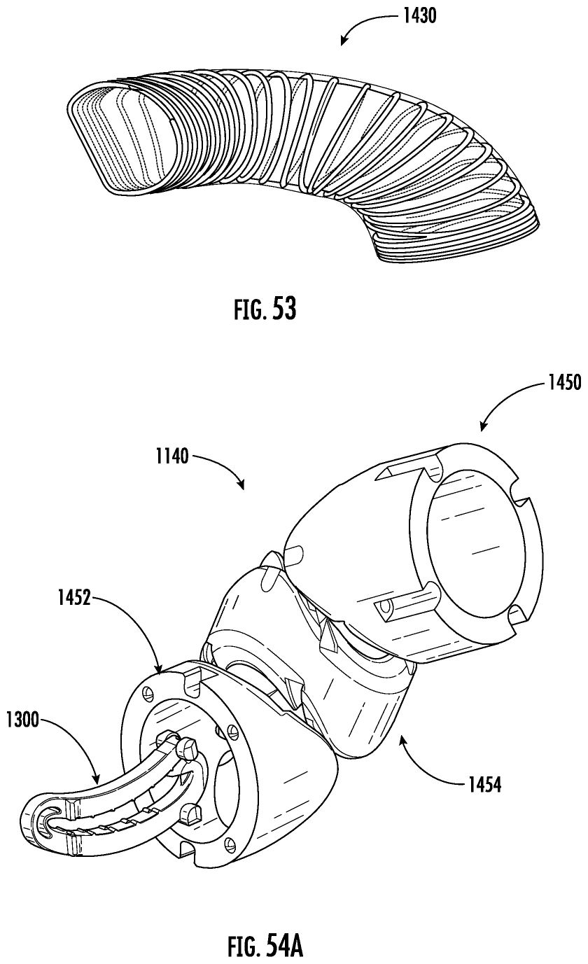

In addition, jaws 1402, 1404 each have distal end portions 1434, 1436 that include a cutout 1454 (see FIG. 52C). Cutouts 1454 are disposed at the distal end of guide tracks 1440, 1442. Cutouts 1454 preferably have a larger cross-sectional area than guide tracks 1440, 1442 and function to accommodate hook 1312 and latch 1310 of clip 1300. This ensures that the jaws can be opened and removed from clip 1300 after the clip has been closed and latched onto tissue or a vessel.

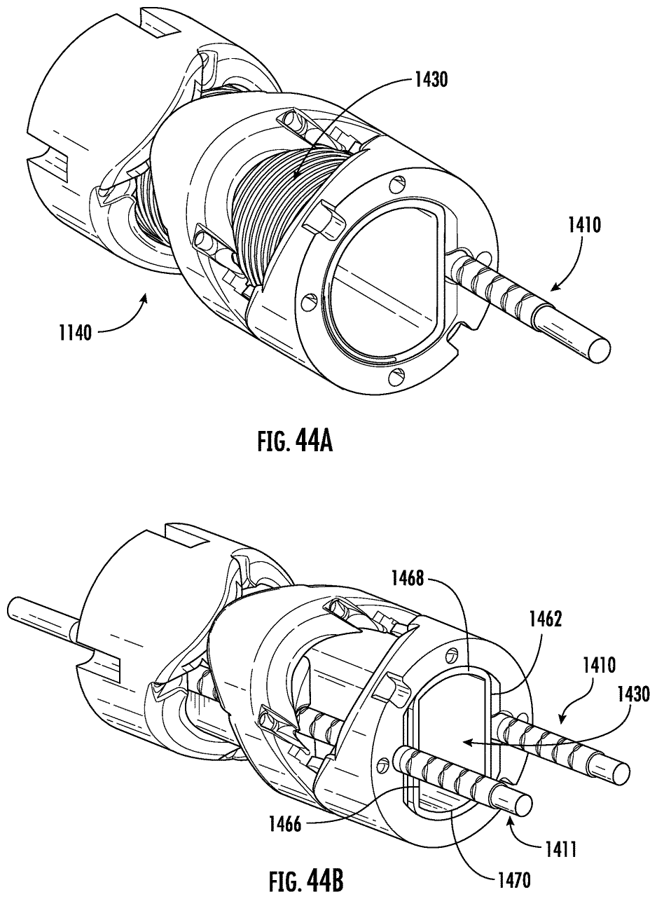

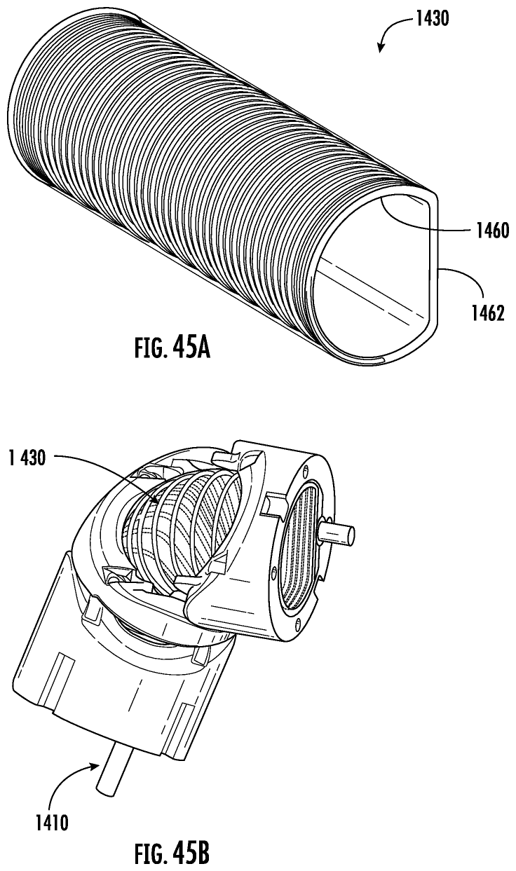

Referring now to FIGS. 42, 43A and 243B, a wrist assembly 1140 is now described. Wrist assembly 1140 may be used within instrument 100, instrument 1100 or any other suitable clip applier instrument. Wrist assembly 1140 comprises multiple linkages or discs that allow for articulation of end effector 1110 and shaft 1105 in at least two axes perpendicular to the longitudinal axis of shaft 1105 (i.e., the “yaw” and “pitch” axes). As shown, wrist assembly 1140 includes a distal linkage or disc 1450 coupled to jaws 1111, 1112, a proximal linkage or disc 1452 coupled to shaft 1105 and a middle linkage or disc 1454 therebetween. In one embodiment, middle disc 1454 is rotatably coupled to proximal disc 1452 to allow for rotation about one of the axes (see FIG. 41B) and rotatably coupled to distal disc 1450 for rotation about another of the axes (see FIG. 44A).

In a preferred embodiment, distal disc 1450 is fixed to end effector 1110 and proximal disc 1452 is fixed to shaft 1105. Thus, the rotation or articulation only occurs between the middle disc 1454 and the proximal and distal discs 1450, 4152. This configuration “decouples” the end effector 1110 and jaws 1111, 1112 from the wrist assembly 1140 such that the end effector 1110 itself does not articulate, which provides more control and precision for the surgeon in positioning the jaws 1111, 1112 in a proper orientation for applying a clip to tissue or a vessel.

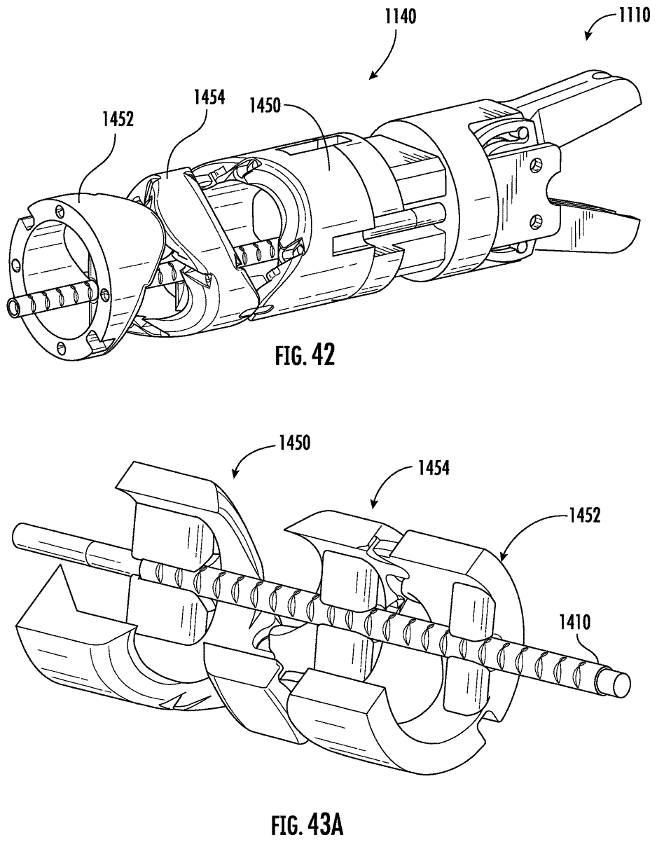

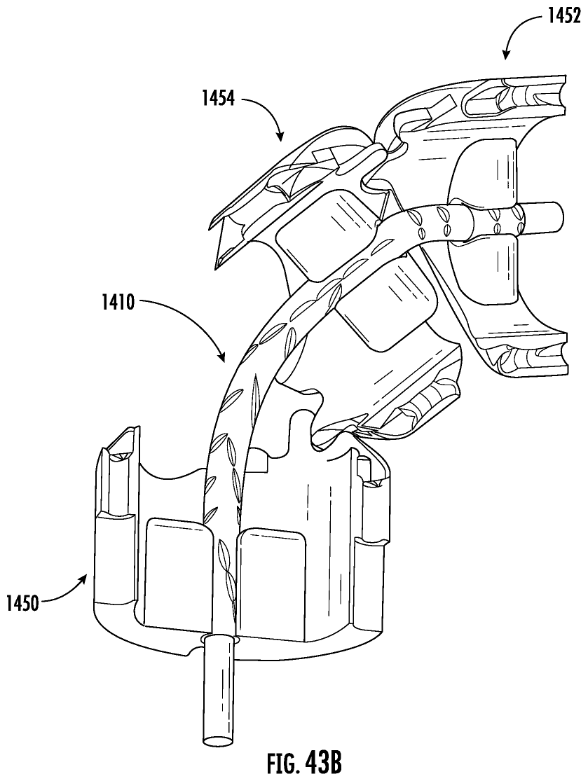

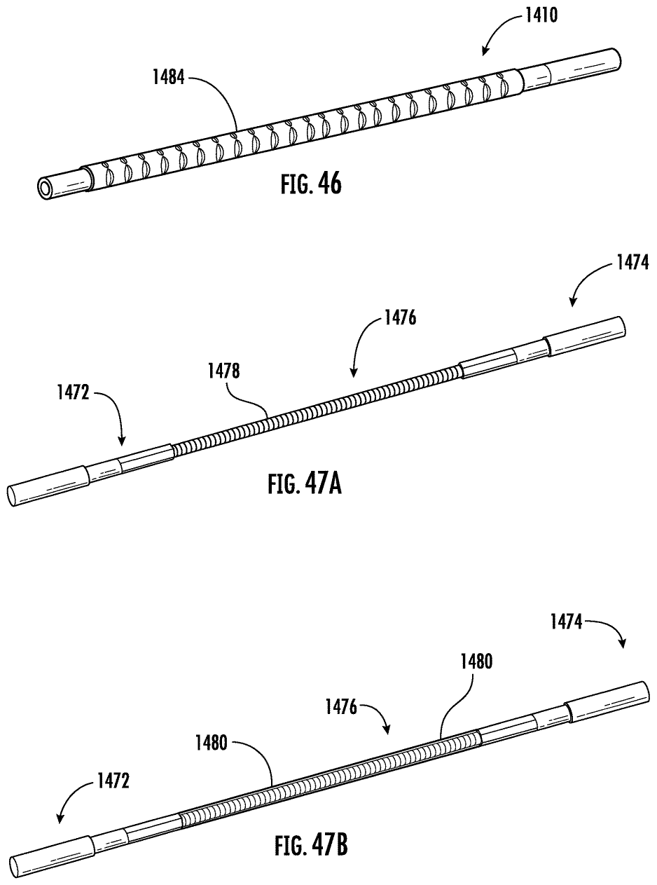

Actuator rod/cable 1410 (and rod 1411 in certain embodiments) extends through wrist assembly 1140, preferably through a flexible sheath 1484 (see FIG. 46) that is anchored to the distal disc 1450 and slidingly coupled through internal cutouts in each of the remaining discs 1452 and 1454 (see FIG. 43A). The flexible sheath 1484 guides and supports the actuator cable/rod 1410 to deliver a pushing force through the articulated wrist and up to the jaws without buckling. Suitable materials for sheath 1484 include, but are not limited to, laser cut stainless steel tubing or polymer tubing materials. The sheath 1484 must be flexible enough to follow the curvature of an articulated wrist while still radially rigid enough to sufficiently contain the actuator cable/rod and its pushing/pulling forces. In certain embodiments, actuator rod 1410 is secured, or anchored, to support member 1421 and slidingly coupled through the flexible sheath 1484, which is slidingly coupled through middle disc 1454 and proximal disc 1452. This allows the proximal end of rod 1410 and the proximal end of the sheath 1484 to pay in and out of wrist assembly 1140 as the wrist assembly 1140 articulates. As shown in FIG. 43B, as wrist assembly 1140 articulates, the length of at least a portion of rod 1410 and sheath 1484 must increase because the distance between shaft 1105 and end effector 1110 increases (discussed in more detail below). Providing a sliding fit between rod 1410, the sheath 1484 and the middle and proximal discs 1454, 1452 allows a portion of rod 1410 and sheath 1484 to increase in length with wrist assembly 1140 to accommodate for this articulation.

Referring now to FIGS. 44A and 44B, internal tube 1430 extends from shaft 1105 to end effector 1110 and provides a flexible, smooth channel for passage of drive member 1130 and clips 1300 therethrough even when wrist assembly 140 is articulating such that end effector 1110 and shaft 1105 are not oriented in a parallel direction (see FIG. 43B). In certain embodiments, tube 1430 comprises an embedded coil surrounded by an elastic polymer jacket and is bonded to the coil. This provides an overall flexible structure that inhibits kinking during tight bends of wrist assembly 1140. Suitable materials for the polymer jacket include, but are not limited to, durable and highly elastic polymers, such as Pebax shore 35D, Tecoflex shore 80A and Pellethane shore 80A. Suitable materials for the embedded coil include, but are not limited to, 0.005 to 0.010 diameter stainless steel or nitinol.

Internal tube 1430 is preferably constructed with a cross-section that accommodates actuator rod(s) 1410 and/or 1411. In one embodiment, tube 1430 includes a cross-section with a semi-circular portion 1460 and a substantially linear portion 1462 that provides a substantially D-shaped cross-section (see FIG. 45A). This cross-section allows for actuator rod 1410 to extend alongside linear portion 1462 laterally outward from tube 1430, thereby providing space within wrist assembly 1140 to allow for passage of drive member 1130 and clips 1300 therethrough.

In another embodiment, tube 1430 includes a cross-section with first and second substantially linear portions 1462, 1466 and first and second semi-circular portions 1468, 1470 extending between linear portions 1462, 1466 (see FIG. 44B). This cross-section allows for both actuator rods 1410, 1411 to extend alongside linear portions 1462, 1466.

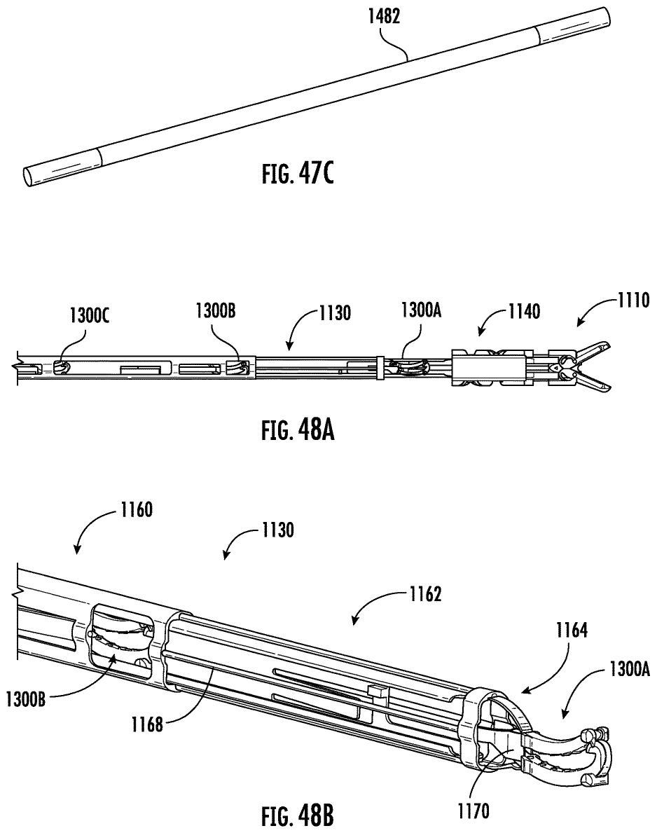

Referring now to FIGS. 46 and 47A-47C, an embodiment of actuator rods or cable drives 1410, 1411 will now be described. Actuator rod(s) 1410, 1411 may be used with instrument 100, instrument 1100 or any other suitable clip applier instrument. As shown in FIG. 47A, rod 1410 includes a proximal component 1472, a distal component 1474 and a middle, flexible component 1476. Flexible component 1476 is designed to bend or articulate within wrist assembly 1140. At least a portion of flexible component 1476 may also be designed to expand or contract in the longitudinal direction to accommodate increased or decreased distances between shaft 1105 and end effector 1110 as wrist assembly 1140 articulates.

In one embodiment, flexible component 1476 comprises a braided tungsten cable 1478 and rigid components 1472, 1474 comprise a stainless steel pin or tube. The braided tungsten cable 1478 may be secured to the stainless steel pin or tube by any suitable method, such as crimping, welding or the like.

As shown in FIG. 47B, flexible component 1476 may include a flexible PTFE heat shrink tubing 1480 surrounding cable 1478 to contain the cable strands when rod 1410 is, for example, under compression during pushing of the rod 1410. Rod 1410 may include a second heat shrink tubing 1482 (see FIG. 47C) overlying tubing 1480 and extending over some portion of rigid components 1472, 1474 to provide a continuous grip cable outer diameter.

As shown in FIG. 46, a flexible sheath 1484 is provided over the second heat shrink tubing 1482 to provide a sliding fit between the underlying cable 1478 and sheath 4182. This allows rod 1410 to flex and bend, and/or to contract and expand in length within the wrist assembly 1140. This also prevents grip cable buckling when rod 1410 is pushed. Suitable materials for sheath 1484 include, but are not limited to, laser cut stainless steel tubing or polymer tubing materials.

Referring now to FIGS. 26A-26C and 49A-53B, a method for applying multiple clips to tissue or vessels in a patient will now be described. As shown in FIG. 26B, drive member 1130 is first withdrawn proximally such that retainer tabs 1174, 1176 flex outward and ride over upper and lower surfaces 1136, 1138 of the clip cartridge housing 1134. This allows retainer tabs 1170, 1172 to spring inwardly to grasp and secure to clip 1300. At the same time, advancer tabs 1182 spring inwardly to contact and engage a proximal surface of the proximal clips (see FIG. 26C).

Drive member 1130 is then advanced distally until distal component 1164 advances past the distal end of cartridge housing 1134. As this occurs, the distal most clip 1300A is advanced forward with retainer tabs 1170, 1172 and the proximal clips 1300B, 1300C, etc. are moved forward with advancer tabs 1182. Once distal component 1164 moves distally of cartridge housing 1134, retainer tabs 1174, 1176 spring downwards and upwards to secure to the upper and lower surfaces of clip 1300A (see FIG. 48B).

As shown in FIG. 49B, drive member 1130 is then advanced distally to advance the distal clip 1300A into end effector 1110. Drive member 1130 and clip 1300A pass through internal tube 1430 as they pass through wrist assembly 1140. As discussed previously, internal tube 1430 can bend and flex and provide a smooth conduit for drive member 1130 and clip 1300A even when end effector 1110 is articulated relative to shaft 1105 (see FIG. 49C and D).

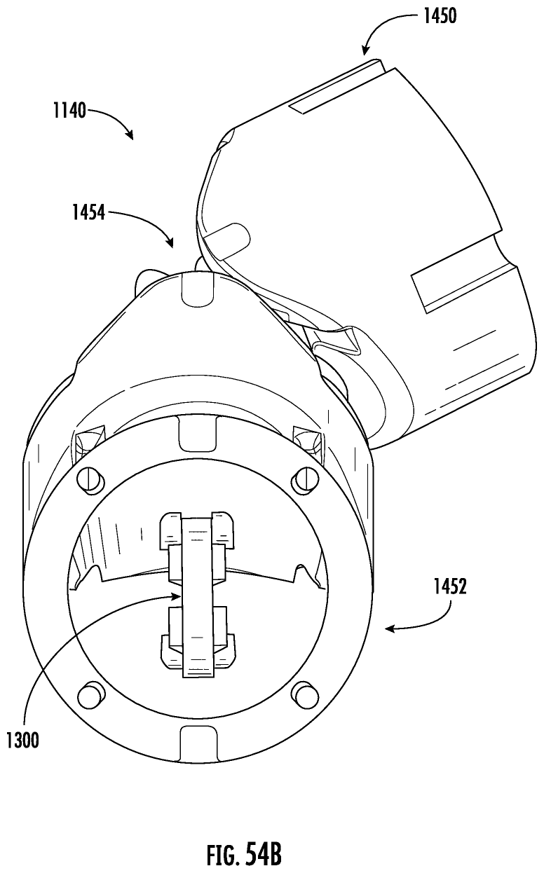

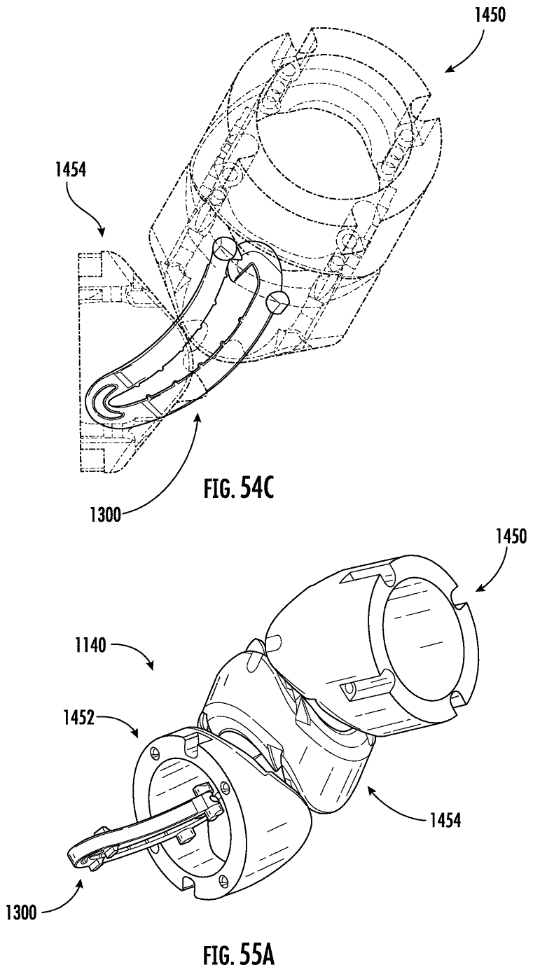

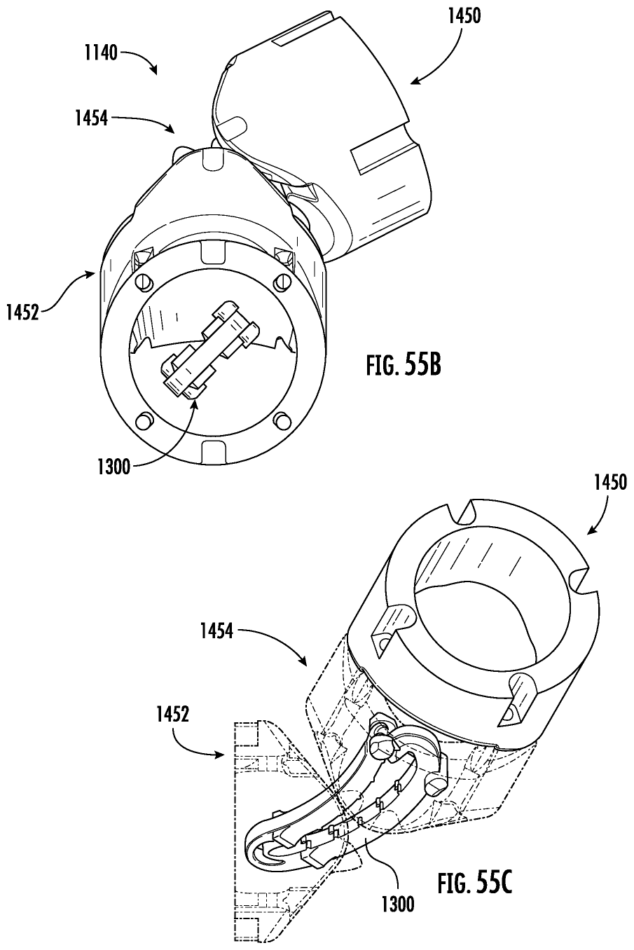

In a preferred embodiment, clip 1300A is oriented at about a 30 to 60 degree angle, preferably about a 45 degree angle, relative to a plane passing through shaft 1105 or the wrist axis (see FIGS. 55A-55C). This angle provides less contact between clip 1300A and the inner surface of tube 1430 when wrist assembly 1140 is articulated than if, for example, clip 1300A were oriented at an orthogonal angle to the wrist axis (see, for example, FIGS. 54A-54C). Reducing the amount of contact between the clip and tube 1430 reduces the amount of force requiring to push the clip through the wrist assembly and into the jaws.

As distal clip 1300A is moved into the jaws 1402, 1404, advancer tabs 1182 are advancing the more proximal clips (1300B, 1300C, etc.) distally to the next distal position within clip cartridge 1120. These clips will then be in position for engagement with retainer tabs 1170, 1172 after the distal clip 1300A has been released and drive member 1130 has been withdrawn back into its original position (see FIG. 26B).

Referring now to FIGS. 50A and 50B, drive member 1130 advances clip 1300 into jaws 1402, 1404 such that bosses 1320, 1322 and 1324 of latch 1310 and hook 1312 slide through guide tracks 1440, 1442. This ensures that arms 1302, 1304 of clip 1300 open up and advance to the distal end of jaws 1402, 1404, thereby placing clip 1300 in position to be closed and latched by jaws 1402, 1404 (see FIGS. 52A-52C). As arms 1302, 1304 of clip 1300 slide through guide tracks 1440, 1442, they engage the ramped leaf springs at the distal ends of the jaws 1402, 1404. These leaf springs secure arms 1302, 1304 of clip 1300 to the jaws.

After the clip has been delivered to the jaws, drive member 1130 may be released from clip 1300 and withdrawn proximally back into shaft 1105 to retrieve another clip 1300B (see FIGS. 51 and 352B). In some embodiments, drive member 1130 is withdrawn when jaws are open. In other embodiments, drive member 1130 may be withdrawn after jaws are closed.

The surgical instruments described herein may be coupled to a proximal control system that monitors and controls the linkages or discs in wrist assembly for articulating end effector 110 and the jaws relative to shaft 105 and for translating drive member 130 distally and proximally to deliver clips to the jaws. In addition, the control system may monitor and control the longitudinal location of drive member 130 relative to each of the clips within cartridge 120. In particular, the control system may monitor the location of the distal engagement elements of the drive member along cartridge 120 to determine when the drive member should be translated distally or proximally.

For example, the control system may monitor and control drive member such that these engagement elements are translated proximally until they are located over the openings in the upper and lower cartridge housing associated with the first distal-most clip in the cartridge. The control system may then monitor and control drive member 130 such that the engagement elements are translated distally until the distal-most clip is located in the desired location within jaws 402, 404. The control system may monitor and control proximal withdrawal of the drive member 130 after the clip has been latched and secured to tissue and/or a vessel to prevent in advertent disengagement of the drive member and the clip prior to that occurrence. The control system may also monitor and control movement of the drive member to a location on the cartridge associated with the most distal clip remaining in the cartridge.

This control system may be a manual control system with user interfaces that allow the user to control each of the functions of the instrument, or it may be an automatic control system that monitors and controls these functions. In some embodiments, the control system is a combination of manual and automatic that allows the user to adjust or control certain functions, while automatically limiting those functions within certain ranges or parameters.

In certain embodiments, the instrument may include sensors (not shown) for detecting a location of the engagement elements. The sensors may include any suitable sensors for detecting location, force and/or torque. In one embodiment, the sensors include fiber optic bend sensors, such as Fiber Bragg Gratings (FBG) for providing strain measurements in the jaws, the tension bands and/or other components of the surgical instrument. Various systems and methods for monitoring the shape and relative position of an optical fiber in three dimensions are described in U.S. patent application publication no. 2006/0013523, filed on Jul. 13, 2005, and U.S. Pat. No. 6,389,187, filed on Jun. 17, 1998, the completed disclosures of which are incorporated herein by reference for all purposes.

The control system may include one or more processors (e.g., microprocessor, microchip, or application-specific integrated circuit), one or more memory devices (e.g., random-access memory and/or read-only memory), and I/O interface and/or a communication interface. The processors may include one or more computer-readable storage devices and/or software applications that store program instructions that allow the processor(s) to compare the detected torque or force with the prescribed range. The I/O devices can include one or more devices that enable the user to interact with the system (e.g., a user interface). The I/O devices can include, for example, a touchscreen display, a keypad, one or more selectors, one or more indicators.

Although described as a processor, it is to be appreciated that the controllers may be implemented in practice by any combination of hardware, software, and firmware. Also, their functions as described herein may be performed by one unit, or divided up among different components, each of which may be implemented in turn by any combination of hardware, software, and firmware.

With reference to FIG. 56, an exemplary embodiment of a teleoperated surgical instrument 500 that may support a previously described instrument is depicted. As shown, the instrument 500 generally includes a proximal housing 510 at its proximal end and coupled to shaft 520 of the instrument. Proximal housing 510 may include an instrument memory or storage device (not shown). The memory can perform a number of functions when the instrument is loaded on a manipulator arm (not shown) of a robotic control system. For example, the memory can provide a signal verifying that the instrument is compatible with that particular surgical system. Additionally, the memory may identify the instrument and end effector type (whether it is a scalpel, a needle grasper, jaws, scissors, a clip applier, an electrocautery blade, or the like) to the surgical system so that the system can reconfigure its programming to take full advantage of the instrument's specialized capabilities. As further discussed below, the memory may include specifics on the architecture of the instrument, and include particular values that should be employed in control algorithms, such as tool compliance and gain values.

Proximal housing 510 also may include a force/torque drive transmission mechanism (not shown) for receiving output from the motors of the manipulator arm. The force/torque drive transmission mechanism transmits the output from the motors to an end effector 530 of the instrument through an instrument shaft 520 mounted to the transmission mechanism. Exemplary surgical robotic instruments, instrument/manipulator arm interface structures, and data transfer between the instruments and servomechanism is more fully described in U.S. Pat. No. 6,331,181, the full disclosure of which is incorporated herein by reference.

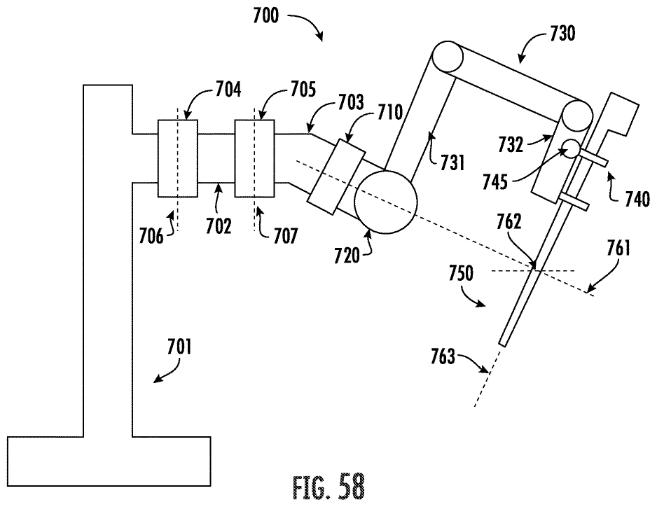

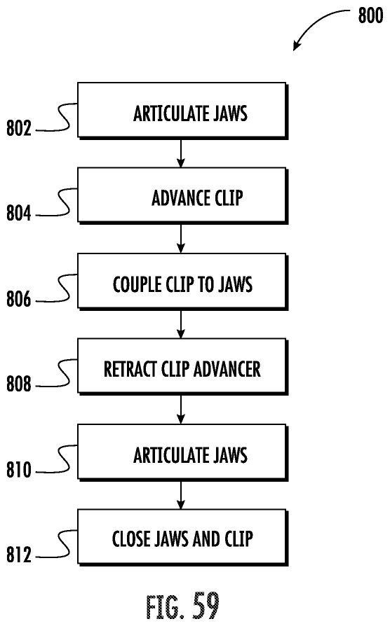

FIG. 59 illustrates a flow chart of a process 800 that may be carried out by a control system, such as a robotic control system (such as the one shown in FIGS. 57 and 58 and described below) that is coupled to a proximal housing or backend mechanism 510 of the surgical instrument. The robotic control system includes at least one processor that relays input commands from master controllers operated by the user to the first and second actuation systems within backend mechanism. The actuation systems then provide mechanical actuation and control of the instrument to perform various functions, such as articulation and clip application in response to manipulation of the master input devices In one embodiment, the backend mechanism includes a first drive system for controlling articulation of the end effector relative to the shaft and a second drive system for controlling longitudinal translation of the drive member through the shaft to advance clips into the jaws and retract the drive member after the clips have been coupled to the jaws and/or closed and sealed onto a vessel. The backend mechanism may include a third drive system for opening and closing the jaws and/or a fourth drive or control system for monitoring and controlling the longitudinal location of the drive member (i.e., the clip advancer) within the instrument shaft.

In one embodiment of process 800, the control system may be operated to actuate the first drive system in the backend mechanism 510 to articulate the end effector, e.g., to straighten a bent wrist such that the end effector is substantially parallel to the shaft (see step 802). Once the wrist has been straightened, the control system may be operated to actuate the second drive system in the backend mechanism to advance the drive member distally to advance a first or distal-most clip into the jaws of the instrument (see step 804). In some embodiments, the jaws are opened prior to advancing the clip into the jaws. In other embodiments, the jaws may be closed or partially open. Once the clip has been coupled to the jaws (step 806), the control system may be operated to actuate the second drive system to retract the drive member proximally from the jaws so that it aligns with a second (or the next distal-most) clip in the clip cartridge (see step 808). In some embodiments, the clips will be advanced together such that the second clip is advanced to the position previously occupied by the first clip as the first clip is advanced into the jaws. In these embodiments, the drive member will be retracted to the same position relative to the instrument or clip cartridge to engage the second clip as the first clip. In other embodiments, the drive member may be retracted more proximally to engage the second clip (if the second clip was not advanced distally in the same operation as the first clip). In these embodiments, the control system may include sensors, controllers, or other mechanisms for determining the location of the clip advancer to ensure that it is retracted to a position corresponding with the second clip in the cartridge (as discussed previously). It should be noted that any of the above-described drive systems may be independent of each other, or they may be combined with each other such that, for example, one drive system drives two functionalities, such as, for example rotation of the end effector and clamping of the jaws.

The control system may then be operated to actuate the first drive system to articulate the end effector to rotate the jaws relative to the shaft in order to, for example, position the jaws around a targeted vessel or tissue (step 810). The control system may then be operated to actuate the third drive system to close the jaws such that the clip is closed, latched, and sealed around the target vessel or tissue (step 812).

Of course, it will be recognized that that the drive member and clips may be advanced (and the drive member retracted) through the wrist to the end effector while the wrist is bent (i.e., while the end effector is rotated in the yaw, pitch or roll directions). As discussed above, the drive members described herein include flexible portions that bend or flex within the wrist of the instrument to allow the drive member to remain positioned in the wrist and the jaws during articulation of the end effector. Thus, in certain embodiments, the control system may be operated to first articulate the end effector such that the jaws are positioned around the target tissue or vessel and then advance the drive member and the first clip into the jaws.

As noted above, the present surgical instruments may be employed in a robotic teleoperated surgical system. FIG. 57 illustrates, as an example, a top view of an operating room employing a robotic surgical system. The robotic surgical system in this case is a robotic surgical system 600 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The servomechanism used for telesurgery will often accept input from two master controllers (one for each of the surgeon's hands) and may include two or more robotic arms. A surgical instrument is mounted on each of the robotic arms. Operative communication between master controllers and associated robotic arm and instrument assemblies is typically achieved through a control system. The control system typically includes at least one processor that relays input commands from the master controllers to the associated robotic arm and instrument assemblies and back in the case of, for example, force feedback or the like. One example of a robotic surgical system is the DA VINCI™M system commercialized by Intuitive Surgical, Inc. of Sunnyvale, California.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described in U.S. Pat. Nos. 7,594,912, 6,758,843, 6,246,200, and 5,800,423, the full disclosures of which are incorporated herein by reference in their entirety for all purposes. These linkages often manipulate an instrument holder to which an instrument having a shaft is mounted. Such a manipulator structure can include a parallelogram linkage portion that generates motion of the instrument holder that is limited to rotation about a pitch axis that intersects a remote center of manipulation located along the length of the instrument shaft. Such a manipulator structure can also include a yaw joint that generates motion of the instrument holder that is limited to rotation about a yaw axis that is perpendicular to the pitch axis and that also intersects the remote center of manipulation. By aligning the remote center of manipulation with the incision point to the internal surgical site (for example, with a trocar or cannula at an abdominal wall during laparoscopic surgery), an end effector of the surgical instrument can be positioned safely by moving the proximal end of the shaft using the manipulator linkage without imposing potentially hazardous forces against the abdominal wall. Alternative manipulator structures are described, for example, in U.S. Pat. Nos. 6,702,805, 6,676,669, 5,855,583, 5,808,665, 5,445,166, and 5,184,601, the full disclosures of which are incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. Manipulation and control of these end effectors is a particularly beneficial aspect of robotic surgical systems. For this reason, it is desirable to provide surgical tools that include mechanisms that provide two or three degrees of rotational movement of an end effector to mimic the natural action of a surgeon's wrist. Such mechanisms should be appropriately sized for use in a minimally invasive procedure and relatively simple in design to reduce possible points of failure. In addition, such mechanisms should provide an adequate range of motion to allow the end effector to be manipulated in a wide variety of positions.

The Console includes a monitor 604 for displaying an image of a surgical site to the Surgeon, left and right manipulatable control devices 608 and 609, a foot pedal 605, and a processor 602. The control devices 608 and 609 may include any one or more of a variety of input devices such as joysticks, gloves, trigger-guns, hand-operated controllers, or the like. The processor 602 may be a dedicated computer that may be integrated into the Console or positioned next to it.

The Surgeon performs a minimally invasive surgical procedure by manipulating the control devices 608 and 609 (also referred to herein as “master manipulators”) so that the processor 602 causes their respectively associated robotic arm assemblies, 628 and 629, (also referred to herein as “slave manipulators”) to manipulate their respective removably coupled surgical instruments 638 and 639 (also referred to herein as “tools”) accordingly, while the Surgeon views the surgical site in 3-D on the Console monitor 604 as it is captured by a stereoscopic endoscope 640.

Each of the tools 638 and 639, as well as the endoscope 640, may be inserted through a cannula or other tool guide (not shown) into the Patient so as to extend down to the surgical site through a corresponding minimally invasive incision such as incision 666. Each of the robotic arms is conventionally formed of links, such as link 662, which are coupled together and manipulated through motor controlled or active joints, such as joint 663.

The number of surgical tools used at one time and consequently, the number of robotic arms being used in the system 600 will generally depend on the diagnostic or surgical procedure and the space constraints within the operating room, among other factors. If it is necessary to change one or more of the tools being used during a procedure, the Assistant may remove the tool no longer being used from its robotic arm, and replace it with another tool 331 from a Tray (“T”) in the operating room.

The monitor 604 may be positioned near the Surgeon's hands so that it will display a projected image that is oriented so that the Surgeon feels that he or she is actually looking directly down onto the operating site. To that end, images of the tools 638 and 639 may appear to be located substantially where the Surgeon's hands are located.

The processor 602 performs various functions in the system 600. One function that it performs is to translate and transfer the mechanical motion of control devices 608 and 609 to their respective robotic arms 628 and 629 through control signals overbus 610 so that the Surgeon can effectively manipulate their respective tools 638 and 639. Another important function is to implement various control system processes as described herein.

Robotic surgery systems and methods are further described in U.S. Pat. No. 5,797,900, filed on May 16, 1997, issued on Aug. 25, 1998, U.S. Pat. No. 6,132,368, filed on Nov. 21, 1997, issued on Oct. 17, 2000, U.S. Pat. No. 6,331,181, filed on Oct. 15, 1999, issued on Dec. 18, 2001, U.S. Pat. No. 6,441,577, filed on Apr. 3, 2001, issued on Aug. 27, 2002, U.S. Pat. No. 6,902,560, filed on Jan. 6, 2004, issued on Jun. 7, 2005, U.S. Pat. No. 6,936,042, filed on Apr. 16, 2002, issued on Aug. 30, 2005, and U.S. Pat. No. 6,994,703, filed on Dec. 4, 2002, issued on Feb. 7, 2006, the full disclosures of which are incorporated herein by reference for all purposes. A suitable robotic surgical system currently in use is the da Vinci S Surgical System by Intuitive Surgical, Inc.

FIG. 58 illustrates, as an example, a side view of a simplified (not necessarily in proportion or complete) illustrative robotic arm assembly 700 (which is representative of robotic assemblies 628 and 629) holding a surgical instrument 750 (which is representative of tools 638 and 639) for performing a surgical procedure. The surgical instrument 750 is removably held in tool holder 740. The arm assembly 700 is mechanically supported by a base 701, which may be part of a patient-side movable cart o affixed to the operating table or ceiling. It includes links 702 and 703 which are coupled together and to the base 701 through setup joints 704 and 705.

The setup joints 704 and 705 in this example are passive joints that allow manual positioning of the arm 700 when their brakes are released. For example, setup joint 704 allows link 702 to be manually rotated r about axis 706, and setup joint 705 allows link 703 to be manually rotated about axis 707.

Although only two links and two setup joints are shown in this example, more or less of each may be used as appropriate in this and other robotic arm assemblies described herein. For example, although setup joints 704 and 705 are useful for horizontal positioning of the arm 700, additional setup joints may be included and useful for limited vertical and angular positioning of the arm 700. For major vertical positioning of the arm 700, however, the arm 700 may also be slidably moved along the vertical axis of the base 701 and locked in position.

The robotic arm assembly 700 also includes three active joints driven by motors. A yaw joint 710 allows arm section 730 to rotate around an axis 761, and a pitch joint 720 allows arm section 730 to rotate about an axis perpendicular to that of axis 761 and orthogonal to the plane of the drawing. The arm section 730 is configured so that sections 731 and 732 are always parallel to each other as the pitch joint 720 is rotated by its motor. As a consequence, the instrument 770 may be controllably moved by driving the yaw and pitch motors so as to pivot about the pivot point 762, which is generally located through manual positioning of the setup joints 704 and 705 so as to be at the point of incision into the patient. In addition, an insertion gear 745 may be coupled to a linear drive mechanism (not shown) to extend or retract the instrument 750 along its axis 763.

Although each of the yaw, pitch, and insertion joints or gears, 710, 720 and 745, is controlled by an individual joint or gear controller, the three controllers are controlled by a common master/slave control system so that the robotic arm assembly 700 (also referred to herein as a “slave manipulator”) may be controlled through user (e.g., surgeon) manipulation of its associated master manipulator.

While several embodiments have been shown in the drawings, it is not intended that the description be limited thereto, as it is intended that the description be as broad in scope as the art will allow and that the specification be read likewise. Therefore, the above description should not be construed as limiting, but merely as exemplifications of presently disclosed embodiments. Thus, the scope of the embodiments should be determined by the appended claims and their legal equivalents, rather than by the examples given.

Further, this description's terminology is not intended to limit the devices described herein. The term “force” is to be construed as encompassing both force and torque, unless otherwise indicated herein or clearly contradicted by context. The terms “tools” and “instruments” are used interchangeably herein to refer to the surgical instruments. As used in this specification and the appended claims, the singular forms “a,” “an,” and “the,” and any singular use of any word, include plural referents unless expressly and unequivocally limited to one referent. The terms “comprising,” “having,” “including,” and “containing” are to be construed as open-ended terms (i.e., meaning “including, but not limited to,”) unless otherwise noted. The terms “connected” and “coupled” are to be construed as partly or wholly contained within, attached to, or joined together, even if there is something intervening.

Spatially relative terms—such as “proximal” and “distal—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, the terms “proximal” and “distal” are relative terms, where the term “distal” refers to the portion of the object furthest from an operator of the instrument and closest to the surgical site, such as the opening of the tool cover or the end effector of the instrument. The term “proximal” indicates the relative proximity to the operator of the surgical instrument and refers to the portion of the object closest to the operator and furthest from the surgical site. In this application, an end effector refers to a tool installed at the distal end of an instrument, including but not limited to forceps or graspers, needle drivers, scalpels, scissors, spatulas, blades, and other tools, which may or may not use energy to cauterize tissue (i.e., a monopolar or bipolar tool).

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing from the disclosure. Accordingly, the present description is intended to embrace all such alternatives, modifications, and variances. As well, one skilled in the art will appreciate further features and advantages of the present disclosure based on the above-described embodiments. Accordingly, the present description is not to be limited by what has been particularly shown and described, except as indicated by the appended claims.

For example, in a first aspect, a first embodiment is a surgical instrument for applying surgical clips to tissue. The instrument comprises an elongate shaft having a longitudinal axis, an end effector coupled to the shaft and including first and second jaws movable between open and closed positions, a drive member configured to translate longitudinally through the shaft to deliver one or more surgical clips to the first and second jaws and an actuator extending through the shaft lateral of the drive member relative to the longitudinal axis and configured to move the jaws between the open and closed positions.

A second embodiment is the first embodiment, wherein longitudinal translation of the actuator causes the jaws to move between the open and closed positions.

A third embodiment is any combination of the first two embodiments, further comprising a second actuator lateral of the drive member relative to the longitudinal axis, wherein longitudinal translation of the first and second actuators causes the jaws to move between the open and closed positions.

A 4^{th} embodiment is any combination of the first 3 embodiments, wherein the actuator comprises an elongate rod having a distal end portion, and a slot pin coupled to the distal end portion, wherein the end effector comprises a slot and wherein the slot pin advances through the slot to move the jaws between the open and closed positions.

A 5^{th} embodiment is any combination of the first 4 embodiments, wherein the actuator comprises an elongate rod having a distal end portion, and first and second slot pins coupled to the distal end portion, wherein the end effector comprises a first slot coupled to the first jaw and a second slot coupled to the second jaw, wherein the first and second slot pins advance through the first and second slots, respectively, to move the jaws between the open and closed positions.

A 6^{th} embodiment is any combination of the first 5 embodiments, further comprising: a wrist assembly coupling the end effector with the shaft; and an internal tube extending through the wrist assembly, wherein the drive member is configured to translate through the internal channel.

A 7^{th} embodiment is any combination of the first 6 embodiments, wherein the actuator is disposed lateral of the internal tube.

An 8^{th} embodiment is any combination of the first 7 embodiments, wherein the wrist assembly pivotally couples the end effector with the shaft about an axis perpendicular to a shaft axis.

A 9^{th} embodiment is any combination of the first 8 embodiments, wherein the actuator comprises a grip cable having a flexible portion disposed within the wrist assembly and configured to bend as the wrist assembly articulates.

A 10^{th} embodiment is any combination of the first 9 embodiments, wherein the wrist assembly comprises distal and proximal links, wherein the grip cable comprises an outer sheath slidingly coupled to at least one of the distal and proximal links.

An 11^{th} embodiment is any combination of the first 10 embodiments, wherein the outer sheath of the grip cable is slidingly coupled to the proximal link and fixed to the distal link.

A 12^{th} embodiment is any combination of the first 11 embodiments, wherein the wrist assembly pivotally couples the end effector with the shaft about first and second axes, the first and second axes perpendicular to the shaft axis.

A 13^{th} embodiment is any combination of the first 12 embodiments, wherein the wrist assembly comprises a proximal link, a distal link and a middle link disposed between the proximal and distal links, wherein the grip cable comprises an outer sheath slidingly coupled to the proximal link and the middle link and fixed to the distal link.

A 14^{th} embodiment is any combination of the first 13 embodiments, wherein the grip cable further comprises proximal and distal portions on either side of the flexible portion, wherein the proximal and distal portions have a greater rigidity than the flexible portion.

A 15^{th} embodiment is any combination of the first 14 embodiments, wherein the drive member comprises a distal component for removably coupling to a surgical clip.

A 16^{th} embodiment is any combination of the first 15 embodiments, wherein the actuator comprises a proximal end portion configured for coupling to a robotic control system for translating the actuator to open and closed the jaws.

In another aspect, a first embodiment is a surgical instrument for applying surgical clips to tissue. the instrument comprise an elongate shaft having a longitudinal axis, a wrist assembly coupled to a distal end portion of the shaft, a flexible tube extending through the wrist and defining an internal channel, an end effector rotatably coupled to the wrist and including first and second jaws movable between open and closed positions and a drive member configured to translate longitudinally through the internal channel of the flexible tube to deliver one or more surgical clips to the first and second jaws.

A second embodiment is the first embodiment, wherein the flexible tube is configured to bend as the wrist assembly articulates relative to the end effector.

A third embodiment is any combination of the first two embodiments, wherein the wrist assembly is pivotally coupled the end effector about first and second axes, the first and second axes perpendicular to the shaft axis.

A 4^{th} embodiment is any combination of the first 3 embodiments, wherein the flexible tube is configured to bend in at least first and second locations on the flexible tube as the wrist assembly pivots about the first and second axes.

A 5^{th} embodiment is any combination of the first 4 embodiments, wherein the flexible tube comprises a coil and an elastic polymer sheath surrounding the coil.

A 6^{th} embodiment is any combination of the first 5 embodiments, wherein the flexible tube comprises a smooth internal surface.

A 7^{th} embodiment is any combination of the first 6 embodiments, wherein the flexible tube comprises a substantially D-shaped cross-section.

An 8^{th} embodiment is any combination of the first 7 embodiments, wherein the D-shaped cross section of the flexible tube comprises a semi-circular portion and a substantially linear portion, wherein the surgical instrument further comprises an actuator rod extending from the shaft to the end effector lateral of the substantially linear portion of the flexible tube, wherein the actuator rod is configured to move the jaws between the open and closed positions.

A 9^{th} embodiment is any combination of the first 8 embodiments, wherein the flexible tube comprises a cross-section having first and second substantially linear portions and first and second semi-circular portions extending between the linear portions.

A 10^{th} embodiment is any combination of the first 9 embodiments, further comprising first and second actuator rods extending from the shaft to the end effector and configured to move the jaws between the open and closed positions, wherein the first and second rods are lateral of the first and second substantially linear portions, respectively, of the flexible tube.

An 11^{th} embodiment is any combination of the first 10 embodiments, further comprising an actuator coupled to the wrist assembly for rotating the end effector relative to the shaft, wherein the actuator is configured for coupling to a robotic control system.

In another aspect, a first embodiment is a surgical instrument for applying surgical clips to tissue. The instrument comprises an elongate shaft having a longitudinal axis, an end effector including first and second jaws movable between open and closed positions, a wrist assembly comprising a first link coupled to the end effector rotatably coupling the end effector to the shaft about an axis perpendicular to the shaft and a drive member configured to translate longitudinal through the wrist assembly to deliver one or more surgical clips to the first and second jaws.

A second embodiment is the first embodiment, wherein the wrist assembly further comprises a second link rotatably coupled to the first link.

A third embodiment is any combination of the first two embodiments, wherein the wrist assembly further comprises a middle link between the first and second links, wherein the middle link is rotatably coupled to the first and second links about a second axis perpendicular to the first axis and perpendicular to the shaft.

A 4^{th} embodiment is any combination of the first 3 embodiments, wherein the second link is rigidly attached to the end effector.

A 5^{th} embodiment is any combination of the first 4 embodiments, further comprising an actuator extending through the wrist assembly lateral of the drive member and being configured to move the jaws between the open and closed positions.

A 6^{th} embodiment is any combination of the first 5 embodiments, wherein the actuator comprises a rod having a flexible portion extending through the wrist assembly.

A 7^{th} embodiment is any combination of the first 6 embodiments, wherein the flexible portion is configured to expand and contract along a longitudinal axis of the flexible portion as the distal link articulates relative to the proximal link.

An 8^{th} embodiment is any combination of the first 7 embodiments, wherein the flexible portion comprises an outer sheath secured to the distal link and slidingly coupled to the proximal link.

A 9^{th} embodiment is any combination of the first 8 embodiments, wherein the drive member comprises a distal component for removably coupling to a surgical clip.

A 10^{th} embodiment is any combination of the first 9 embodiments, wherein the first and second jaws include first and second guide tracks, respectively, for receiving protrusions on the surgical clip to guide the surgical clip to the jaws.

An 11^{th} embodiment is any combination of the first 10 embodiments, wherein the surgical clip includes first and second arms, and wherein at least one of the first and second jaws comprises an engagement element for securing at least one of the first and second arms of the clip to said at least one of the first and second jaws.

1. A surgical instrument for applying surgical clips to tissue, the instrument comprising:
an elongate shaft having a longitudinal axis;
an end effector coupled to the shaft and including a first jaw and a second jaw;
a drive member configured to translate longitudinally through the shaft to deliver a surgical clip to the first and second jaws, the drive member comprising an engagement element for removably coupling to a first arm and a second arm of the surgical clip; and
an actuator extending through the shaft, the actuator disposed lateral of the drive member relative to the longitudinal axis and configured to move at least one of the jaws between the open and closed positions.

2. The instrument of claim 1, wherein longitudinal translation of the actuator causes at least one of the jaws to move between the open and closed positions.

3. The instrument of claim 1, further comprising a second actuator disposed lateral of the drive member relative to the longitudinal axis, wherein longitudinal translation of the first and second actuators causes the jaws to move between the open and closed positions.

4. The instrument of claim 1, wherein the actuator comprises an elongate rod having a distal end portion, and a slot pin coupled to the distal end portion, wherein the end effector comprises a slot and wherein the slot pin advances through the slot to move the jaws between the open and closed positions.

5. The instrument of claim 1, wherein the actuator comprises an elongate rod having a distal end portion, and first and second slot pins coupled to the distal end portion, wherein the end effector comprises a first slot coupled to the first jaw and a second slot coupled to the second jaw, wherein the first and second slot pins advance through the first and second slots, respectively, to move the jaws between the open and closed positions.

6. The instrument of claim 1, further comprising:
a wrist assembly coupling the end effector with the shaft; and
an internal tube extending through the wrist assembly, wherein the drive member is configured to translate through the internal channel.

7. (canceled)

8. The instrument of claim 6, wherein the wrist assembly pivotally couples the end effector with the shaft about an axis perpendicular to a shaft axis.

9. The instrument of claim 8, wherein the actuator comprises a grip cable having a flexible portion disposed within the wrist assembly and configured to bend as the wrist assembly articulates.

10. The instrument of claim 9, wherein the wrist assembly comprises a distal link and a proximal link, wherein the grip cable comprises an outer sheath slidingly coupled to at least one of the distal and proximal links.

11. The instrument of claim 10, wherein the outer sheath of the grip cable is slidingly coupled to the proximal link and fixed to the distal link.

12. The instrument of claim 6, wherein the wrist assembly pivotally couples the end effector with the shaft about first and second axes, the first and second axes perpendicular to the shaft axis.

13. The instrument of claim 12, wherein the wrist assembly comprises a proximal link, a distal link and a middle link disposed between the proximal and distal links, wherein the grip cable comprises an outer sheath slidingly coupled to the proximal link and the middle link and fixed to the distal link.

14. (canceled)

15. The instrument of claim 1, wherein the drive member comprises a distal component for removably coupling to the a surgical clip.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. A surgical instrument for applying surgical clips to tissue, the instrument comprising:
an elongate shaft having a longitudinal axis;
an end effector including a first jaw and a second jaw;
a wrist assembly comprising a first link coupled to the end effector rotatably coupling the end effector to the shaft about an a first axis perpendicular to the longitudinal axis;
a drive member configured to translate longitudinal through the wrist assembly to deliver surgical clip to the first and second jaws, the surgical clip comprising first and second protrusions and wherein each of the first and second jaws include respective first and second guide tracks for receiving the first and second protrusions on the surgical clip to guide the surgical clip into the first and second jaws.

29. The surgical instrument of claim 28, wherein the wrist assembly further comprises a second link rotatably coupled to the first link.

30. The surgical instrument of claim 29, wherein the wrist assembly further comprises a middle link between the first and second links, wherein the middle link is rotatably coupled to the first and second links about a second axis perpendicular to the first axis and perpendicular to the shaft.

31. (canceled)

32. The surgical instrument of claim 29, further comprising an actuator extending through the wrist assembly lateral of the drive member and being configured to move the jaws between the open and closed positions, wherein the actuator comprises a rod having a flexible portion extending through the wrist assembly.

33. (canceled)

34. The surgical instrument of claim 32, wherein the flexible portion is configured to expand and contract along a longitudinal axis of the flexible portion as the first link articulates relative to the second link, wherein the flexible portion comprises an outer sheath secured to the first link and slidingly coupled to the second link.

35. (canceled)

36. The surgical instrument of claim 28, wherein the drive member comprises a distal component for removably coupling to a surgical clip.

37. (canceled)

38. The surgical instrument of claim 28, wherein the surgical clip includes first and second arms, and wherein at least one of the first and second jaws comprises an engagement element for securing at least one of the first and second arms of the clip to said at least one of the first and second jaws.
